(19) United States
(12) Patent Application Publication
(10) Pub. No.: US 2014/0057323 A1
(43) Pub. Date: Feb. 27, 2014

(54) ENHANCED CELLODEXTRIN METABOLISM

(71) Applicants: James H. Doudna Cate, Berkeley, CA (US); Yong-Su Jin, Champaign, IL (US); Jonathan M. Galazka, Berkeley, CA (US); Suk-Jin Ha, Savoy, IL (US)

(72) Inventors: James H. Doudna Cate, Berkeley, CA (US); Yong-Su Jin, Champaign, IL (US); Jonathan M. Galazka, Berkeley, CA (US); Suk-Jin Ha, Savoy, IL (US)

(73) Assignees: The Board of Trustees of the University of Illinois, Urbana, IL (US); The Regents of the University of California, Oakland, CA (US)

(21) Appl. No.: 13/982,248

(22) PCT Filed: Feb. 7, 2012
(86) PCT No.: PCT/US2012/024186

§ 371 Date: Oct. 23, 2013

Related U.S. Application Data
(60) Provisional application No. 61/440,305, filed on Feb. 7, 2011.
(60) Provisional application No. 61/566,548, filed on Dec. 2, 2011.

(51) Int. Cl. C12P 19/18; C12P 19/02

(52) U.S. Cl. CPC C12P 19/18; C12P 19/02
USPC 435/97; 435/254.21

(57) ABSTRACT

The present disclosure relates to host cells containing two or more of a recombinant cellodextrin transporter, a recombinant cellodextrin phosphorylase, a recombinant β-glucosidase, a recombinant phosphoglucomutase, or a recombinant hexokinase; and to methods of using such cells for degrading cellodextrin, for producing hydrocarbons or hydrocarbon derivatives from cellodextrin, and for reducing ATP consumption during glucose utilization.

### CROSS-REFERENCE TO RELATED APPLICATIONS

This application claims the benefit of U.S. Provisional Application No. 61/440,305, filed Feb. 7, 2011, and U.S. Provisional Application No. 61/566,548, filed Dec. 2, 2011, both of which are hereby incorporated by reference in their entirety.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 677792001340SeqList.txt, date recorded: Feb. 6, 2012, size: 1209 KB).

### FIELD

The present disclosure relates to methods and compositions for degrading cellodextrin and for producing hydrocarbons and hydrocarbon derivatives.

### BACKGROUND

Biofuels are under intensive investigation due to increasing concerns about energy security, sustainability, and global climate change (Lynd et al., Science, 1991). Bioconversion of plant-derived lignocellulosic materials into biofuels has been regarded as an attractive alternative to chemical production of fossil fuels (Lynd et al., Nat Biotech, 2008; Hahn-Hagerdal et al., Biotechnol Biofuels, 2006). The engineering of microorganisms to perform the conversion of lignocellulosic biomass to ethanol efficiently remains a major goal of the biofuels field. Much research has been focused on genetically manipulating microorganisms that naturally ferment simple sugars to alcohol to express cellulases and other enzymes that would allow them to degrade lignocellulosic biomass polymers and generate ethanol within one cell, a process known as consolidated bioprocessing (CBP).

Saccharomyces cerevisiae, also known as baker's yeast, has been used for bioconversion of simple hexose sugars into ethanol for thousands of years. It is also the most widely used microorganism for large scale industrial fermentation of D-glucose into ethanol. S. cerevisiae is a very suitable candidate for bioconversion of lignocellulosic biomass into biofuels (van Maris et al., Antonie Van Leeuwenhoek, 2006). It has a well-studied genetic and physiological background, ample genetic tools, and high tolerance to high ethanol concentration and inhibitors present in lignocellulosic hydrolysates (Jeffries, Curr Opin Biotechnol, 2006). The low fermentation pH of S. cerevisiae can also prevent bacterial contamination during fermentation.

S. cerevisiae, however, does not naturally degrade and ferment the more complex biomass polymers, such as cellulose, that are present in plant cell walls. Enzymes useful for the degradation of biomass polymers have been sought after in those organisms that naturally degrade biomass, such as Neurospora crassa and Trichoderma reesei. A recent study of plant wall degradation in N. crassa showed that in addition to the expression of various cellulases, N. crassa expresses cellodextrin transporters and an intracellular β-glucosidase in response to cellulose (Tian et al., PNAS USA 106, 22157, 2009; Galazka et al., Science 330, 84, 2010). Cellodextrins are β (1→4) linked oligosaccharides of glucose and are the product of cellulose depolymerization by fungal cellulases (Zhang and Lynd, Biotechnol Bioeng 88, 797, 2004). β-glucosidase hydrolyzes cellodextrins to glucose.

S. cerevisiae engineered to express a cellodextrin transporter and an intracellular β-glucosidase are able to grow with cellodextrins as the sole carbon source and ferment cellobiose to ethanol efficiently (Galazka et al., Science 330, 84, 2010). However using a β-glucosidase to hydrolyze cellodextrins to glucose requires that all produced glucose be phosphorylated to glucose-6-phosphate in a reaction that consumes 1 ATP per glucose before further processing can occur. This is problematic when ATP is in short supply.

In addition, transport of cellodextrins followed by intracellular hydrolysis facilitates the co-fermentation of cellulose-derived glucose and hemicellulose-derived xylose, which S. cerevisiae is normally unable to do. However, these engineered strains may not ferment glucose with optimal metabolism because their endogenous system for detecting and responding to the presence of glucose is dependent on the extracellular level of glucose. In the engineered strains, the extracellular level of glucose is no longer tied to the level of glucose available to the cell since glucose is generated intracellularly by transporting cellodextrins into the cell and intracellularly degrading the cellodextrins to glucose.

Accordingly, a need exists for improved engineered yeast strains that can perform consolidated bioprocessing of biomass polymers to biofuels and other useful chemicals, and that consume less ATP when phosphorylating the glucose produced from the cleavage of cellodextrins.

### BRIEF SUMMARY

In order to meet the above needs, the present disclosure provides host cells containing two or more of a recombinant cellodextrin transporter, a recombinant cellodextrin phosphorylase, a recombinant β-glucosidase, a recombinant phosphoglucomutase, or a recombinant hexokinase; and methods of using such cells for degrading cellodextrin, for producing hydrocarbons or hydrocarbon derivatives from cellodextrin, and for reducing ATP consumption during cellodextrin utilization. Moreover, the present disclosure is based at least in part on the novel discovery that yeast strains engineered to express an intracellular cellodextrin phosphorylase rather than a β-glucosidase consumed less ATP in the utilization of cellodextrins and produced nearly equivalent amounts of ethanol as compared to a yeast strain that expresses a β-glucosidase. Advantageously, cellodextrin phosphorylases utilize inorganic phosphate to cleave the β-glucosidic linkage between glucose moieties in cellodextrins. The phosphorolysis reaction saves 1 ATP equivalent per cleavage reaction and results in the release of glucose-1-phosphate (FIG. 1). The resulting glucose-1-phosphate can then be converted to glucose-6-phosphate by phosphoglucomutases (FIG. 1). Moreover, the yeast can directly utilize the resulting glucose-6-phosphate for growth and fermentation.

Accordingly, certain aspects of the present disclosure relate to a method for degrading cellodextrin, by: a) providing a host cell containing a recombinant cellodextrin transporter and a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity; and b) culturing the host cell in a medium containing cellodextrin or a source of cellodextrin, whereby cellodextrin is transported into the cell and degraded by the recombinant polypeptide. Other aspects of the present disclosure relate to a method for producing hydrocarbons or hydrocarbon derivatives from cellodextrin, by: a) providing a host cell containing a recombinant cellodextrin transporter and a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity; and b) culturing the host cell in a medium containing cellodextrin or a source of cellodextrin, whereby cellodextrin is transported into the cell and degraded by the recombinant polypeptide and whereby the host cell produces hydrocarbons or hydrocarbon derivatives from the cellodextrin. Other aspects of the present disclosure relate to a method for reducing ATP consumption during glucose utilization, by: a) providing a host cell containing a recombinant cellodextrin transporter and a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity; and b) culturing the host cell in a medium containing cellodextrin or a source of cellodextrin, whereby cellodextrin is transported into the cell and degraded by the recombinant polypeptide to glucose-1-phosphate, where the production of glucose-1-phosphate from cellodextrin reduces ATP consumption as compared to a corresponding cell lacking the recombinant polypeptide. In certain embodiments that may be combined with any of the preceding embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments that may be combined with any of the preceding embodiments, the recombinant polypeptide has cellobiose phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the recombinant polypeptide contains one or more mutations. In certain embodiments, the one or more mutations are amino acid substitutions. In certain embodiments that may be combined with any of the preceding embodiments, the recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof. In certain embodiments that may be combined with any of the preceding embodiments, the host cell further contains a recombinant phosphoglucomutase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the host cell further contains a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1.

Some aspects of the present disclosure relate to a method for degrading cellodextrin, by: a) providing a host cell containing a recombinant cellodextrin transporter and a recombinant phosphoglucomutase; and b) culturing the host cell in a medium containing cellodextrin or a source of cellodextrin, whereby cellodextrin is transported into the cell and degraded. Other aspects of the present disclosure relate to a method for producing hydrocarbons or hydrocarbon derivatives from cellodextrin, by: a) providing a host cell containing a recombinant cellodextrin transporter and a recombinant phosphoglucomutase; and b) culturing the host cell in a medium containing cellodextrin or a source of cellodextrin, whereby cellodextrin is transported into the cell and whereby the host cell produces hydrocarbons or hydrocarbon derivatives from the transported cellodextrin. In certain embodiments that may be combined with any of the preceding embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the host cell further contains a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1.

Some aspects of the present disclosure relate to a method for degrading cellodextrin, by: a) providing a host cell containing a recombinant cellodextrin transporter and a recombinant hexokinase; and b) culturing the host cell in a medium containing cellodextrin or a source of cellodextrin, whereby cellodextrin is transported into the cell and degraded. Other aspects of the present disclosure relate to a method for producing hydrocarbons or hydrocarbon derivatives from cellodextrin, by: a) providing a host cell containing a recombinant cellodextrin transporter and a recombinant hexokinase; and b) culturing the host cell in a medium containing cellodextrin or a source of cellodextrin, whereby cellodextrin is transported into the cell and whereby the host cell produces hydrocarbons or hydrocarbon derivatives from the transported cellodextrin. In certain embodiments that may be combined with any of the preceding embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1. In certain embodiments that may be combined with any of the preceding embodiments, the host cell further contains a recombinant phosphoglucomutase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19).

In certain embodiments that may be combined with any of the preceding embodiments, the host cell further contains a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments, the recombinant polypeptide has cellobiose phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the recombinant polypeptide contains one or more mutations. In certain embodiments, the one or more mutations are amino acid substitutions. In certain embodiments that may be combined with any of the preceding embodiments, the recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof. In certain embodiments that may be combined with any of the preceding embodiments, the recombinant polypeptide reduces ATP consumption as compared to a corresponding cell lacking the recombinant polypeptide. In certain embodiments that may be combined with any of the preceding embodiments, the host cell further contains a second recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the second recombinant polypeptide has β-glucosidase activity. In certain embodiments, the second recombinant polypeptide contains two or more sequences selected from F-x-[FYWM]-[GSTA]-x-[GSTA]-x-[GSTA](2)-[FYNH]-[NQ]-x-E-x-[GSTA] (SEQ ID NO: 16), [LIVMFSTC]-[LIVFYS]-[LIV]-[LIVMST]-E-N-G-[LIVMFAR]-[CSAGN] (SEQ ID NO: 17), and [LIVM](2)-[KR]-x-[EQKRD]-x(4)-G-[LIVMFTC]-[LIVT]-[LIVMF]-[ST]-D-x(2)-[SGADNIT] (SEQ ID NO: 18). In certain embodiments that may be combined with any of the preceding embodiments, the second recombinant polypeptide contains an amino acid sequence that is least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identical to the amino acid sequence of NCU00130.

Some aspects of the present disclosure relate to a method for degrading cellodextrin, by: a) providing a host cell containing a recombinant cellodextrin transporter and a recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity; and b) culturing the host cell in a medium containing cellodextrin or a source of cellodextrin, whereby cellodextrin is transported into the cell and degraded by the recombinant polypeptide. Other aspects of the present disclosure relate to a method for producing hydrocarbons or hydrocarbon derivatives from cellodextrin, by: a) providing a host cell containing a recombinant cellodextrin transporter and a recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity; and b) culturing the host cell in a medium containing cellodextrin or a source of cellodextrin, whereby cellodextrin is transported into the cell and degraded by the recombinant polypeptide and whereby the host cell produces hydrocarbons or hydrocarbon derivatives from the cellodextrin. In certain embodiments that may be combined with any of the preceding embodiments, the recombinant polypeptide contains two or more sequences selected from F-x-[FYWM]-[GSTA]-x-[GSTA]-x-[GSTA](2)-[FYNH]-[NQ]-x-E-x-[GSTA] (SEQ ID NO: 16), [LIVMFSTC]-[LIVFYS]-[LIV]-[LIVMST]-E-N-G-[LIVMFAR]-[CSAGN] (SEQ ID NO: 17), and [LIVM](2)-[KR]-x-[EQKRD]-x(4)-G-[LIVMFTC]-[LIVT]-[LIVMF]-[ST]-D-x(2)-[SGADNIT] (SEQ ID NO: 18). In certain embodiments that may be combined with any of the preceding embodiments, the recombinant polypeptide contains an amino acid sequence that is least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identical to the amino acid sequence of NCU00130. In certain embodiments that may be combined with any of the preceding embodiments, the host cell further contains a recombinant phosphoglucomutase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the host cell further contains a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1. In certain embodiments that may be combined with any of the preceding embodiments, the host cell further contains a second recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the second recombinant polypeptide has cellodextrin phosphorylase activity. In certain embodiments, the second recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments, the second recombinant polypeptide has cellobiose phosphorylase activity. In certain embodiments, the second recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the second recombinant polypeptide contains one or more mutations. In certain embodiments, the one or more mutations are amino acid substitutions. In certain embodiments that may be combined with any of the preceding embodiments, the second recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof.

In certain embodiments that may be combined with any of the preceding embodiments, the recombinant cellodextrin transporter contains a polypeptide selected from a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 1 contains SEQ ID NO: 1; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 2 contains SEQ ID NO: 2; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and a loop connecting transmembrane α-helix 2 and transmembrane α-helix 3 contains SEQ ID NO: 3; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 5 contains SEQ ID NO: 4; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 6 contains SEQ ID NO: 5; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and sequence between transmembrane α-helix 6 and transmembrane α-helix 7 contains SEQ ID NO: 6; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 7 contains SEQ ID NO: 7; and a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 10 and transmembrane α-helix 11 and the sequence between them contain SEQ ID NO: 8. In certain embodiments, the recombinant cellodextrin transporter is a cellobiose transporter. In certain embodiments, the cellobiose transporter has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to SEQ ID NO: 9 (CDT-1) or SEQ ID NO: 10 (CDT-2). In certain embodiments that may be combined with any of the preceding embodiments, the recombinant cellodextrin transporter contains one or more mutations. In certain embodiments, the one or more mutations are amino acid substitutions. In certain embodiments that may be combined with any of the preceding embodiments, the recombinant cellodextrin transporter contains an amino acid substitution at one or more positions corresponding to positions of the amino acid sequence of SEQ ID NO: 9 (CDT-1), where the one or more positions are at positions selected from a position corresponding to amino acid 91 of SEQ ID NO: 9, a position corresponding to amino acid 104 of SEQ ID NO: 9, a position corresponding to amino acid 170 of SEQ ID NO: 9, a position corresponding to amino acid 174 of SEQ ID NO: 9, a position corresponding to amino acid 194 of SEQ ID NO: 9, a position corresponding to amino acid 213 of SEQ ID NO: 9, a position corresponding to amino acid 335 of SEQ ID NO: 9, and combinations thereof. In certain embodiments that may be combined with any of the preceding embodiments, the recombinant cellodextrin transporter contains an amino acid substitution at one or more positions corresponding to positions of the amino acid sequence of SEQ ID NO: 9 (CDT-1), where the one or more amino acid substitutions are at selected from a glycine (G) to alanine (A) substitution at a position corresponding to amino acid 91 of SEQ ID NO: 9, a glutamine (Q) to alanine (A) substitution at a position corresponding to amino acid 104 of SEQ ID NO: 9, a phenylalanie (F) to alanine (A) substitution at a position corresponding to amino acid 170 of SEQ ID NO: 9, an arginine (R) to alanine (A) substitution at a position corresponding to amino acid 174 of SEQ ID NO: 9, a glutamate (E) to alanine (A) substitution at a position corresponding to amino acid 194 of SEQ ID NO: 9, a phenylalanie (F) to lysine (L) substitution at a position corresponding to amino acid 213 of SEQ ID NO: 9, a phenylalanie (F) to alanine (A) substitution at a position corresponding to amino acid 335 of SEQ ID NO: 9, and combinations thereof.

Some aspects of the present disclosure relate to a method for degrading cellodextrin, by: a) providing a host cell containing a recombinant phosphoglucomutase, and a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity; and b) culturing the host cell in a medium containing cellodextrin or a source of cellodextrin, whereby cellodextrin is degraded by the recombinant polypeptide. Other aspects of the present disclosure relate to a method for producing hydrocarbons or hydrocarbon derivatives from cellodextrin, by: a) providing a host cell containing a recombinant phosphoglucomutase, and a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity; and b) culturing the host cell in a medium containing cellodextrin or a source of cellodextrin, whereby cellodextrin is degraded by the recombinant polypeptide and whereby the host cell produces hydrocarbons or hydrocarbon derivatives from the cellodextrin. Other aspects of the present disclosure relate to a method for reducing ATP consumption during glucose utilization, by: a) providing a host cell containing a recombinant phosphoglucomutase, and a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity; and b) culturing the host cell in a medium containing cellodextrin or a source of cellodextrin, whereby cellodextrin is degraded by the recombinant polypeptide to glucose-1-phosphate, where the production of glucose-1-phosphate from cellodextrin reduces ATP consumption as compared to a corresponding cell lacking the recombinant polypeptide. In certain embodiments that may be combined with any of the preceding embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the host cell further contains a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1.

Some aspects of the present disclosure relate to a method for degrading cellodextrin, by: a) providing a host cell containing a recombinant hexokinase, and a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity; and b) culturing the host cell in a medium containing cellodextrin or a source of cellodextrin, whereby cellodextrin is degraded by the recombinant polypeptide. Other aspects of the present disclosure relate to a method for producing hydrocarbons or hydrocarbon derivatives from cellodextrin, by: a) providing a host cell containing a recombinant hexokinase, and a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity; and b) culturing the host cell in a medium containing cellodextrin or a source of cellodextrin, whereby cellodextrin is degraded by the recombinant polypeptide and whereby the host cell produces hydrocarbons or hydrocarbon derivatives from the cellodextrin. Other aspects of the present disclosure relate to a method for reducing ATP consumption during glucose utilization, by: a) providing a host cell containing a recombinant hexokinase, and a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity; and b) culturing the host cell in a medium containing cellodextrin or a source of cellodextrin, whereby cellodextrin is degraded by the recombinant polypeptide to glucose-1-phosphate, where the production of glucose-1-phosphate from cellodextrin reduces ATP consumption as compared to a corresponding cell lacking the recombinant polypeptide. In certain embodiments that may be combined with any of the preceding embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1. In certain embodiments that may be combined with any of the preceding embodiments, the host cell further contains a recombinant phosphoglucomutase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments that may be combined with any of the preceding embodiments, the recombinant polypeptide has cellobiose phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the recombinant polypeptide contains one or more mutations. In certain embodiments, the one or more mutations are amino acid substitutions. In certain embodiments that may be combined with any of the preceding embodiments, the recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof.

Some aspects of the present disclosure relate to a method for degrading cellodextrin, by: a) providing a host cell containing a recombinant phosphoglucomutase and a recombinant hexokinase; and b) culturing the host cell in a medium containing cellodextrin or a source of cellodextrin, whereby cellodextrin is degraded. Other aspects of the present disclosure relate to a method for producing hydrocarbons or hydrocarbon derivatives from cellodextrin, by: a) providing a host cell containing a recombinant phosphoglucomutase and a recombinant hexokinase; and b) culturing the host cell in a medium containing cellodextrin or a source of cellodextrin, whereby cellodextrin is degraded and whereby the host cell produces hydrocarbons or hydrocarbon derivatives from the cellodextrin. In certain embodiments that may be combined with any of the preceding embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1. In certain embodiments that may be combined with any of the preceding embodiments, the host cell further contains a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments, the recombinant polypeptide has cellobiose phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the recombinant polypeptide contains one or more mutations. In certain embodiments, the one or more mutations are amino acid substitutions. In certain embodiments that may be combined with any of the preceding embodiments, the recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof. In certain embodiments that may be combined with any of the preceding embodiments, the recombinant polypeptide reduces ATP consumption as compared to a corresponding cell lacking the recombinant polypeptide.

In certain embodiments that may be combined with any of the preceding embodiments, the host cell further contains a second recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the second recombinant polypeptide has β-glucosidase activity. In certain embodiments, the second recombinant polypeptide contains two or more sequences selected from F-x-[FYWM]-[GSTA]-x-[GSTA]-x-[GSTA](2)-[FYNH]-[NQ]-x-E-x-[GSTA] (SEQ ID NO: 16), [LIVMFSTC]-[LIVFYS]-[LIV]-[LIVMST]-E-N-G-[LIVMFAR]-[CSAGN] (SEQ ID NO: 17), and [LIVM](2)-[KR]-x-[EQKRD]-x(4)-G-[LIVMFTC]-[LIVT]-[LIVMF]-[ST]-D-x(2)-[SGADNIT] (SEQ ID NO: 18). In certain embodiments that may be combined with any of the preceding embodiments, the second recombinant polypeptide contains an amino acid sequence that is least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identical to the amino acid sequence of NCU00130.

Some aspects of the present disclosure relate to a method for degrading cellodextrin, by: a) providing a host cell containing: a first recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the first recombinant polypeptide has cellodextrin phosphorylase activity, and a second recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the second recombinant polypeptide has β-glucosidase activity; and b) culturing the host cell in a medium containing cellodextrin or a source of cellodextrin, whereby cellodextrin is degraded by the recombinant polypeptides. Other aspects of the present disclosure relate to a method for producing hydrocarbons or hydrocarbon derivatives from cellodextrin, by: a) providing a host cell containing: a first recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS](SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the first recombinant polypeptide has cellodextrin phosphorylase activity, and a second recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the second recombinant polypeptide has β-glucosidase activity; and b) culturing the host cell in a medium containing cellodextrin or a source of cellodextrin, whereby cellodextrin is degraded by the recombinant polypeptides and whereby the host cell produces hydrocarbons or hydrocarbon derivatives from the cellodextrin. In certain embodiments that may be combined with any of the preceding embodiments, the first recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments that may be combined with any of the preceding embodiments, the first recombinant polypeptide has cellobiose phosphorylase activity. In certain embodiments, the first recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the first recombinant polypeptide contains one or more mutations. In certain embodiments, the one or more mutations are amino acid substitutions. In certain embodiments that may be combined with any of the preceding embodiments, the first recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof. In certain embodiments that may be combined with any of the preceding embodiments, the host cell further contains a recombinant phosphoglucomutase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the host cell further contains a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1.

Some aspects of the present disclosure relate to a method for degrading cellodextrin, by: a) providing a host cell containing a recombinant phosphoglucomutase, and a recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity; and b) culturing the host cell in a medium containing cellodextrin or a source of cellodextrin, whereby cellodextrin is degraded by the recombinant polypeptide. Other aspects of the present disclosure relate to a method for producing hydrocarbons or hydrocarbon derivatives from cellodextrin, by: a) providing a host cell containing a recombinant phosphoglucomutase, and a recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity; and b) culturing the host cell in a medium containing cellodextrin or a source of cellodextrin, whereby cellodextrin is degraded by the recombinant polypeptide and whereby the host cell produces hydrocarbons or hydrocarbon derivatives from the cellodextrin. In certain embodiments that may be combined with any of the preceding embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the host cell further contains a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1.

Some aspects of the present disclosure relate to a method for degrading cellodextrin, by: a) providing a host cell containing a recombinant hexokinase, and a recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity; and b) culturing the host cell in a medium containing cellodextrin or a source of cellodextrin, whereby cellodextrin is degraded by the recombinant polypeptide. Other aspects of the present disclosure relate to a method for producing hydrocarbons or hydrocarbon derivatives from cellodextrin, by: a) providing a host cell containing a recombinant hexokinase, and a recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity; and b) culturing the host cell in a medium containing cellodextrin or a source of cellodextrin, whereby cellodextrin is degraded by the recombinant polypeptide and whereby the host cell produces hydrocarbons or hydrocarbon derivatives from the cellodextrin. In certain embodiments that may be combined with any of the preceding embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1. In certain embodiments that may be combined with any of the preceding embodiments, the host cell further contains a recombinant phosphoglucomutase. In certain embodiments the recombinant phosphoglucomutase contains a conserved motif having the amino acid sequence of [GSA]-[LIVMF]-x-[LIVM]-[ST]-[PGA]-S-H-[NIC]-P (SEQ ID NO: 19).

In certain embodiments that may be combined with any of the preceding embodiments, the host cell further contains a second recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the second recombinant polypeptide has cellodextrin phosphorylase activity. In certain embodiments, the second recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments, the second recombinant polypeptide has cellobiose phosphorylase activity. In certain embodiments, the second recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the second recombinant polypeptide contains one or more mutations. In certain embodiments, the one or more mutations are amino acid substitutions. In certain embodiments that may be combined with any of the preceding embodiments, the second recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof.

In certain embodiments that may be combined with any of the preceding embodiments, the host cell further contains a recombinant cellodextrin transporter containing a polypeptide selected from a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 1 contains SEQ ID NO: 1; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 2 contains SEQ ID NO: 2; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and a loop connecting transmembrane α-helix 2 and transmembrane α-helix 3 contains SEQ ID NO: 3; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 5 contains SEQ ID NO: 4; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 6 contains SEQ ID NO: 5; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and sequence between transmembrane α-helix 6 and transmembrane α-helix 7 contains SEQ ID NO: 6; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 7 contains SEQ ID NO: 7; and a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 10 and transmembrane α-helix 11 and the sequence between them contain SEQ ID NO: 8. In certain embodiments, the recombinant cellodextrin transporter is a cellobiose transporter. In certain embodiments, the cellobiose transporter has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to SEQ ID NO: 9 (CDT-1) or SEQ ID NO: 10 (CDT-2). In certain embodiments that may be combined with any of the preceding embodiments, the recombinant cellodextrin transporter contains one or more mutations. In certain embodiments, the one or more mutations are amino acid substitutions. In certain embodiments that may be combined with any of the preceding embodiments, the recombinant cellodextrin transporter contains an amino acid substitution at one or more positions corresponding to positions of the amino acid sequence of SEQ ID NO: 9 (CDT-1), where the one or more positions are at positions selected from a position corresponding to amino acid 91 of SEQ ID NO: 9, a position corresponding to amino acid 104 of SEQ ID NO: 9, a position corresponding to amino acid 170 of SEQ ID NO: 9, a position corresponding to amino acid 174 of SEQ ID NO: 9, a position corresponding to amino acid 194 of SEQ ID NO: 9, a position corresponding to amino acid 213 of SEQ ID NO: 9, a position corresponding to amino acid 335 of SEQ ID NO: 9, and combinations thereof. In certain embodiments that may be combined with any of the preceding embodiments, the recombinant cellodextrin transporter contains an amino acid substitution at one or more positions corresponding to positions of the amino acid sequence of SEQ ID NO: 9 (CDT-1), where the one or more amino acid substitutions are at selected from a glycine (G) to alanine (A) substitution at a position corresponding to amino acid 91 of SEQ ID NO: 9, a glutamine (Q) to alanine (A) substitution at a position corresponding to amino acid 104 of SEQ ID NO: 9, a phenylalanie (F) to alanine (A) substitution at a position corresponding to amino acid 170 of SEQ ID NO: 9, an arginine (R) to alanine (A) substitution at a position corresponding to amino acid 174 of SEQ ID NO: 9, a glutamate (E) to alanine (A) substitution at a position corresponding to amino acid 194 of SEQ ID NO: 9, a phenylalanie (F) to lysine (L) substitution at a position corresponding to amino acid 213 of SEQ ID NO: 9, a phenylalanie (F) to alanine (A) substitution at a position corresponding to amino acid 335 of SEQ ID NO: 9, and combinations thereof. In certain embodiments that may be combined with any of the preceding embodiments, the recombinant polypeptide having β-glucosidase activity contains two or more sequences selected from F-x-[FYWM]-[GSTA]-x-1-x-[GSTA]-x-[GSTA](2)-[FYNH]-[NQ]-x-E-x-[GSTA] (SEQ ID NO: 16), [LIVMFSTC]-[LIVFYS]-[LIV]-[LIVMST]-E-N-G-[LIVMFAR]-[CSAGN] (SEQ ID NO: 17), and [LIVM](2)-[KR]-x-[EQKRD]-x(4)-G-[LIVMFTC]-[LIVT]-[LIVMF]-[ST]-D-x(2)-[SGADNIT] (SEQ ID NO: 18). In certain embodiments that may be combined with any of the preceding embodiments, the recombinant polypeptide β-glucosidase activity contains an amino acid sequence that is least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identical to the amino acid sequence of NCU00130.

In certain embodiments that may be combined with any of the preceding embodiments, the host cell further contains one or more glucose response genes, where the activity level of a protein encoded by at least one glucose response gene is altered compared to the wild-type activity level of the protein. In certain embodiments, the one or more glucose response genes are selected from Snf3, Rgt1, Rgt2, Yck1/2, Std1, Mthy1, Snf1/4, Grr1, Gpr1, Gpa2, Ras2, Stb3, Hxk2, Pfk27, Pfk26, Sch9, Yak1, Mig1, Rim15, Kcs1, and Tps1. In certain embodiments that may be combined with any of the preceding embodiments, the activity level of one or more proteins encoded by the one or more glucose response genes is increased compared to its wild-type activity level. In certain embodiments that may be combined with any of the preceding embodiments, the activity level of one or more proteins encoded by the one or more glucose response genes is decreased compared to its wild-type activity level. In certain embodiments that may be combined with any of the preceding embodiments, the source of cellodextrin contains cellulose. In certain embodiments that may be combined with any of the preceding embodiments, the cellodextrin is selected from one or more of the group consisting of cellobiose, cellotriose, cellotetraose, cellopentose, and cellohexose. In certain embodiments that may be combined with any of the preceding embodiments, the hydrocarbons or hydrocarbon derivatives can be used as fuel. In certain embodiments that may be combined with any of the preceding embodiments, the hydrocarbons or hydrocarbon derivatives contain ethanol. In certain embodiments, the ethanol is produced at a rate that ranges from at least about 0.10 to at least 20 g/L-h. In certain embodiments that may be combined with any of the preceding embodiments, the hydrocarbons or hydrocarbon derivatives contain butanol. In certain embodiments that may be combined with any of the preceding embodiments, the host cell is a fungal cell. In certain embodiments that may be combined with any of the preceding embodiments, the host cell is a yeast cell. In certain embodiments, the yeast cell is S. cerevisiae.

Other aspects of the present disclosure relate to a host cell containing a recombinant cellodextrin transporter, and a recombinant polypeptide containing G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 14) or Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 15), where the recombinant polypeptide has cellodextrin phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments, the recombinant polypeptide has cellobiose phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the recombinant polypeptide contains one or more mutations. In certain embodiments, the one or more mutations are amino acid substitutions. In certain embodiments that may be combined with any of the preceding embodiments, the recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof. In certain embodiments that may be combined with any of the preceding embodiments, the host cell further contains a recombinant phosphoglucomutase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the host cell further contains a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1.

Other aspects of the present disclosure relate to a host cell containing a recombinant cellodextrin transporter and a recombinant phosphoglucomutase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the host cell further contains a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1.

Other aspects of the present disclosure relate to a host cell containing a recombinant cellodextrin transporter and a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1. In certain embodiments that may be combined with any of the preceding embodiments, the host cell further contains a recombinant phosphoglucomutase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19).

In certain embodiments that may be combined with any of the preceding embodiments, the host cell further contains a recombinant polypeptide containing G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 14) or Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 15), where the recombinant polypeptide has cellodextrin phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments, the recombinant polypeptide has cellobiose phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the recombinant polypeptide contains one or more mutations. In certain embodiments, the one or more mutations are amino acid substitutions. In certain embodiments that may be combined with any of the preceding embodiments, the recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof. In certain embodiments that may be combined with any of the preceding embodiments, the host cell further contains a second recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the second recombinant polypeptide has β-glucosidase activity. In certain embodiments, the second recombinant polypeptide contains two or more sequences selected from F-x-[FYWM]-[GSTA]-x-[GSTA]-x-[GSTA](2)-[FYNH]-[NQ]-x-E-x-[GSTA] (SEQ ID NO: 16), [LIVMFSTC]-[LIVFYS]-[LIV]-[LIVMST]-E-N-G-[LIVMFAR]-[CSAGN] (SEQ ID NO: 17), and [LIVM](2)-[KR]-x-[EQKRD]-x(4)-G-[LIVMFTC]-[LIVT]-[LIVMF]-[ST]-D-x(2)-[SGADNIT] (SEQ ID NO: 18). In certain embodiments that may be combined with any of the preceding embodiments, the second recombinant polypeptide contains an amino acid sequence that is least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identical to the amino acid sequence of NCU00130.

Other aspects of the present disclosure relate to a host cell containing a recombinant cellodextrin transporter and a recombinant polypeptide containing one or more sequences selected from F-x-[FYWM]-[GSTA]-x-[GSTA]-x-[GSTA](2)-[FYNH]-[NQ]-x-E-x-[GSTA](SEQ ID NO: 18), [LIVMFSTC]-[LIVFYS]-[LIV]-[LIVMST]-E-N-G-[LIVMFAR]-[CSAGN](SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity. In certain embodiments, the recombinant polypeptide contains two or more sequences selected from F-x-[FYWM]-[GSTA]-x-[GSTA]-x-[GSTA](2)-[FYNH]-[NQ]-x-E-x-[GSTA] (SEQ ID NO: 16), [LIVMFSTC]-[LIVFYS]-[LIV]-[LIVMST]-E-N-G-[LIVMFAR]-[CSAGN] (SEQ ID NO: 17), and [LIVM](2)-[KR]-x-[EQKRD]-x(4)-G-[LIVMFTC]-[LIVT]-[LIVMF]-[ST]-D-x(2)-[SGADNIT] (SEQ ID NO: 18). In certain embodiments that may be combined with any of the preceding embodiments, the recombinant polypeptide contains an amino acid sequence that is least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identical to the amino acid sequence of NCU00130. In certain embodiments that may be combined with any of the preceding embodiments, the host cell further contains a recombinant phosphoglucomutase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the host cell further contains a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1. In certain embodiments that may be combined with any of the preceding embodiments, the host cell further contains a second recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the second recombinant polypeptide has cellodextrin phosphorylase activity. In certain embodiments, the second recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments, the second recombinant polypeptide has cellobiose phosphorylase activity. In certain embodiments, the second recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the second recombinant polypeptide contains one or more mutations. In certain embodiments, the one or more mutations are amino acid substitutions. In certain embodiments that may be combined with any of the preceding embodiments, the second recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof.

In certain embodiments that may be combined with any of the preceding embodiments, the recombinant cellodextrin transporter contains a polypeptide selected from a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 1 contains SEQ ID NO: 1; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 2 contains SEQ ID NO: 2; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and a loop connecting transmembrane α-helix 2 and transmembrane α-helix 3 contains SEQ ID NO: 3; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 5 contains SEQ ID NO: 4; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 6 contains SEQ ID NO: 5; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and sequence between transmembrane α-helix 6 and transmembrane α-helix 7 contains SEQ ID NO: 6; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 7 contains SEQ ID NO: 7; and a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 10 and transmembrane α-helix 11 and the sequence between them contain SEQ ID NO: 8. In certain embodiments, the recombinant cellodextrin transporter is a cellobiose transporter. In certain embodiments, the cellobiose transporter has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to SEQ ID NO: 9 (CDT-1) or SEQ ID NO: 10 (CDT-2). In certain embodiments that may be combined with any of the preceding embodiments, the recombinant cellodextrin transporter contains one or more mutations. In certain embodiments, the one or more mutations are amino acid substitutions. In certain embodiments that may be combined with any of the preceding embodiments, the recombinant cellodextrin transporter contains an amino acid substitution at one or more positions corresponding to positions of the amino acid sequence of SEQ ID NO: 9 (CDT-1), where the one or more amino acid substitutions are at positions selected from a position corresponding to amino acid 91 of SEQ ID NO: 9, a position corresponding to amino acid 104 of SEQ ID NO: 9, a position corresponding to amino acid 170 of SEQ ID NO: 9, a position corresponding to amino acid 174 of SEQ ID NO: 9, a position corresponding to amino acid 194 of SEQ ID NO: 9, a position corresponding to amino acid 213 of SEQ ID NO: 9, a position corresponding to amino acid 335 of SEQ ID NO: 9, and combinations thereof. In certain embodiments that may be combined with any of the preceding embodiments, the recombinant cellodextrin transporter contains an amino acid substitution at one or more positions corresponding to positions of the amino acid sequence of SEQ ID NO: 9 (CDT-1), where the one or more amino acid substitutions are at selected from a glycine (G) to alanine (A) substitution at a position corresponding to amino acid 91 of SEQ ID NO: 9, a glutamine (Q) to alanine (A) substitution at a position corresponding to amino acid 104 of SEQ ID NO: 9, a phenylalanie (F) to alanine (A) substitution at a position corresponding to amino acid 170 of SEQ ID NO: 9, an arginine (R) to alanine (A) substitution at a position corresponding to amino acid 174 of SEQ ID NO: 9, a glutamate (E) to alanine (A) substitution at a position corresponding to amino acid 194 of SEQ ID NO: 9, a phenylalanie (F) to lysine (L) substitution at a position corresponding to amino acid 213 of SEQ ID NO: 9, a phenylalanie (F) to alanine (A) substitution at a position corresponding to amino acid 335 of SEQ ID NO: 9, and combinations thereof.

Other aspects of the present disclosure relate to a host cell containing a recombinant phosphoglucomutase, and a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the host cell further contains a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1.

Other aspects of the present disclosure relate to a host cell containing a recombinant hexokinase, and a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1. In certain embodiments that may be combined with any of the preceding embodiments, the host cell further contains a recombinant phosphoglucomutase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments that may be combined with any of the preceding embodiments, the recombinant polypeptide has cellobiose phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the recombinant polypeptide contains one or more mutations. In certain embodiments, the one or more mutations are amino acid substitutions. In certain embodiments that may be combined with any of the preceding embodiments, the recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof.

Other aspects of the present disclosure relate to a host cell containing a recombinant phosphoglucomutase and a recombinant hexokinase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1. In certain embodiments that may be combined with any of the preceding embodiments, the host cell further contains a recombinant polypeptide containing G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 14) or Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 15), where the recombinant polypeptide has cellodextrin phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments, the recombinant polypeptide has cellobiose phosphorylase activity. In certain embodiments, the recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the recombinant polypeptide contains one or more mutations. In certain embodiments, the one or more mutations are amino acid substitutions. In certain embodiments that may be combined with any of the preceding embodiments, the recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof.

In certain embodiments that may be combined with any of the preceding embodiments, the host cell further contains a second recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the second recombinant polypeptide has β-glucosidase activity. In certain embodiments, the second recombinant polypeptide contains two or more sequences selected from F-x-[FYWM]-[GSTA]-x-[GSTA]-x-[GSTA](2)-[FYNH]-[NQ]-x-E-x-[GSTA] (SEQ ID NO: 16), [LIVMFSTC]-[LIVFYS]-[LIV]-[LIVMST]-E-N-G-[LIVMFAR]-[CSAGN] (SEQ ID NO: 17), and [LIVM](2)-[KR]-x-[EQKRD]-x(4)-G-[LIVMFTC]-[LIVT]-[LIVMF]-[ST]-D-x(2)-[SGADNIT] (SEQ ID NO: 18). In certain embodiments that may be combined with any of the preceding embodiments, the second recombinant polypeptide contains an amino acid sequence that is least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identical to the amino acid sequence of NCU00130.

Other aspects of the present disclosure relate to a host cell containing: a first recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the first recombinant polypeptide has cellodextrin phosphorylase activity, and a second recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the second recombinant polypeptide has β-glucosidase activity. In certain embodiments, the first recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments, the first recombinant polypeptide has cellobiose phosphorylase activity. In certain embodiments, the first recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the first recombinant polypeptide contains one or more mutations. In certain embodiments, the one or more mutations are amino acid substitutions. In certain embodiments that may be combined with any of the preceding embodiments, the first recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof. In certain embodiments that may be combined with any of the preceding embodiments, the host cell further contains a recombinant phosphoglucomutase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the host cell further contains a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1.

Other aspects of the present disclosure relate to a host cell containing a recombinant phosphoglucomutase, and a recombinant polypeptide containing one or more sequences selected from F-x-[FYWM]-[GSTA]-x-[GSTA]-x-[GSTA](2)-[FYNH]-[NQ]-x-E-x-[GSTA](SEQ ID NO: 18), [LIVMFSTC]-[LIVFYS]-[LIV]-[LIVMST]-E-N-G-[LIVMFAR]-[CSAGN](SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the host cell further contains a recombinant hexokinase. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1.

Other aspects of the present disclosure relate to a host cell containing a recombinant hexokinase, and a recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity. In certain embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1. In certain embodiments that may be combined with any of the preceding embodiments, the host cell further contains a recombinant phosphoglucomutase. In certain embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19).

In certain embodiments that may be combined with any of the preceding embodiments, the host cell further contains a second recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the second recombinant polypeptide has cellodextrin phosphorylase activity. In certain embodiments, the second recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments, the second recombinant polypeptide has cellobiose phosphorylase activity. In certain embodiments, the second recombinant polypeptide contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from SEQ ID NO: 11 (CgCBP), SEQ ID NO: 12 (SdCBP), and SEQ ID NO: 13 (CtCBP). In certain embodiments that may be combined with any of the preceding embodiments, the second recombinant polypeptide contains one or more mutations. In certain embodiments, the one or more mutations are amino acid substitutions. In certain embodiments that may be combined with any of the preceding embodiments, the second recombinant polypeptide contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof.

In certain embodiments that may be combined with any of the preceding embodiments, the host cell further contains a recombinant cellodextrin transporter containing a polypeptide selected from a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 1 contains SEQ ID NO: 1; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 2 contains SEQ ID NO: 2; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and a loop connecting transmembrane α-helix 2 and transmembrane α-helix 3 contains SEQ ID NO: 3; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 5 contains SEQ ID NO: 4; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 6 contains SEQ ID NO: 5; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and sequence between transmembrane α-helix 6 and transmembrane α-helix 7 contains SEQ ID NO: 6; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 7 contains SEQ ID NO: 7; and a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 10 and transmembrane α-helix 11 and the sequence between them contain SEQ ID NO: 8. In certain embodiments, the recombinant cellodextrin transporter is a cellobiose transporter. In certain embodiments, the cellobiose transporter has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to SEQ ID NO: 9 (CDT-1) or SEQ ID NO: 10 (CDT-2). In certain embodiments that may be combined with any of the preceding embodiments, the recombinant cellodextrin transporter contains one or more mutations. In certain embodiments, the one or more mutations are amino acid substitutions. In certain embodiments that may be combined with any of the preceding embodiments, the recombinant cellodextrin transporter contains an amino acid substitution at one or more positions corresponding to positions of the amino acid sequence of SEQ ID NO: 9 (CDT-1), where the one or more amino acid substitutions are at positions selected from a position corresponding to amino acid 91 of SEQ ID NO: 9, a position corresponding to amino acid 104 of SEQ ID NO: 9, a position corresponding to amino acid 170 of SEQ ID NO: 9, a position corresponding to amino acid 174 of SEQ ID NO: 9, a position corresponding to amino acid 194 of SEQ ID NO: 9, a position corresponding to amino acid 213 of SEQ ID NO: 9, a position corresponding to amino acid 335 of SEQ ID NO: 9, and combinations thereof. In certain embodiments that may be combined with any of the preceding embodiments, the recombinant cellodextrin transporter contains an amino acid substitution at one or more positions corresponding to positions of the amino acid sequence of SEQ ID NO: 9 (CDT-1), where the one or more amino acid substitutions are at selected from a glycine (G) to alanine (A) substitution at a position corresponding to amino acid 91 of SEQ ID NO: 9, a glutamine (Q) to alanine (A) substitution at a position corresponding to amino acid 104 of SEQ ID NO: 9, a phenylalanie (F) to alanine (A) substitution at a position corresponding to amino acid 170 of SEQ ID NO: 9, an arginine (R) to alanine (A) substitution at a position corresponding to amino acid 174 of SEQ ID NO: 9, a glutamate (E) to alanine (A) substitution at a position corresponding to amino acid 194 of SEQ ID NO: 9, a phenylalanie (F) to lysine (L) substitution at a position corresponding to amino acid 213 of SEQ ID NO: 9, a phenylalanie (F) to alanine (A) substitution at a position corresponding to amino acid 335 of SEQ ID NO: 9, and combinations thereof. In certain embodiments that may be combined with any of the preceding embodiments, the recombinant polypeptide having β-glucosidase activity contains two or more sequences selected from F-x-[FYWM]-[GSTA]-x-[GSTA]-x-[GSTA](2)-[FYNH]-[NQ]-x-E-x-[GSTA] (SEQ ID NO: 16), [LIVMFSTC]-[LIVFYS]-[LIV]-[LIVMST]-E-N-G-[LIVMFAR]-[CSAGN] (SEQ ID NO: 17), and [LIVM](2)-[KR]-x-[EQKRD]-x(4)-G-[LIVMFTC]-[LIVT]-[LIVMF]-[ST]-D-x(2)-[SGADNIT] (SEQ ID NO: 18). In certain embodiments, the recombinant polypeptide having β-glucosidase activity contains an amino acid sequence that is least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identical to the amino acid sequence of NCU00130.

In certain embodiments that may be combined with any of the preceding embodiments, the host cell further contains one or more glucose response genes, where the activity level of a protein encoded by at least one glucose response gene is altered compared to the wild-type activity level of the protein. In certain embodiments, the one or more glucose response genes are selected from Snf3, Rgt1, Rgt2, Yck1/2, Std1, Mthy1, Snf1/4, Grr1, Gpr1, Gpa2, Ras2, Stb3, Hxk2, Pfk27, Pfk26, Sch9, Yak1, Mig1, Rim15, Kcs1, and Tps1. In certain embodiments that may be combined with any of the preceding embodiments, the activity level of the protein encoded by at least one glucose response gene is increased compared to its wild-type activity level. In certain embodiments that may be combined with any of the preceding embodiments, the activity level of the protein encoded by at least one glucose response gene is decreased compared to its wild-type activity level. In certain embodiments that may be combined with any of the preceding embodiments, the host cell is a fungal cell. In certain embodiments that may be combined with any of the preceding embodiments, the host cell is a yeast cell. In certain embodiments, the yeast cell is S. cerevisiae.

Certain aspects of the present disclosure relate to a method for degrading cellodextrin, by: a) providing a host cell containing two or more of: a recombinant cellodextrin transporter, a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity, a recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity, a recombinant phosphoglucomutase, and a recombinant hexokinase; and b) culturing the host cell in a medium containing cellodextrin or a source of cellodextrin, whereby cellodextrin is transported into the cell and degraded. Other aspects of the present disclosure relate to a method for producing hydrocarbons or hydrocarbon derivatives from cellodextrin, by: a) providing a host cell containing two or more of: a recombinant cellodextrin transporter, a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity, a recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity, a recombinant phosphoglucomutase, and a recombinant hexokinase; and b) culturing the host cell in a medium containing cellodextrin or a source of cellodextrin, whereby cellodextrin is transported into the cell and degraded and whereby the host cell produces hydrocarbons or hydrocarbon derivatives from the cellodextrin. In certain embodiments that may be combined with any of the preceding embodiments, the host cell contains two or more, three or more, or four of: a recombinant cellodextrin transporter, a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity, a recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity, a recombinant phosphoglucomutase, and a recombinant hexokinase.

In certain embodiments that may be combined with any of the preceding embodiments, the recombinant polypeptide having cellodextrin phosphorylase activity contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments that may be combined with any of the preceding embodiments, the recombinant polypeptide having cellodextrin phosphorylase activity is a cellobiose phosphorylase. In certain embodiments, the cellobiose phosphorylase contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid selected from SEQ ID NO: 11 (CgCBP), SEQ ID NO: 12 (SdCBP), and SEQ ID NO: 13 (CtCBP). In certain embodiments that may be combined with any of the preceding embodiments, the recombinant polypeptide having cellodextrin phosphorylase activity contains one or more mutations. In certain embodiments, the one or more mutations are amino acid substitutions. In certain embodiments that may be combined with any of the preceding embodiments, the recombinant polypeptide having cellodextrin phosphorylase activity contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof. In certain embodiments that may be combined with any of the preceding embodiments, the recombinant polypeptide having β-glucosidase activity contains two or more sequences selected from F-x-[FYWM]-[GSTA]-x-[GSTA]-x-[GSTA](2)-[FYNH]-[NQ]-x-E-x-[GSTA] (SEQ ID NO: 16), [LIVMFSTC]-[LIVFYS]-[LIV]-[LIVMST]-E-N-G-[LIVMFAR]-[CSAGN] (SEQ ID NO: 17), and [LIVM](2)-[KR]-x-[EQKRD]-x(4)-G-[LIVMFTC]-[LIVT]-[LIVMF]-[ST]-D-x(2)-[SGADNIT] (SEQ ID NO: 18). In certain embodiments that may be combined with any of the preceding embodiments, the recombinant polypeptide having β-glucosidase activity contains an amino acid sequence that is least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identical to the amino acid sequence of NCU00130. In certain embodiments that may be combined with any of the preceding embodiments, the recombinant polypeptide having cellodextrin phosphorylase activity reduces ATP consumption as compared to a corresponding cell lacking the recombinant polypeptide having cellodextrin phosphorylase activity. In certain embodiments that may be combined with any of the preceding embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1. In certain embodiments that may be combined with any of the preceding embodiments, the recombinant cellodextrin transporter contains a polypeptide selected from a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 1 contains SEQ ID NO: 1; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 2 contains SEQ ID NO: 2; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and a loop connecting transmembrane α-helix 2 and transmembrane α-helix 3 contains SEQ ID NO: 3; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 5 contains SEQ ID NO: 4; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 6 contains SEQ ID NO: 5; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and sequence between transmembrane α-helix 6 and transmembrane α-helix 7 contains SEQ ID NO: 6; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 7 contains SEQ ID NO: 7; and a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 10 and transmembrane α-helix 11 and the sequence between them contains SEQ ID NO: 8. In certain embodiments, the recombinant cellodextrin transporter is a cellobiose transporter. In certain embodiments, the cellobiose transporter has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to SEQ ID NO: 9 (CDT-1) or SEQ ID NO: 10 (CDT-2). In certain embodiments that may be combined with any of the preceding embodiments, the recombinant cellodextrin transporter contains one or more mutations. In certain embodiments, the one or more mutations are amino acid substitutions. In certain embodiments that may be combined with any of the preceding embodiments, the recombinant cellodextrin transporter contains an amino acid substitution at one or more positions corresponding to positions of the amino acid sequence of SEQ ID NO: 9 (CDT-1), where the one or more positions are at positions selected from a position corresponding to amino acid 91 of SEQ ID NO: 9, a position corresponding to amino acid 104 of SEQ ID NO: 9, a position corresponding to amino acid 170 of SEQ ID NO: 9, a position corresponding to amino acid 174 of SEQ ID NO: 9, a position corresponding to amino acid 194 of SEQ ID NO: 9, a position corresponding to amino acid 213 of SEQ ID NO: 9, a position corresponding to amino acid 335 of SEQ ID NO: 9, and combinations thereof. In certain embodiments that may be combined with any of the preceding embodiments, the recombinant cellodextrin transporter contains an amino acid substitution at one or more positions corresponding to positions of the amino acid sequence of SEQ ID NO: 9 (CDT-1), where the one or more amino acid substitutions are selected from a glycine (G) to alanine (A) substitution at a position corresponding to amino acid 91 of SEQ ID NO: 9, a glutamine (Q) to alanine (A) substitution at a position corresponding to amino acid 104 of SEQ ID NO: 9, a phenylalanie (F) to alanine (A) substitution at a position corresponding to amino acid 170 of SEQ ID NO: 9, an arginine (R) to alanine (A) substitution at a position corresponding to amino acid 174 of SEQ ID NO: 9, a glutamate (E) to alanine (A) substitution at a position corresponding to amino acid 194 of SEQ ID NO: 9, a phenylalanie (F) to lysine (L) substitution at a position corresponding to amino acid 213 of SEQ ID NO: 9, a phenylalanie (F) to alanine (A) substitution at a position corresponding to amino acid 335 of SEQ ID NO: 9, and combinations thereof. In certain embodiments that may be combined with any of the preceding embodiments, the host cell further contains one or more glucose response genes, where the activity level of a protein encoded by at least one glucose response gene is altered compared to the wild-type activity level of the protein. In certain embodiments, the one or more glucose response genes are selected from Snf3, Rgt1, Rgt2, Yck1/2, Std1, Mthy1, Snf1/4, Grr1, Gpr1, Gpa2, Ras2, Stb3, Hxk2, Pfk27, Pfk26, Sch9, Yak1, Mig1, Rim15, Kcs1, and Tps1. In certain embodiments that may be combined with any of the preceding embodiments, the activity level of one or more proteins encoded by the one or more glucose response genes is increased compared to its wild-type activity level. In certain embodiments that may be combined with any of the preceding embodiments, the activity level of one or more proteins encoded by the one or more glucose response genes is decreased compared to its wild-type activity level. In certain embodiments that may be combined with any of the preceding embodiments, the source of cellodextrin contains cellulose. In certain embodiments that may be combined with any of the preceding embodiments, the cellodextrin is selected from cellobiose, cellotriose, cellotetraose, cellopentose, and cellohexose. In certain embodiments that may be combined with any of the preceding embodiments, the hydrocarbons or hydrocarbon derivatives can be used as fuel. In certain embodiments that may be combined with any of the preceding embodiments, the hydrocarbons or hydrocarbon derivatives contain ethanol. In certain embodiments, the ethanol is produced at a rate that ranges from at least about 0.10 to at least 20 g/L-h. In certain embodiments that may be combined with any of the preceding embodiments, the hydrocarbons or hydrocarbon derivatives contain butanol. In certain embodiments that may be combined with any of the preceding embodiments, the host cell is a fungal cell. In certain embodiments that may be combined with any of the preceding embodiments, the host cell is a yeast cell. In certain embodiments, the yeast cell is S. cerevisiae.

Other aspects of the present disclosure relate to a host cell containing two or more of: a recombinant cellodextrin transporter, a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS](SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity, a recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT][LIVMF]-[ST]-D-x(2)-[SGADNIT] (SEQ ID NO: 20), where the recombinant polypeptide has β-glucosidase activity, a recombinant phosphoglucomutase, and a recombinant hexokinase. In certain embodiments, the host cell contains two or more, three or more, or four of: a recombinant cellodextrin transporter, a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity, a recombinant polypeptide containing one or more sequences selected from F-x-[FYWM]-[GSTA]-x-[GSTA]-x-[GSTA](2)-[FYNH]-[NQ]-x-E-x-[GSTA] (SEQ ID NO: 18), [LIVMFSTC]-[LIVFYS]-[LIV]-[LIVMST]-E-N-G-[LIVMFAR]-[CSAGN] (SEQ ID NO: 19), and [LIVM](2)-[KR]-x-[EQKRD]-x(4)-G-[LIVMFTC]-[LIVT]-[LIVMF]-[ST]-D-x(2)-[SGADNIT] (SEQ ID NO: 20), where the recombinant polypeptide has β-glucosidase activity, a recombinant phosphoglucomutase, and a recombinant hexokinase.

In certain embodiments, the recombinant polypeptide having cellodextrin phosphorylase activity contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell. In certain embodiments, the recombinant polypeptide having cellodextrin phosphorylase activity is a cellobiose phosphorylase. In certain embodiments, the recombinant cellobiose phosphorylase contains an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid selected from SEQ ID NO: 11 (CgCBP), SEQ ID NO: 12 (SdCBP), and SEQ ID NO: 13 (CtCBP). In certain embodiments that may be combined with any of the preceding embodiments, the recombinant polypeptide having cellodextrin phosphorylase activity contains one or more mutations. In certain embodiments, the one or more mutations are amino acid substitutions. In certain embodiments that may be combined with any of the preceding embodiments, the recombinant polypeptide having cellodextrin phosphorylase activity contains an amino acid substitution at one or more positions corresponding to positions of the amino acid sequence of SEQ ID NO: 12 (SdCBP), where the one or more amino acid substitutions are selected from an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof. In certain embodiments that may be combined with any of the preceding embodiments, the recombinant polypeptide having β-glucosidase activity contains two or more sequences selected from F-x-[FYWM]-[GSTA]-x-[GSTA]-x-[GSTA](2)-[FYNH]-[NQ]-x-E-x-[GSTA] (SEQ ID NO: 16), [LIVMFSTC]-[LIVFYS]-[LIV]-[LIVMST]-E-N-G-[LIVMFAR]-[CSAGN] (SEQ ID NO: 17), and [LIVM](2)-[KR]-x-[EQKRD]-x(4)-G-[LIVMFTC]-[LIVT]-[LIVMF]-[ST]-D-x(2)-[SGADNIT] (SEQ ID NO: 18). In certain embodiments that may be combined with any of the preceding embodiments, the recombinant polypeptide having β-glucosidase contains an amino acid sequence that is least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identical to the amino acid sequence of NCU00130. In certain embodiments that may be combined with any of the preceding embodiments, the recombinant phosphoglucomutase contains a conserved motif having the amino acid sequence of [GSA]-[LIVMF]-x-[LIVM]-[ST]-[PGA]-S-H-[NIC]-P (SEQ ID NO: 19). In certain embodiments that may be combined with any of the preceding embodiments, the recombinant hexokinase contains a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20). In certain embodiments, the recombinant hexokinase is HXK1. In certain embodiments that may be combined with any of the preceding embodiments, the recombinant cellodextrin transporter contains a polypeptide selected from a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 1 contains SEQ ID NO: 1; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 2 contains SEQ ID NO: 2; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and a loop connecting transmembrane α-helix 2 and transmembrane α-helix 3 contains SEQ ID NO: 3; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 5 contains SEQ ID NO: 4; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 6 contains SEQ ID NO: 5; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and sequence between transmembrane α-helix 6 and transmembrane α-helix 7 contains SEQ ID NO: 6; a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 7 contains SEQ ID NO: 7; and a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 10 and transmembrane α-helix 11 and the sequence between them contains SEQ ID NO: 8. In certain embodiments, the recombinant cellodextrin transporter is a cellobiose transporter. In certain embodiments, the cellobiose transporter has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to SEQ ID NO: 9 (CDT-1) or SEQ ID NO: 10 (CDT-2). In certain embodiments that may be combined with any of the preceding embodiments, the recombinant cellodextrin transporter contains one or more mutations. In certain embodiments, the one or more mutations are amino acid substitutions. In certain embodiments that may be combined with any of the preceding embodiments, the recombinant cellodextrin transporter contains an amino acid substitution at one or more positions corresponding to positions of the amino acid sequence of SEQ ID NO: 9 (CDT-1), where the one or more amino acid substitutions are at positions selected from a position corresponding to amino acid 91 of SEQ ID NO: 9, a position corresponding to amino acid 104 of SEQ ID NO: 9, a position corresponding to amino acid 170 of SEQ ID NO: 9, a position corresponding to amino acid 174 of SEQ ID NO: 9, a position corresponding to amino acid 194 of SEQ ID NO: 9, a position corresponding to amino acid 213 of SEQ ID NO: 9, a position corresponding to amino acid 335 of SEQ ID NO: 9, and combinations thereof. In certain embodiments that may be combined with any of the preceding embodiments, the recombinant cellodextrin transporter contains an amino acid substitution at one or more positions corresponding to positions of the amino acid sequence of SEQ ID NO: 9 (CDT-1), where the one or more amino acid substitutions are at positions selected from a glycine (G) to alanine (A) substitution at a position corresponding to amino acid 91 of SEQ ID NO: 9, a glutamine (Q) to alanine (A) substitution at a position corresponding to amino acid 104 of SEQ ID NO: 9, a phenylalanie (F) to alanine (A) substitution at a position corresponding to amino acid 170 of SEQ ID NO: 9, an arginine (R) to alanine (A) substitution at a position corresponding to amino acid 174 of SEQ ID NO: 9, a glutamate (E) to alanine (A) substitution at a position corresponding to amino acid 194 of SEQ ID NO: 9, a phenylalanie (F) to lysine (L) substitution at a position corresponding to amino acid 213 of SEQ ID NO: 9, a phenylalanie (F) to alanine (A) substitution at a position corresponding to amino acid 335 of SEQ ID NO: 9, and combinations thereof. In certain embodiments that may be combined with any of the preceding embodiments, the host cell further contains one or more glucose response genes, where the activity level of a protein encoded by at least one glucose response gene is altered compared to the wild-type activity level of the protein. In certain embodiments, the one or more glucose response genes are selected from Snf3, Rgt1, Rgt2, Yck1/2, Std1, Mthy1, Snf1/4, Grr1, Gpr1, Gpa2, Ras2, Stb3, Hxk2, Pfk27, Pfk26, Sch9, Yak1, Mig1, Rim15, Kcs1, and Tps1. In certain embodiments that may be combined with any of the preceding embodiments, the activity level of the protein encoded by at least one glucose response gene is increased compared to its wild-type activity level. In certain embodiments that may be combined with any of the preceding embodiments, the activity level of the protein encoded by at least one glucose response gene is decreased compared to its wild-type activity level. In certain embodiments that may be combined with any of the preceding embodiments, the host cell is a fungal cell. In certain embodiments that may be combined with any of the preceding embodiments, the host cell is a yeast cell. In certain embodiments, the yeast cell is S. cerevisiae.

### BRIEF DESCRIPTION OF THE DRAWINGS

FIG. 1 shows a comparison between metabolic pathways presently in yeast and a novel metabolic pathway of the present disclosure. The standard metabolic pathway in yeast is shown to the left of the hatched line. Here, glucose enters the cell through hexose transporters and is then phosphorylated by hexokinase to form glucose-6-phosphate. The novel metabolic pathway of the present disclosure is shown to the right of the hatched line. Here, cellodextrins enter the cell through cellodextrin transporters. One glucose moiety is subsequently cleaved off by a cellodextrin phosphorylase resulting in glucose-1-phosphate and a shortened cellodextrin. The glucose-1-phosphate is converted to glucose-6-phosphate by phosphoglucomutase. FIG. 1 shows the scheme when cellotriose is used as a carbon source.

FIG. 2 schematically shows two possible cellobiose fermentation pathways. Following transport across the plasma membrane by a cellodextrin transporter (CDT), cellobiose is cleaved either by hydrolysis (left panel) via a β-glucosidase (e.g., GH1) or by phosphorolysis (right panel) via a cellobiose phosphorylase (e.g., CBP). Intracellular glucose is formed in the hydrolytic pathway and is converted to glucose-6-phosphate (Glc6P) by hexokinase (HXK). Intracellular glucose and glucose-1-phosphate are formed in the phosphorolytic pathway. Here, glucose-1-phosphate is converted to Glc6P by phosphoglucomutase (PGM), while glucose is converted to Glc6P by HXK. In both pathways Glc6P is fermented to ethanol and CO₂ by endogenous yeast enzymes. Enzymes engineered into yeast are italicized.

FIG. 3 depicts the cellobiose fermentation profile of three engineered yeast strains each expressing the cellodextrin transporter gene cdt-1, and one of three cellobiose phosphorylase genes. The rate at which the strains fermented cellobiose (triangles) to ethanol (diamonds) was determined under oxygen limited conditions. The amount of produced biomass was also monitored (circles). Each profile was run independently duplicated, and there was less than 5% variance between replicates. Shown is a representative profile. FIG. 3A depicts the D452-2 strain of S. cerevisiae, transformed with cdt-1, and a codon-optimized cellobiose phosphorylase gene from C. gilvus. FIG. 3B depicts the D452-2 strain of S. cerevisiae, transformed with cdt-1, and a codon-optimized cellobiose phosphorylase gene from S. degradans. FIG. 3C depicts the D452-2 strain of S. cerevisiae, transformed with cdt-1, and a codon-optimized cellobiose phosphorylase gene from C. thermocellum.

FIG. 4 depicts the cellobiose fermentation profile of three engineered yeast strains each expressing the S. cerevisiae gene for PGM, along with the cellodextrin transporter gene cdt-1 and a cellobiose phosphorylase gene. The rate at which the strains fermented cellobiose (triangles) to ethanol (diamonds) was determined under oxygen limited conditions. The amount of produced biomass was also monitored (circles). Each profile was run independently duplicated, and there was less than 5% variance between replicates.

FIG. 5 depicts the fermentation profile of engineered yeast strains bearing a spontaneously derived mutant of the cellodextrin transporter gene cdt-1. The rate at which the strains fermented cellobiose (triangles) to ethanol (diamonds) was determined under oxygen limited conditions. The amount of produced biomass was also monitored (circles). All values are the means of the results for two independent fermentations, and error bars represent the standard deviations of the results between two fermentations. FIG. 5A depicts the D452-2 strain of S. cerevisiae transformed with WT cdt-1 and a codon-optimized cellobiose phosphorylase gene from S. degradans. FIG. 5B depicts the D452-2 strain of S. cerevisiae transformed with a mutant cdt-1 (F213L) and a codon-optimized cellobiose phosphorylase gene from S. degradans. FIG. 5C depicts the D452-2 strain of S. cerevisiae transformed with WT cdt-1 and the β-glucosidase gene gh1-1. FIG. 5D depicts the D452-2 strain of S. cerevisiae transformed with a mutant cdt-1 (F213L) and the β-glucosidase gene gh1-1.

FIG. 6A deceits time profiles of cellobiose fermentation with various mutant CDT-1 via the hydrolytic pathway. FIG. 6B depicts time profiles of cellobiose fermentation with various mutant CDT-1 via the phosphorolytic pathway.

FIG. 7 depicts cellobiose consumption and ethanol production of engineered yeast strains with various cellodextrin transporter gene (cdt-1) mutants (G91A, Q104A, F170A, R174A, E194A, F213L, and F335A). The D452-2 strain of S. cerevisiae was transformed with either WT cdt-1 or a cdt-1 mutant, and a codon-optimized cellobiose phosphorylase gene from S. degradans or the β-glucosidase gene gh1-1. Productivities with various cdt-1 mutants and cellobiose phosphorylase are depicted along the y-axis, while productivities with various cdt-1 mutants and β-glucosidase are depicted along the x-axis. All values are the means of the results for two independent fermentations, and error bars represent the standard deviations of the results between two fermentations. FIG. 7A depicts the rate of cellobiose consumption. FIG. 7B depicts the rate of ethanol production.

FIG. 8 depicts the transport kinetics of WT cdt-1 and three cdt-1 mutants. The linear rate of [³H] cellobiose uptake into engineered yeast strains expressing WT cdt-1, cdt-1 (G91A), cdt-1 (F335A), or cdt-1 (F213L), was determined using various concentrations of cellobiose. Error bars represent the standard deviation of three replicate measurements at each concentration. FIG. 8A depicts the transport kinetics of WT cdt-1. FIG. 8B depicts the transport kinetics of cdt-1 (G91A). FIG. 8C depicts the transport kinetics of cdt-1 (F335A). FIG. 8D depicts the transport kinetics of cdt-1 (F213L).

FIG. 9 depicts the correlation between expression levels of CDT-1 mutants and fermentation performance. Cellobiose was fermented to ethanol by engineered yeast strains with one GFP-tagged variant of CDT-1 and either the β-glucosidase GH1-1 or the cellobiose phosphorylase SdCBP. During the exponential phase of these fermentations, cells were harvested and GFP fluorescence measured and plotted after correcting for culture OD. Values shown are the mean of two fermentations and error bars represent the standard deviation between two fermentations. FIG. 9A depicts engineered yeast strains with one GFP-tagged variant of CDT-1 and the β-glucosidase GH1-1. FIG. 9B depicts engineered yeast strains with one GFP-tagged variant of CDT-1 and the cellobiose phosphorylase SdCBP.

FIG. 10 depicts the activity of purified β-glucosidase GH1-1, S. degradans cellobiose phosphorylase SdCBP, and S. cerevisiae hexokinases in cell extracts. Cell extracts were harvested from D452-2 yeast growing on rich glucose media (YPD80), and from engineered D452-2 yeast expressing the cellodextrin transporter CDT-1 and either the β-glucosidase GH1-1 or the S. degradans cellobiose phosphorylase SdCBP growing on rich cellobiose media (YPC80). The amount of hexokinase activity, or cellobiase activity (defined as the rate of glucose released regardless of the mechanism) in 10 μg of extract was determined. In addition the amount of transporter present in each strain was estimated by measuring GFP fluorescence. Results are the mean+/−the standard deviation of three cultures. FIG. 10A depicts transporter abundance. FIG. 10B depicts cellobiose activity. FIG. 10C depicts hexokinase activity.

FIG. 11 depicts transglycosylation activity of the β-glucosidase GH1-1. 200 pkat of either purified GH1-1 was incubated with 20% (w/v) cellobiose for 24 hours at 30° C. in 50 mM phosphate buffer, pH 6.0. The same incubation was carried out without the addition of enzyme as a control. The products were then analyzed by HPLC.

FIG. 12 depicts the characteristics of purified GH-1 and SdCBP enzymes. The β-glucosidase GH1-1 and the S. degradans cellobiose phosphorylase SdCBP were purified directly from the yeast strains being studied. Linear rates of catalysis were determined at a variety of cellobiose concentrations in triplicate, and kinetic parameters determined by non-linear regression. FIG. 12A depicts the kinetics of GH-1. FIG. 12B depicts the kinetics of SdCBP.

FIG. 13 depicts the effect of cellobiose on the activity of purified hexokinases. The three S. cerevisiae hexokinases Hxk1, Hxk2, and Glk1 were expressed and purified from E. coli. To determine the effect of cellobiose on hexokinase activity, linear rates of glucose-phosphorylating activity were determined in the presence (grey bars) or absence (black bars) of 184 mM cellobiose. Results are the mean+/−the standard deviation of triplicate measurements.

FIG. 14 depicts comparisons of the over-expression of the hexokinases HXK1, HXK2, and GLK1 for improved cellobiose fermentation capability with mutant cellodextrin transporter CDT-1 (F213L) via the phosphorolytic pathway. FIG. 14A depicts yeast cell density. FIG. 14B depicts cellobiose consumption. FIG. 14C depicts ethanol production.

FIG. 15 depicts the profile of cellobiose fermentation via the phosphorolytic pathway of an engineered yeast strain over-expressing the hexokinase HXK1 and the cellodextrin transporter mutant CDT-1 (F213L) when cultured at four different initial cell OD values. Cellobiose consumption (squares), ethanol production (diamonds), and yeast cell density (circles) were measured. FIG. 15A depicts an initial OD of 1.6. FIG. 15B depicts an initial OD of 7.5. FIG. 15C depicts an initial OD of 13.6. FIG. 15D depicts an initial OD of 23.1.

FIG. 16 depicts the linear relationship between initial cell OD and ethanol productivity via the phosphorolytic pathway when the hexokinase HXK1 is over-expressed with the mutant cellodextrin CDT-1 (F213L).

FIG. 17 depicts the crystal structure of the Cellvibrio gilvus cellobiose phosphorylase (PDB 2CQS). The identified motif is in dark grey.

FIG. 18 depicts growth curves of engineered S. cerevisiae strains grown on cellobiose, cellotriose, and cellotetraose. Symbols: S. cerevisiae D452-2 (), D452-SdCBP-CDT-1 (∇), D452-CDP_Acell-CDT-1 (▪), D452-CDP_Clent-CDT-1 (⋄), and D452-CDP_Ctherm-CDT-1 (▴). FIG. 18A depicts growth on cellobiose. FIG. 18B depicts growth on cellotriose. FIG. 18C depicts growth on cellotetraose.

FIG. 19 depicts growth curves of engineered S. cerevisiae strains grown on cellobiose, cellotriose, and cellotetraose. Symbols: S. cerevisiae D452-2 (), D452-SdCBP-CDT-1_F213L (∇), D452-CDP_Acell-CDT-1_F213L (▪), D452-CDP_Clent-CDT-1_F213L (⋄), and D452-CDP_Ctherm-CDT-1_F213L (▴). FIG. 19A depicts growth on cellobiose. FIG. 19B depicts growth on cellotriose. FIG. 19C depicts growth on cellotetraose.

FIG. 20 depicts the growth of cellobiose-utilizing S. cerevisiae strains containing single deletions of genes involved in sensing of extracellular sugar. FIG. 20 depicts growth on cellobiose and glucose of a wild-type cellobiose-utilizing S. cerevisiae strain (WT), a cellobiose-utilizing S. cerevisiae strain containing an Snf3 deletion (ΔSnf3), a cellobiose-utilizing S. cerevisiae strain containing an Rgt2 deletion (ΔRgt2), and a cellobiose-utilizing S. cerevisiae strain containing a Gpr1 deletion (ΔGpr1).

FIG. 21 depicts the growth of cellobiose-utilizing S. cerevisiae strains containing single deletions of genes involved in intracellular signaling pathways. FIG. 21A depicts growth on cellobiose and glucose of a wild-type cellobiose-utilizing S. cerevisiae strain (WT), a cellobiose-utilizing S. cerevisiae strain containing a Gpa2 deletion (ΔGpa2), and a cellobiose-utilizing S. cerevisiae strain containing a Ras2 deletion (ΔRas2). Gpa2 and Ras2 function in parallel to activate the cAMP-dependent Protein Kinase A (PKA) pathway. FIG. 21B depicts growth on cellobiose and glucose of a wild-type cellobiose-utilizing S. cerevisiae strain (WT), a cellobiose-utilizing S. cerevisiae strain containing a Ras2 deletion (ΔRas2), and a cellobiose-utilizing S. cerevisiae strain containing an Sch9 deletion (ΔSch9). Sch9 acts in parallel to the Ras/PKA pathway. FIG. 21C depicts growth on cellobiose and glucose of a wild-type cellobiose-utilizing S. cerevisiae strain (WT), and a cellobiose-utilizing S. cerevisiae strain containing a Yak1 deletion (ΔYak1). Yak1 is a protein kinase that works in parallel to the Ras/PKA pathway but inhibits rather than stimulates cell growth.

FIG. 22 depicts growth on cellobiose and glucose of a wild-type cellobiose-utilizing S. cerevisiae strain (WT), a cellobiose-utilizing S. cerevisiae strain containing a Gpa2 deletion (ΔGpa2), and a cellobiose-utilizing S. cerevisiae strain containing the Gpa2 G132V mutant [Gpa2 (G132V)].

FIG. 23A depicts growth on cellobiose and glucose of a wild-type cellobiose-utilizing S. cerevisiae strain (WT), and a cellobiose-utilizing S. cerevisiae strain containing an Hxk2 deletion (ΔKxk2). FIG. 23B depicts growth on cellobiose and glucose of a wild-type cellobiose-utilizing S. cerevisiae strain (WT), and a cellobiose-utilizing S. cerevisiae strain containing the Hxk2wrf mutant.

FIG. 24 depicts growth on cellobiose and glucose of a wild-type cellobiose-utilizing S. cerevisiae strain (WT), a cellobiose-utilizing S. cerevisiae strain containing a Rim15 deletion (ΔRim15), a cellobiose-utilizing S. cerevisiae strain containing an Stb3 deletion (ΔStb3), and a cellobiose-utilizing S. cerevisiae strain containing a Kcs1 deletion (ΔKcs1).

### DETAILED DESCRIPTION

### Overview

The present disclosure relates to host cells containing two or more of a recombinant cellodextrin transporter, a recombinant cellodextrin phosphorylase, a recombinant β-glucosidase, a recombinant phosphoglucomutase, or a recombinant hexokinase. Other aspects of the present disclosure relate to methods for degrading cellodextrin, by providing a host cell containing two or more of a recombinant cellodextrin transporter, a recombinant cellodextrin phosphorylase, a recombinant β-glucosidase, a recombinant phosphoglucomutase, or a recombinant hexokinase; and culturing the host cell in a medium containing cellodextrin or a source of cellodextrin, whereby cellodextrin degraded. Still other aspects of the present disclosure relate to methods for producing hydrocarbons or hydrocarbon derivatives from cellodextrin, by providing a host cell containing two or more of a recombinant cellodextrin transporter, a recombinant cellodextrin phosphorylase, a recombinant β-glucosidase, a recombinant phosphoglucomutase, or a recombinant hexokinase; and culturing the host cell in a medium containing cellodextrin or a source of cellodextrin, whereby the host cell produces hydrocarbons or hydrocarbon derivatives from the cellodextrin. Further aspects of the present disclosure relate to methods for reducing ATP consumption during glucose utilization, by providing a host cell containing a recombinant cellodextrin phosphorylase and one or more of a recombinant cellodextrin transporter, a recombinant phosphoglucomutase, or a recombinant hexokinase; and culturing the host cell in a medium containing cellodextrin or a source of cellodextrin, whereby cellodextrin is degraded by the recombinant cellodextrin phosphorylase to glucose-1-phosphate, where the production of glucose-1-phosphate from cellodextrin reduces ATP consumption as compared to a corresponding cell lacking the recombinant polypeptide.

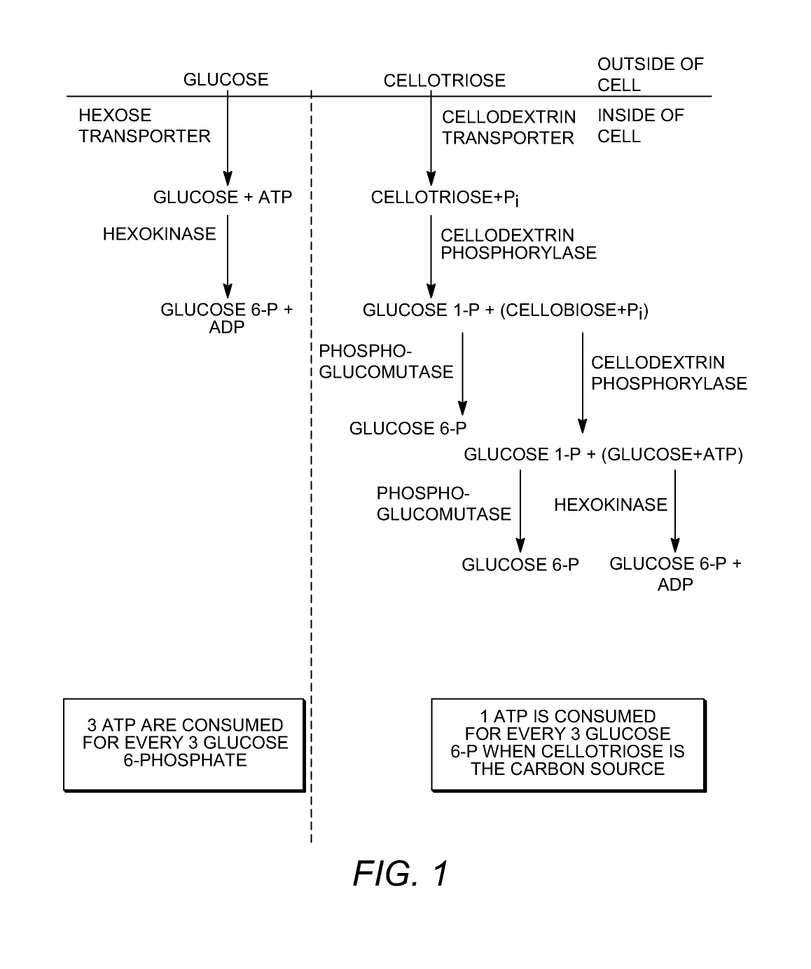

Moreover, the present disclosure is based at least in part on a novel strategy for degrading cellodextrin to phosphorylated glucose by utilizing an S. cerevisiae strain that was engineered to express the Neurospora crassa cellodextrin transporter gene cdt-1 to transport cellodextrin into the cell; a cellobiose phosphorylase gene from Celvibrio gilvus, Sacharophagus degradans, or Clostridium thermocellum to degrade the transported cellodextrin to glucose-1-phosphate and glucose; a recombinant phosphoglucomutase to convert the glucose-1-phosphate to glucose-6-phosphate; and a recombinant hexokinase to convert the glucose produced by the degradation of cellodextrin to glucose-6-phosphate (FIG. 1).

### DEFINITIONS

Unless defined otherwise, all scientific and technical terms are understood to have the same meaning as commonly used in the art to which they pertain. For the purpose of the present disclosure, the following terms are defined.

As used herein, “cellodextrin” refers to a β(1→4) glucose polymers of varying length and includes, without limitation, cellobiose (2 glucose monomers), cellotriose (3 glucose monomers), cellotetraose (4 glucose monomers), cellopentaose (5 glucose monomers), and cellohexaose (6 glucose monomers).

As used herein, a “cellodextrin phosphorylase” refers to an enzyme that catalyzes the degradation of a cellodextrin by utilizing inorganic phosphate to cleave one or more β-glucosidic linkages between glucose moieties in the cellodextrin.

As used herein, a “cellodextrin transporter” refers to any sugar transport protein capable of transporting cellodextrins across the cell membrane of a cell.

As used herein, “sugar” refers to monosaccharides (e.g., glucose, fructose, galactose, xylose, arabinose), disaccharides (e.g., cellobiose, sucrose, lactose, maltose), and oligosaccharides (typically containing 3 to 10 component monosaccharides).

As used herein, the terms “polynucleotide,” “nucleic acid sequence,” “sequence of nucleic acids,” and variations thereof shall be generic to polydeoxyribonucleotides (containing 2-deoxy-D-ribose), to polyribonucleotides (containing D-ribose), to any other type of polynucleotide that is an N-glycoside of a purine or pyrimidine base, and to other polymers containing non-nucleotidic backbones, provided that the polymers contain nucleobases in a configuration that allows for base pairing and base stacking, as found in DNA and RNA. Thus, these terms include known types of nucleic acid sequence modifications, for example, substitution of one or more of the naturally occurring nucleotides with an analog; inter-nucleotide modifications, such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalkylphosphoramidates, aminoalkylphosphotriesters); those containing pendant moieties, such as, for example, proteins (including nucleases, toxins, antibodies, signal peptides, poly-L-lysine, etc.); those with intercalators (e.g., acridine, psoralen, etc.); and those containing chelators (e.g., metals, radioactive metals, boron, oxidative metals, etc.). As used herein, the symbols for nucleotides and polynucleotides are those recommended by the IUPAC-IUB Commission of Biochemical Nomenclature (Biochem. 9:4022, 1970).

As used herein, a “polypeptide” is an amino acid sequence containing a plurality of consecutive polymerized amino acid residues (e.g., at least about 15 consecutive polymerized amino acid residues, optionally at least about 30 consecutive polymerized amino acid residues, at least about 50 consecutive polymerized amino acid residues). In many instances, a polypeptide contains a polymerized amino acid residue sequence that is a transporter, an enzyme, a predicted protein of unknown function, or a domain or portion or fragment thereof. A transporter is involved in the movement of ions, small molecules, or macromolecules, such as a carbohydrate, across a biological membrane. An enzyme can catalyze a chemical reaction, such as the reduction of a carbohydrate to an alcohol, in a host cell. The polypeptide optionally contains modified amino acid residues, naturally occurring amino acid residues not encoded by a codon, and non-naturally occurring amino acid residues.

As used herein, “protein” refers to an amino acid sequence, oligopeptide, peptide, polypeptide, or portions thereof whether naturally occurring or synthetic.

Genes and proteins that may be used in the present disclosure include genes encoding conservatively modified variants and proteins that are conservatively modified variants of those genes and proteins described throughout the application. “Conservatively modified variants” as used herein include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Homologs of the genes and proteins described herein may also be used in the present disclosure. As used herein, “homology” refers to sequence similarity between a reference sequence and at least a fragment of a second sequence. Homologs may be identified by any method known in the art, preferably, by using the BLAST tool to compare a reference sequence to a single second sequence or fragment of a sequence or to a database of sequences. As described below, BLAST will compare sequences based upon percent identity and similarity. As used herein, “orthology” refers to genes in different species that derive from a common ancestor gene.

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 29% identity, optionally 30%, 40%, 45%, 50%, 55%, 60%, 65%, 70%, 75%, 80%, 85%, 90%, 95%, 99% or 100% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200, or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. When comparing two sequences for identity, it is not necessary that the sequences be contiguous, but any gap would carry with it a penalty that would reduce the overall percent identity. For blastn, the default parameters are Gap opening penalty=5 and Gap extension penalty=2. For blastp, the default parameters are Gap opening penalty=11 and Gap extension penalty=1.

A “comparison window,” as used herein, includes reference to a segment of any one of the number of contiguous positions including, but not limited to from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1981), by the homology alignment algorithm of Needleman and Wunsch (1970) J Mol Biol 48(3):443-453, by the search for similarity method of Pearson and Lipman (1988) Proc Natl Acad Sci USA 85(8):2444-2448, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection [see, e.g., Brent et al., (2003) Current Protocols in Molecular Biology, John Wiley & Sons, Inc. (Ringbou Ed)].

Two examples of algorithms that are suitable for determining percent sequence identity and sequence similarity are the BLAST and BLAST 2.0 algorithms, which are described in Altschul et al. (1997) Nucleic Acids Res 25(17):3389-3402 and Altschul et al. (1990) J. Mol. Biol 215(3)-403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4, and a comparison of both strands. For amino acid sequences, the BLASTP program uses as defaults a wordlength of 3, and expectation (E) of 10, and the BLOSUM62 scoring matrix [see Henikoff and Henikoff, (1992) Proc Natl Acad Sci USA 89(22):10915-10919] alignments (B) of 50, expectation (E) of 10, M=5, N=−4, and a comparison of both strands.

The BLAST algorithm also performs a statistical analysis of the similarity between two sequences (see, e.g., Karlin and Altschul, (1993) Proc Natl Acad Sci USA 90(12):5873-5877). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Other than percentage of sequence identity noted above, another indication that two nucleic acid sequences or polypeptides are substantially identical is that the polypeptide encoded by the first nucleic acid is immunologically cross-reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions. Yet another indication that two nucleic acid sequences are substantially identical is that the same primers can be used to amplify the sequence.

### Host Cells of the Present Disclosure

Certain aspects of the present disclosure relate to host cells containing one or more of a recombinant cellodextrin transporter, a recombinant cellodextrin phosphorylase, a recombinant β-glucosidase, a recombinant phosphoglucomutase, or a recombinant hexokinase. Such host cells may be used to degrade cellodextrin, to produce hydrocarbons or hydrocarbon derivatives from cellodextrin, or to reducing ATP consumption during glucose utilization.

“Host cell” and “host microorganism” are used interchangeably herein to refer to a living biological cell that can be transformed via insertion of recombinant DNA or RNA. Such recombinant DNA or RNA can be in an expression vector. Thus, a host organism or cell as described herein may be a prokaryotic organism (e.g., an organism of the kingdom Eubacteria) or a eukaryotic cell. As will be appreciated by one of ordinary skill in the art, a prokaryotic cell lacks a membrane-bound nucleus, while a eukaryotic cell has a membrane-bound nucleus.

Any prokaryotic or eukaryotic host cell may be used in the present disclosure so long as it remains viable after being transformed with a sequence of nucleic acids. Preferably, the host cell is not adversely affected by the transduction of the necessary nucleic acid sequences, the subsequent expression of the proteins or the resulting intermediates. Suitable eukaryotic cells include, without limitation, fungal, plant, insect and mammalian cells.

In preferred embodiments, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In certain embodiments, the fungal cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of the present disclosure, yeast shall be defined as described in Biology and Activities of Yeast (Skinner, F. A., Passmore, S. M., and Davenport, R. R., eds, Soc. App. Bacteriol. Symposium Series No. 9, 1980).

In some embodiments, the yeast host is a Candida, Hansenula, Issatchenkia, Kluyveromyces, Pichia, Saccharomyces, Schizosaccharomyces, or Yarrowia strain. In other embodiments, the yeast host is a Saccharomyces carlsbergensis (Todkar, 2010), Saccharomyces cerevisiae (Duarte et al., 2009), Saccharomyces diastaticus, Saccharomyces douglasii, Saccharomyces kluyveri, Saccharomyces norbensis, Saccharomyces monacensis (GB-Analysts Reports, 2008), Saccharomyces bayanus (Kristen Publicover, 2010), Saccharomyces pastorianus (Nakao et al., 2007), Saccharomyces pombe (Mousdale, 2008), or Saccharomyces oviformis strain. In yet other embodiments, the yeast host is Kluyveromyces lactis (O. W. Merten, 2001), Kluyveromyces fragilis (Pestal et al., 2006; Siso, 1996), Kluyveromyces marxiamus (K. Kourkoutas et al., 2008), Pichia stipitis (Almeida et al., 2008), Candida shehatae (Ayhan Demirbas, 2003), or Candida tropicalis (Jamai et al., 2006). In other embodiments, the yeast host may be Yarrowia lipolytica (Biryukova E. N., 2009), Brettanomyces custersii (Spindler D. D. et al., 1992), or Zygosaccharomyces roux (Chaabane et al., 2006). In one preferred embodiment, the yeast cell is S. cerevisiae.

In other embodiments, the fungal host is a filamentous fungal strain. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In some embodiments, the filamentous fungal host is, without limitation, an Acremonium, Aspergillus, Fusarium, Humicola, Mucor, Myceliophthora, Neurospora, Penicillium, Scytalidium, Thielavia, Tolypocladium, or Trichoderma strain.

In other embodiments, the filamentous fungal host is an Aspergillus awamori, Aspergillus foetidus, Aspergillus japonicus, Aspergillus nidulans, Aspergillus niger, or Aspergillus oryzae strain. In still other embodiments, the filamentous fungal host is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum strain. In yet other embodiments, the filamentous fungal host is a Humicola insolens, Humicola lanuginosa, Mucor miehei, Myceliophthora thermophila, Neurospora crassa, Penicillium purpurogenum, Scytalidium thermophilum, Sporotrichum thermophile (Topakas et al., 2003), or Thielavia terrestris strain. In a further embodiment, the filamentous fungal host is a Trichoderma harzianum, Trichoderma koningii, Trichoderma longibrachiatum, Trichoderma reesei, or Trichoderma viride strain.

In other embodiments, the host cell is prokaryotic, and in certain embodiments, the prokaryotes are E. coli (Dien, B. S. et al., 2003; Yomano, L. P. et al., 1998; Moniruzzaman et al., 1996), Bacillus subtilis (Susana Romero et al., 2007), Zymomonas mobilis (B. S. Dien et al., 2003; Weuster Botz, 1993; Alterthum and Ingram, 1989), Thermoanaerobacterium saccharolyticum (Marietta Smith, 2009), or Klebsiella oxytoca (Dien, B. S. et al., 2003; Zhou et al., 2001; Brooks and Ingram, 1995). In other embodiments, the prokaryotic host cells are Carboxydocella sp. (Dominik et al., 2007), Corynebacterium glutamicum (Masayuki Inui, et al., 2004), Enterobacteriaceae (Ingram et al., 1995), Erwinia chrysanthemi (Zhou and Ingram, 2000; Zhou et al., 2001), Lactobacillus sp. (McCaskey, T. A., et al., 1994), Pediococcus acidilactici (Zhou, S. et al., 2003), Rhodopseudomonas capsulata (X. Y. Shi et al., 2004), Streptococcus lactis (J. C. Tang et al., 1988), Vibrio furnissii (L. P. Wackett, 2010), Vibrio furnissii M1 (Park et al., 2001), Caldicellulosiruptor saccharolyticus (Z. Kadar et al., 2004), or Xanthomonas campestris (S. T. Yang et al., 1987). In other embodiments, the host cells are cyanobacteria. Additional examples of bacterial host cells include, without limitation, those species assigned to the Escherichia, Enterobacter, Azotobacter, Erwinia, Bacillus, Pseudomonas, Klebsiella, Proteus, Salmonella, Serratia, Shigella, Rhizobia, Vitreoscilla, Synechococcus, Synechocystis, and Paracoccus taxonomical classes.

The host cells of the present disclosure may be genetically modified in that recombinant nucleic acids have been introduced into the host cells, and as such the genetically modified host cells do not occur in nature. A suitable host cell of the present disclosure is one capable of expressing one or more nucleic acid constructs encoding one or more proteins for different functions.

“Recombinant nucleic acid” or “heterologous nucleic acid” or “recombinant polynucleotide” as used herein refers to a polymer of nucleic acids where at least one of the following is true: (a) the sequence of nucleic acids is foreign to (i.e., not naturally found in) a given host cell; (b) the sequence may be naturally found in a given host cell, but in an unnatural (e.g., greater than expected) amount; or (c) the sequence of nucleic acids contains two or more subsequences that are not found in the same relationship to each other in nature. For example, regarding instance (c), a recombinant nucleic acid sequence will have two or more sequences from unrelated genes arranged to make a new functional nucleic acid. Specifically, the present disclosure describes the introduction of an expression vector into a host cell, where the expression vector contains a nucleic acid sequence coding for a protein that is not normally found in a host cell or contains a nucleic acid coding for a protein that is normally found in a cell but is under the control of different regulatory sequences. With reference to the host cell's genome, then, the nucleic acid sequence that codes for the protein is recombinant. A protein that is referred to as recombinant generally implies that it is encoded by a recombinant nucleic acid sequence in the host cell.

A “recombinant” polypeptide, protein, or enzyme of the present disclosure, is a polypeptide, protein, or enzyme that is encoded by a “recombinant nucleic acid” or “heterologous nucleic acid” or “recombinant polynucleotide.”

In some embodiments, the genes encoding the desired proteins in the host cell may be heterologous to the host cell or these genes may be endogenous to the host cell but are operatively linked to heterologous promoters and/or control regions which result in the higher expression of the gene(s) in the host cell. In certain embodiments, the host cell does not naturally produce the desired proteins, and contains heterologous nucleic acid constructs capable of expressing one or more genes necessary for producing those molecules.

“Endogenous” as used herein with reference to a nucleic acid molecule or polypeptide and a particular cell or microorganism refers to a nucleic acid sequence or polypeptide that is in the cell and was not introduced into the cell using recombinant engineering techniques; for example, a gene that was present in the cell when the cell was originally isolated from nature.

“Genetically engineered” or “genetically modified” refers to any recombinant DNA or RNA method used to create a prokaryotic or eukaryotic host cell that expresses a protein at elevated levels, at lowered levels, or in a mutated form. In other words, the host cell has been transfected, transformed, or transduced with a recombinant polynucleotide molecule, and thereby been altered so as to cause the cell to alter expression of a desired protein. Methods and vectors for genetically engineering host cells are well known in the art; for example various techniques are illustrated in Current Protocols in Molecular Biology, Ausubel et al., eds. (Wiley & Sons, New York, 1988, and quarterly updates). Genetically engineering techniques include, without limitation, expression vectors, and targeted homologous recombination and gene activation (see, for example, U.S. Pat. No. 5,272,071).

Cellodextrin Transporters

Certain aspects of the present disclosure relate to host cells that contain a recombinant cellodextrin transporter. A cellodextrin transporter is any transmembrane protein that transports a cellodextrin molecule from outside of the cell to the inside of the cell and/or from inside of the cell to outside of the cell. In certain embodiments, the cellodextrin transporter is a functional fragment that maintains the ability to transport a cellodextrin molecule from outside of the cell to the inside of the cell and/or from inside of the cell to outside of the cell.

Recombinant cellodextrin transporters of the present disclosure may be encoded by any of the genes listed in Table 10, in Supplemental Data, Dataset S1, and page 3 in Tian et al., 2009; and in Tables 1 and 2.

TABLE 1
Listing of sequences encoding cellodextrin transporters.

| | Alter- | NCBI Reference | |
|---|---|---|---|
| Gene | nate | Sequence/GenBank | |
| Name/Locus | Name | Accession Number | Organism |
| NCU00801 | cbt1 | XP_963801.1/EAA34565 | N. crassa |
| NCU00809 | | XP_964302.1/EAA35116.1 | N. crassa |
| NCU00821 | AN25 | XP_964364.2/EAA35128.2 | N. crassa |
| NCU00988 | Xy33 | XP_963898.1/EAA34662.1 | N. crassa |
| NCU01231 | | XP_961597.2/EAA32361.2 | N. crassa |
| NCU01494 | AN49 | XP_955927.2/EAA26691.2 | N. crassa |
| NCU02188 | AN28-3 | XP_959582.2/EAA30346.2 | N. crassa |
| NCU04537 | Xy50 | XP_955977.1/EAA26741.1 | N. crassa |
| NCU04963 | AN29-2 | XP_959411.2/EAA30175.2 | N. crassa |
| NCU05519 | | XP_960481.1/EAA31245.1 | N. crassa |
| NCU05853 | | XP_959844.1/EAA30608.1 | N. crassa |
| NCU05897 | | XP_959888.1/EAA30652.1 | N. crassa |
| NCU06138 | Xy31 | XP_960000.1/EAA30764.1 | N. crassa |
| NCU08114 | cbt2 | XP_963873.1/EAA34637.1 | N. crassa |
| NCU09287 | AN41 | XP_958139.1/EAA28903.1 | N. crassa |
| NCU10021 | | XP_958069.2/EAA28833.2 | N. crassa |
| XP_001387242 | Ap26 | XP_001387242 | P. stipitis |
| HGT3 | Xyp30- | XP_001386715.1/ABN68686.1 | P. stipitis |
| | 1 | | |
| STL1 | Xyp30 | XP_001383774.1/ABN65745.1 | P. stipitis |
| STL12/XUT6 | Xyp29 | XP_001386589.1/ABN68560.1 | P. stipitis |
| SUT2 | Ap31 | XP_001384295.2/ABN66266.2 | P. stipitis |
| SUT3 | Xyp37 | XP_001386019.2/ABN67990.2 | P. stipitis |
| XUT1 | Xyp32 | XP_001385583.1/ABN67554.1 | P. stipitis |
| XUT2 | Xyp31 | XP_001387242.1/EAZ63219.2 | P. stipitis |
| XUT3 | Xyp33 | XP_001387138.1/EAZ63115.1 | P. stipitis |
| XUT7 | Xyp28 | XP_001387067.1/EAZ63044.1 | P. stipitis |
| NCU07705 | cdr-1 | XP_962291.1/EAA33055 | N. crassa |
| NCU05137 | | XP_956635.1/EAA27399 | N. crassa |
| NCU01517 | | XP_956966.1/EAA27730 | N. crassa |
| NCU09133 | | XP_958905.1/EAA29669 | N. crassa |
| NCU10040 | | | N. crassa |

TABLE 2
Listing of cellodextrin transporter orthologs.

| | | NCBI Reference |
|---|---|---|
| | | Sequence/NCBI |
| N. crassa | | GI Number/ |
| ortholog | Organism | JGI number ¥ |
| NCU00809 | Chaetomium globusom CBS148.51 | XP_001220480 |
| NCU00809 | Podospora anserina | XP_001912722 |
| NCU00809 | Nectria haematococca mpVI77-13-4 | EEU41662 |
| NCU00809 | Aspergillus nidulans FGSC A4 | XP_660803 |
| NCU00809 | Aspergillus terreus NIH2624 | XP_001218592 |
| NCU00809 | Talaromyces stipitatus ATCC 10500 | XP_002341594 |
| NCU00809 | Aspergillus niger | XP_001395979 |
| NCU00809 | Aspergillus fumigatus Af293 | XP_747891 |
| NCU00809 | Aspergillus terreus NIH2624 | XP_00120996 |
| NCU00809 | Aspergillus oryzae RIB40 | XP_001817400 |
| NCU08114 | Podospora anserina | XP_001908539 |
| NCU08114 | Penicillium chrysogenum Wisconsin | XP_002568019 |
| | 54-1255 | |
| NCU08114 | Aspergillus terreus NIH2624 | XP_001209810 |
| NCU08114 | Aspergillus oryzae RIB40 | XP_001820343 |
| NCU08114 | Aspergillus terreus NIH2624 | XP_001210859 |
| NCU08114 | Neurospora crassa OR74A | XP_001728155 |
| NCU08114 | Aspergillus oryzae RIB40 | XP_001826848 |
| NCU08114 | Aspergillus nidulans FGSC A4 | XP_657617 |
| NCU08114 | Talaromyces stipitatus ATCC 10500 | XP_002487579 |
| NCU08114 | Chaetomium globosum CBS 148.51 | XP_001227497 |
| NCU08114 | Trichoderma atroviridae | 215408 |
| NCU08114 | Chaetomium globosum | XP_001220290.1 |
| NCU08114 | Aspergillus nidulans | ANID_08347 |
| NCU08114 | Pleurotus ostreatus | 51322 |
| NCU08114 | Sporotrichum thermophile | 114107 |
| NCU00801 | Aspergillus nidulans | XP_660418.1 |
| NCU00801 | Magnaporthe grisea | XP_364883.1 |
| NCU00801 | Aspergillus fumigatus | XP_753099.1 |
| NCU00801 | Trichoderma atroviridae | 211304 |
| NCU00801 | Chaetomium globosum | XP_001220469.1 |
| NCU00801 | Tremella mesenterica | 63529 |
| NCU00801 | Heterobasidion. annosum | 105952 |
| NCU00801 | Cryphonectria parasitica | 252427 |
| NCU00801 | Trichoderma ressei | 67752 |
| NCU00801 | Aspergillus clavatus | XP_001268541.1 |
| NCU00801 | Neurospora discreta | 77429 |
| NCU00801 | Trichoderma reesei | 3405 |
| NCU00801 | Sporotrichum thermophile | 43941 |
| NCU00801 | Neurospora crassa | XP_963801.1 |
| NCU05853 | Chaetomium globosum | XP_001226269.1 |
| NCU05853 | Trichoderma reesei | 46819 |
| NCU05853 | Mycosphaerella graminicola | 68287 |
| NCU05853 | Aspergillus flavus | AFLA_000820A |
| NCU00809 | Pichia stipitis CBS6054 | XP_001383110.1/ |
| | | GI:126133170 |
| NCU00809 | Pichia stipitis CBS6054 | XP_001387231.1/ |
| | | GI:126276337 |
| NCU00809 | Pichia stipitis CBS6054 | XP_001383677.2/ |
| | | GI:150864727 |
| NCU08114 | Pichia stipitis CBS6054 | XP_001386873.1/ |
| | | GI:126275571 |
| NCU05853 | Pichia stipitis CBS6054 | XP_001382754.1/ |
| | | GI:126132458 |
| NCU08114 | Pichia stipitis CBS6054 | XP_001387757.1/ |
| | | GI:126273939 |
| NCU08114 | Pichia stipitis CBS6054 | XP_001385684.1/ |
| | | GI:126138322 |
| NCU08114 | Pichia stipitis CBS6054 | XP_001384653.2/ |
| | | GI:15086543 |

¥ When accession numbers were not available, the JGI number was used. The JGI number allows access to the gene sequence via the JGI genome portal for this organism (accessible from the following page: genome.jgi-psf.org/programs/fungi/index.jsf). The A. flavus and A. nidulans identifiers allow access to the genes through their genome portals at webpage cadre-genomes.org.uk/and webpage broadinstitute.org/annotation/genome/aspergillus_group/MultiHome.html, respectively.

In other embodiments, a recombinant cellodextrin transporter of the present disclosure has about 20%, or at least about 29%, or at least about 30%, or at least about 40%, or at least about 50%, or at least about 55%, or at least about 60%, or at least about 65%, or at least about 70%, or at least about 75%, or at least about 80%, or at least about 85%, or at least about 90%, or at least about 92%, or at least about 94%, or at least about 96%, or at least about 98%, or at least about 99%, or at least about 100% amino acid residue sequence identity to a polypeptide encoded by any of the genes listed in genes listed in Table 10, in Supplemental Data, Dataset S1, page 3 in Tian et al., 2009; and in Tables 1 and 2.

Additionally, cellodextrin transporters of the present disclosure include, without limitation, NCU00801, NCU00809, NCU08114, XP_{—}001268541.1, LAC2, NCU00130, NCU00821, NCU04963, NCU07705, NCU05137, NCU01517, NCU09133, and NCU10040. In certain embodiments, the recombinant cellodextrin transporter has at least about 20%, or at least about 29%, or at least about 30%, or at least about 40%, or at least about 50%, or at least about 55%, or at least about 60%, or at least about 65%, or at least about 70%, or at least about 75%, or at least about 80%, or at least about 85%, or at least about 90%, or at least about 92%, or at least about 94%, or at least about 96%, or at least about 98%, or at least about 99%, or at least about 100% amino acid residue sequence identity to a polypeptide encoded by any of the sequences NCU00801, NCU00809, NCU08114, XP_{—}001268541.1, LAC2, NCU00130, NCU00821, NCU04963, NCU07705, NCU05137, NCU01517, NCU09133, or NCU10040.

In certain preferred embodiments, the host cell contains a cellodextrin transporter encoded by NCU00801, which is also known as CDT-1 or CBT1. In other preferred embodiments, the host cell contains a cellodextrin transporter encoded by NCU08114, which is also known as CDT-2 or CBT2. In certain some embodiments, the recombinant cellodextrin transporter has an amino acid sequence with at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to CDT-1 (SEQ ID NO: 9) or CDT-2 (SEQ ID: 10).

Suitable cellodextrin transporters of the present disclosure also include, without limitation, those described in U.S. Pat. Application Publication No. US 2011/0262983 and PCT Publication No. WO 2011/123715. For example, suitable cellodextrin transporters may include, without limitation, HXT2.1, HXT2.2, HXT2.3, HXT2.4, HXT2.5, HXT2.6, and HXT4. In certain embodiments, a recombinant cellodextrin transporter of the present disclosure has about 20%, or at least about 29%, or at least about 30%, or at least about 40%, or at least about 50%, or at least about 55%, or at least about 60%, or at least about 65%, or at least about 70%, or at least about 75%, or at least about 80%, or at least about 85%, or at least about 90%, or at least about 92%, or at least about 94%, or at least about 96%, or at least about 98%, or at least about 99%, or at least about 100% amino acid residue sequence identity to amino acid residue sequence identity to a polypeptide encoded by any of the genes listed in genes listed in U.S. Pat. Application Publication No. US 2011/0262983 and PCT Publication No. WO 2011/123715 (e.g., HXT2.1, HXT2.2, HXT2.3, HXT2.4, HXT2.5, HXT2.6, or HXT4).

Recombinant cellodextrin transporters of the present disclosure may also include, without limitation, polypeptides encoded by polynucleotides that encode conservatively modified variants of polypeptides encoded by the genes listed above. Recombinant cellodextrin transporters of the present disclosure further include polypeptides encoded by polynucleotides that encode homologs or orthologs of polypeptides encoded by any of the genes listed in Table 10, in Supplemental Data, Dataset S1, page 3 in Tian et al., 2009; in Tables 1 and 2, and in U.S. Pat. Application Publication No. US 2011/0262983 and PCT Publication No. WO 2011/123715.

Cellodextrin Transporter Sequence Motifs

As described herein, recombinant cellodextrin transporters of the present disclosure include members of the Major Facilitator Superfamily sugar transporter family, including, without limitation, NCU00988, NCU10021, NCU04963, NCU06138, NCU00801, NCU08114, and NCU05853. Members of the Major Facilitator Superfamily (MFS) (Transporter Classification #2.A. 1) of transporters almost always contain 12 transmembrane α-helices, with an intracellular N- and C-terminus (Pao et al., Microbiol Mol Biol Rev 62, 1, March 1998). While the primary sequence of MFS transporters varies widely, all are thought to share the tertiary structure of the E. coli lactose permease (LacY) (J. Abramson et al., Science 301, 610, 2003), and the E. coli Pi/glycerol-3-phospate (GlpT) (Huang et al., Science 301, 616, 2003). In these examples the six N- and C-terminal helices form two distinct domains connected by a long cytoplasmic loop between helices 6 and 7. This symmetry corresponds to a duplication event thought to have given rise to the MFS. Substrate binds within a hydrophilic cavity formed by helices 1, 2, 4, and 5 of the N-terminal domain, and helices 7, 8, 10, and 11 of the C-terminal domain. This cavity is stabilized by helices 3, 6, 9, and 12.

The Sugar Transporter family of the MFS (Transporter Classification #2.A.1.1) is defined by motifs found in transmembrane helices 6 and 12 (PESPR (SEQ ID NO: 231)/PETK (SEQ ID NO: 232)), and loops 2 and 8 (GRR/GRK) (M. C. Maiden, E. O. Davis, S. A. Baldwin, D. C. Moore, P. J. Henderson, Nature 325, 641 (Feb. 12-18, 1987)). The entire Hidden Markov Model (HMM) for this family can be viewed at pfam.janelia.org/family/PF00083#tabview=tab3. PROSITE (N. Hulo et al., Nucleic Acids Res 34, D227 (Jan. 1, 2006)) uses two motifs to identify members of this family. The first is [LIVMSTAG]-[LIVMFSAG]-{SH}-{RDE}-[LIVMSA]-[DE]-{TD}-[LIVMFYWA]-G-R-[RK]-x(4,6)-[GSTA] (SEQ ID NO: 198). The second is [LIVMF]-x-G-[LIVMFA]-{V}-x-G-{KP}-x(7)-[LIFY]-x(2)-[EQ]-x(6)-[RK] (SEQ ID NO: 199). As an example of how to read a PROSITE motif, the following motif, [AC]-x-V-x(4)-{ED}(SEQ ID NO: 200), is translated as: [Ala or Cys]-any-Val-any-any-any-any-{any but Glu or Asp}(SEQ ID NO: 200).

Multiple sequence alignments produced in T-COFFEE between putative cellodextrin transporter orthologs and confirmed cellodextrin transporters identified conserved motifs. The conserved motifs were defined using PROSITE notation. As an example of how to read a PROSITE motif, the following motif, [AC]-x-V-x(4)-{ED}(SEQ ID NO: 200), is translated as: [Ala or Cys]-any-Val-any-any-any-any-{any but Glu or Asp}(SEQ ID NO: 200). Transmembrane helix 1 contains the motif, [LIVM]-Y-[FL]-x(13)-[YF]-D (SEQ ID NO: 1). Transmembrane helix 2 contains the motif, [YF]-x(2)-G-x(5)-[PVF]-x(6)-[DQ] (SEQ ID NO: 2). The loop connecting transmembrane helix 2 and transmembrane helix 3 contains the motif, G-R-[RK] (SEQ ID NO: 3). Transmembrane helix 5 contains the motif, R-x(6)-[YF]-N (SEQ ID NO: 4). Transmembrane helix 6 contains the motif, WR-[IVLA]-P-x(3)-Q (SEQ ID NO: 5). The sequence between transmembrane helix 6 and transmembrane helix 7 contains the motif, P-E-S-P-R-x-L-x(8)-A-x(3)-L-x(2)-Y-H (SEQ ID NO: 6). Transmembrane helix 7 contains the motif, F-[GST]-Q-x-S-G-N-x-[LIV] (SEQ ID NO: 7). Transmembrane helix 10 and transmembrane helix 11 and the sequence between them contains the motif, L-x(3)-[YIV]-x(2)-E-x-L-x(4)-R-[GA]-K-G (SEQ ID NO: 8).

Accordingly, certain aspects of the present disclosure relate to recombinant cellodextrin transporters, or functional fragments thereof, that contain one or more of the disclosed conserved motifs. In certain embodiments, the recombinant cellodextrin transporter, or functional fragment thereof, includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 1 contains SEQ ID NO: 1. In other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 2 contains SEQ ID NO: 2. In still other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and a loop connecting transmembrane α-helix 2 and transmembrane α-helix 3 contains SEQ ID NO: 3. In yet other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 5 contains SEQ ID NO: 4. In other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 6 contains SEQ ID NO: 5. In still other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and sequence between transmembrane α-helix 6 and transmembrane α-helix 7 contains SEQ ID NO: 6. In yet other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 7 contains SEQ ID NO: 7. In other embodiments, the recombinant cellodextrin transporter includes a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, and transmembrane α-helix 10 and transmembrane α-helix 11 and the sequence between them contain SEQ ID NO: 8.

Moreover, each of the above described embodiments may be combined in any number of combinations. A recombinant cellodextrin transporter according to any of the above embodiments may include a polypeptide containing 1, 2, 3, 4, 5, 6, or 7 of any of SEQ ID NOs: 1-8, or the polypeptide may contain all of SEQ ID NOs: 1-8. For example, a recombinant cellodextrin transporter may include a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, where transmembrane α-helix 1 contains SEQ ID NO: 1, a loop connecting transmembrane α-helix 2 and transmembrane α-helix 3 contains SEQ ID NO: 3, and transmembrane α-helix 7 contains SEQ ID NO: 7. Or, in another example, a recombinant cellodextrin transporter may include a polypeptide containing transmembrane α-helix 1, α-helix 2, α-helix 3, α-helix 4, α-helix 5, α-helix 6, α-helix 7, α-helix 8, α-helix 9, α-helix 10, α-helix 11, α-helix 12, where transmembrane α-helix 2 contains SEQ ID NO: 2, transmembrane α-helix 3 contains SEQ ID NO: 3, transmembrane α-helix 6 contains SEQ ID NO: 5, and transmembrane α-helix 10 and transmembrane α-helix 11 and the sequence between them contain SEQ ID NO: 8.

Mutant Cellodextrin Transporters

Other aspects of the present disclosure relate to mutant cellodextrin transporters that may be used to increase the function and/or activity of a cellodextrin transporter of the present disclosure. Mutant cellodextrin transporters may be produced by mutating a polynucleotide encoding a cellodextrin transporter of the present disclosure. In some embodiments, a mutant cellodextrin transporter of the present disclosure may contain at least one mutation that includes, without limitation, a point mutation, a missense mutation, a substitution mutation, a frameshift mutation, an insertion mutation, a duplication mutation, an amplification mutation, a translocation mutation, or an inversion mutation that results in a cellodextrin transporter with increased function and/or activity.

Methods of generating at least one mutation in a cellodextrin transporter of interest are well known in the art and include, without limitation, random mutagenesis and screening, site-directed mutagenesis, PCR mutagenesis, insertional mutagenesis, chemical mutagenesis, and irradiation.

In some embodiments, the mutant cellodextrin transporter contains one or more amino acid substitutions. For example, a cellodextrin transporter of the present disclosure may contain an amino acid substitution at one or more positions corresponding to positions of the amino acid sequence of CDT-1 (SEQ ID NO: 9). Suitable one or more positions include, without limitation, a position corresponding to amino acid 91 of SEQ ID NO: 9, a position corresponding to amino acid 104 of SEQ ID NO: 9, a position corresponding to amino acid 170 of SEQ ID NO: 9, a position corresponding to amino acid 174 of SEQ ID NO: 9, a position corresponding to amino acid 194 of SEQ ID NO: 9, a position corresponding to amino acid 213 of SEQ ID NO: 9, a position corresponding to amino acid 335 of SEQ ID NO: 9, and combinations thereof.

In one non-limiting example, the amino acid substitution at one or more positions are a glycine (G) to alanine (A) substitution at a position corresponding to amino acid 91 of SEQ ID NO: 9; a glutamine (Q) to alanine (A) substitution at a position corresponding to amino acid 104 of SEQ ID NO: 9; a phenylalanie (F) to alanine (A) substitution at a position corresponding to amino acid 170 of SEQ ID NO: 9; an arginine (R) to alanine (A) substitution at a position corresponding to amino acid 174 of SEQ ID NO: 9; a glutamate (E) to alanine (A) substitution at a position corresponding to amino acid 194 of SEQ ID NO: 9; a phenylalanie (F) to lysine (L) substitution at a position corresponding to amino acid 213 of SEQ ID NO: 9; a phenylalanie (F) to alanine (A) substitution at a position corresponding to amino acid 335 of SEQ ID NO: 9; or combinations thereof. In certain preferred embodiments, the amino acid substitution at one or more positions is a glycine (G) to alanine (A) substitution at a position corresponding to amino acid 91 of SEQ ID NO: 9 and/or a phenylalanie (F) to lysine (L) substitution at a position corresponding to amino acid 213 of SEQ ID NO: 9.

In some embodiments, the increased function and/or activity of a mutant cellodextrin transporter results in a host cell that consumes cellodextrin at a rate faster than the rate of cellodextrin consumption in a cell lacking the mutant cellodextrin transporter. For example, the rate of cellodextrin consumption in a host cell containing a mutant cellodextrin transporter may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 225%, at least 250%, at least 275%, at least 300%, or at least a higher percentage faster than the rate of cellodextrin consumption in a host cell containing a corresponding wild-type cellodextrin transporter.

Combinations with Cellodextrin Transporters

Further aspects of the present disclosure relate to host cells that contain at least one recombinant cellodextrin transporter of the present disclosure in combination with one or more of a recombinant cellodextrin phosphorylase of the present disclosure, a recombinant r-glucosidase of the present disclosure, a recombinant phosphoglucomutase of the present disclosure, and a recombinant hexokinase of the present disclosure.

Generally, once a cellodextrin transporter of the present disclosure transports cellodextrins, such as cellobiose, into the cell, the cell must degrade the cellodextrins. However, in certain embodiments, host cells of the present disclosure, such as yeast cells, do not naturally contain the enzymes necessary to degrade the cellodextrins into less complex saccharides that can be utilized by the cell. Accordingly, the host cell can be engineered to express a recombinant cellodextrin phosphorylase and/or a recombinant β-glucosidase in order to degrade the cellodextrins. Thus, in certain embodiments, a host cell of the present disclosure expresses at least one recombinant cellodextrin transporter in combination with at least one recombinant cellodextrin phosphorylase and/or at least one recombinant β-glucosidase. Moreover, under different stress conditions, it can be beneficial to express both a recombinant cellodextrin phosphorylase and a recombinant β-glucosidase in cells containing a cellodextrin transporter. Additionally, such host cells can be engineered to optimize the cellodextrin phosphorylase pathway and β-glucosidase pathway under different stress conditions. Accordingly, in certain embodiments, a host cell of the present disclosure containing a cellodextrin transporter also expresses at least one recombinant cellodextrin phosphorylase and at least one recombinant β-glucosidase.

In some embodiments, host cells containing a cellodextrin transporter are capable of phosphorolytically cleaving cellodextrin to glucose-1-phosphate and a shorter-chain cellodextrin. In order for the cell to utilize the produced glucose-1-phosphate, it needs to be converted to glucose-6-phosphate in order to enter the cell's glycolytic pathway. Glucose-1-phosphate can be converted to glucose-6-phosphate by phosphoglucomutases that are naturally expressed in the host cell. However, phosphoglucomutases can be transcriptionally downregulated during glycolytic growth. Moreover, the expression of other recombinant proteins or enzymes, or the growth conditions utilized may also affect the expression of endogenous phosphoglucomutases in the cell. These shortcomings may be overcome by recombinantly expressing at least one phosphoglucomutase in the cell. Accordingly, in certain embodiments, a host cell of the present disclosure expresses at least one recombinant cellodextrin transporter in combination with at least one recombinant phosphoglucomutase.

In other embodiments host cells containing a cellodextrin transporter are capable of degrading cellodextrins, such as cellobiose, to glucose. However, in order for the cell to utilize glucose, the glucose needs to be phosphorylated to glucose-6-phosphate. Generally, glucose is phosphorylated to glucose-6-phosphate by hexokinases that are naturally expressed in the cell. However, host cells of the present disclosure may not express a sufficient amount of hexokinase activity to efficiently convert all the glucose produced by the degradation of cellodextrins. Moreover, the expression of other recombinant proteins or enzymes, or the growth conditions utilized may also affect the expression of endogenous hexokinases in the cell. These shortcomings may be overcome by recombinantly expressing at least one hexokinase in the cell. Accordingly, in certain embodiments, a host cell of the present disclosure expresses at least one recombinant cellodextrin transporter in combination with at least one recombinant hexokinase.

Cellodextrin Phosphorylases

Other aspects of the present disclosure relate to host cells that contain a recombinant cellodextrin phosphorylase. Cellodextrin phosphorylases of the present disclosure catalyze the degradation of a cellodextrin by utilizing inorganic phosphate to cleave β-glucosidic linkages between glucose moieties in the cellodextrin. Cellodextrin phosphorylases of the present disclosure may include polypeptides having EC 2.4.1.49 activity, which catalyze the following reaction: (1,4-β-D-glucosyl)ₙ+inorganic phosphate⇄(1,4-β-D-glucosyl)ₙ₋₁+α-D-glucose-1-phosphate. Polypeptides having EC 2.4.1.49 activity belong to the GH 94 family of glycoside hydrolases. Polypeptides with EC 2.4.1.49 activity include, without limitation, 1,4-beta-D-oligo-D-glucan:phosphate alpha-D-glucosyltransferases and beta-1,4-oligoglucan:orthophosphate glucosyltransferases.

Cellodextrin phosphorylases of the present disclosure also include cellobiose phosphorylase enzymes having EC 2.4.1.20 activity, which catalyze the following reaction: cellobiose+inorganic phosphate⇄α-D-glucose-1-phosphate+D-glucose. Enzymes having EC 2.4.1.20 activity belong to the hexosyltransferase family of glycoside hydrolases. Enzymes with EC 2.4.1.20 activity include, without limitation, cellobiose phosphorylases and cellobiose:phosphate alpha-D-glucosyltransferases.

In certain embodiments, a cellodextrin phosphorylase of the present disclosure is a functional fragment that maintains the catalytic activity of the corresponding full length cellodextrin phosphorylase.

Suitable cellodextrin phosphorylases may be obtained from cellulolytic microorganisms. Examples of such microorganisms include, without limitation, Celvibrio gilvus, Sacharophagus degradans, and Clostridium thermocellum. Examples of suitable cellodextrin phosphorylases include, without limitation, those listed in Table 3, homologs thereof, and orthologs thereof.

Table 3: Cellobiose Phosphorylases

TABLE 3
Cellobiose Phosphorylases

| NCBI Reference | |
|---|---|
| Sequence | Organism |
| YP_001036707.1 | Clostridium_thermocellum_ATCC_27405 |
| ZP_06248015.1 | Clostridium thermocellum JW20 |
| BAA25846.1 | Clostridium thermocellum |
| ZP_07328762.1 | Acetivibrio cellulolyticus CD2 |
| YP_004462192.1 | Mahella australiensis 50-1 BON |
| ZP_08194540.1 | Clostridium papyrosolvens DSM 2782 |
| YP_002506434.1 | Clostridium cellulolyticum H10 |
| YP_002534325.1 | Thermotoga neapolitana DSM 4359 |
| NP_229644.1 | Thermotoga maritime MSB8 |
| YP_001244545.1 | Thermotoga petrophila RKU-1 |
| AAB95491.2 | Thermotoga neapolitana |
| CAB16926.1 | Thermotoga neapolitana |
| YP_003839734. | Caldicellulosiruptor obsidiansis OB47 |
| YP_004797912.1 | Caldicellulosiruptor lactoaceticus 6A |
| YP_004027280.1 | Caldicellulosiruptor kristjanssonii 177R1B |
| YP_004001699.1 | Caldicellulosiruptor owensensis OL |
| YP_002572365.1 | Caldicellulosiruptor bescii DSM 6725 |
| YP_001557556.1 | Clostridium phytofermentans ISDg |
| YP_004024831.1 | Caldicellulosiruptor kronotskyensis 2002 |
| YP_003841585.1 | Clostridium cellulovorans 743B |
| YP_003993282.1 | Caldicellulosiruptor hydrothermalis 108 |
| YP_001179895.1 | Caldicellulosiruptor saccharolyticus DSM |
| | 8903 |
| AAC45510.1 | Clostridium stercorarium |
| ZP_02421512.1 | Eubacterium siraeum DSM 15702 |
| CBL33370.1 | Eubacterium siraeum V10Sc8a |
| YP_004309477.1 | Clostridium lentocellum DSM 5427 |
| CBK97022.1 | Eubacterium siraeum 70/3 |
| YP_526792.1 | Saccharophagus degradans 2-40 |
| YP_002352548.1 | Dictyoglomus turgidum DSM 6724 |
| YP_002250367.1 | Dictyoglomus thermophilum H-6-12 |
| ZP_08848217.1 | Anaerophaga thermohalophila DSM 12881 |
| YP_003870668.1 | Paenibacillus polymyxa E681 |
| YP_003861246.1 | Maribacter sp. HTCC2170 |
| YP_003074163.1 | Teredinibacter turnerae T7901 |
| AEJ60757.1 | Spirochaeta thermophila DSM 6578 |
| CBL18325.1 | Ruminococcus sp. 18P13 |
| ZP_08470173.1 | Dysgonomonas mossii DSM 22836 |
| YP_003875014.1 | Spirochaeta thermophila DSM 6192 |
| ZP_01113063.1 | Reinekea sp. MED297 |
| ZP_06145290.1 | Ruminococcus flavefaciens FD-1 |
| YP_004042937.1 | Paludibacter propionicigenes WB4 |
| ZP_08158670.1 | Ruminococcus albus 8 |
| YP_004103378.1 | Ruminococcus albus 7 |
| YP_002509717.1 | Halothermothrix orenii H 168 |
| CBK83462.1 | Coprococcus sp. ART55/1 |
| ZP_02074262.1 | Clostridium sp. L2-50 |
| ZP_02206204.1 | Coprococcus eutactus ATCC 27759 |
| CBL11091.1 | Roseburia intestinalis XB6B4 |
| CBL09808.1 | Roseburia intestinalis M50/1 |
| ZP_06201842.1 | Bacteroides sp. D20 |
| ZP_02068984.1 | Bacteroides uniformis ATCC 8492 |
| ADD61402.1 | Uncultured organism |
| YP_002936968.1 | Eubacterium rectale ATCC 33656 |
| CBK93948.1 | Eubacterium rectale M104/1 |
| ZP_07882835.1 | Prevotella buccae ATCC 33574 |
| ZP_06418755.1 | Prevotella buccae D17 |
| YP_004837718.1 | Roseburia hominis A2-183 |
| ZP_06252207.1 | Prevotella copri DSM 18205 |
| ZP_03754075.1 | Roseburia inulinivorans DSM 16841 |
| CBK73469.1 | Butyrivibrio fibrisolvens 16/4 |
| ZP_07059510.1 | Prevotella bryantii B14 |
| ZP_07838874.1 | Eubacterium cellulosolvens 6 |
| YP_003573551.1 | Prevotella ruminicola 23 |
| YP_004451849.1 | Cellulomonas fimi ATCC 484 |
| gi\|109157379\|pdb\|2CQS | Cellvibrio gilvus |
| gi\|315364402\|pdb\|3ACT\| | Cellvibrio gilvus |
| gi\|315364400\|pdb\|3ACS | Cellvibrio gilvus |

In some embodiments, the cellodextrin phosphorylase is a cellobiose phosphorylase (CBP). Examples of suitable cellobiose phosphorylases include, without limitation, the Celvibrio gilvus cellobiose phosphorylase CgCBP, the Sacharophagus degradans cellobiose phosphorylase SdCBP, the Clostridium thermocellum cellobiose phosphorylase CtCBP, homologs thereof, and orthologs thereof.

TABLE 4
Cellobiose Phosphorylases

| NCBI Reference | |
|---|---|
| Sequence | Organism |
| C0FUS5 | Roseburia inulinivorans |
| D1PD04 | Prevotella copri |
| A8S7N6 | Faecalibacterium prausnitzii |
| B0MKI1 | Eubacterium siraeum |
| G0VV46 | Paenibacillus polymyxa |
| Q8VP44 | Clostridium thermocellum |
| C7H9T5 | Faecalibacterium prausnitzii |
| D4KUQ1 | Roseburia intestinalis |
| D3HVI4 | Prevotella buccae |
| E4K6D0 | Caldicellulosiruptor lactoaceticus |
| A7A4F8 | Bifidobacterium adolescentis |
| F7KEC8 | Lachnospiraceae bacterium |
| C7GDZ2 | Roseburia intestinalis |
| D8DTL6 | Prevotella bryantii |
| O66383 | Clostridium thermocellum |
| E1KJE9 | Acetivibrio cellulolyticus |
| C6LE12 | Marvinbryantia formatexigens |
| O87964 | Thermotoga neapolitana |
| E5V814 | Bacteroides sp. |
| A7VDP1 | Clostridium sp. |
| A7UYL3 | Bacteroides uniformis |
| E9SB92 | Ruminococcus albus |
| Q59316 | Clostridium stercorarium |
| D5HEX9 | Coprococcus sp. |
| F9D678 | Prevotella dentalis |
| A4BA76 | Reinekea blandensis |
| D4LUD5 | Ruminococcus obeum |
| C7HGL0 | Clostridium thermocellum |
| A4Q9G7 | Cellulomonas flavigena |
| A8SQ49 | Coprococcus eutactus |
| D4KSF6 | Roseburia intestinalis |
| A5ZX10 | Ruminococcus obeum |
| D4JK01 | Eubacterium rectale |
| D6KW25 | Scardovia inopinata |
| Q7WTR6 | Cellulomonas uda |
| D4JVA3 | Eubacterium siraeum |
| C6IVN7 | Paenibacillus sp. |
| D4MHY7 | Eubacterium siraeum |
| F1THV9 | Clostridium papyrosolvens |
| E2ZJ10 | Faecalibacterium sp. |
| D4LFD0 | Ruminococcus sp. |
| O66264 | Cellvibrio gilvus |
| E6K807 | Prevotella buccae |
| D4J030 | Butyrivibrio fibrisolvens |
| E4MK94 | Eubacterium cellulosolvens |
| D2EVQ9 | Bacteroides sp. |
| O52504 | Thermotoga neapolitana |
| D6DYH9 | Eubacterium rectale |
| F8X006 | Dysgonomonas mossii |
| D1NLD9 | Clostridium thermocellum |
| D4K816 | Faecalibacterium prausnitzii |
| C4G2K5 | Abiotrophia defectiva |
| A5IL96 | Thermotoga petrophila |
| E4T5F8 | Paludibacter propionicigenes |
| E0RKP2 | Paenibacillus polymyxa |
| E4S6A9 | Caldicellulosiruptor kristjanssonii |
| E3EI03 | Paenibacillus polymyxa |
| A4XIG9 | Caldicellulosiruptor saccharolyticus |
| B8DZK1 | Dictyoglomus turgidum |
| B8I421 | Clostridium cellulolyticum |
| F8A760 | Cellvibrio gilvus |
| B5YCV9 | Dictyoglomus thermophilum |
| E4SGY7 | Caldicellulosiruptor kronotskyensis |
| Q9X2G3 | Thermotoga maritima |
| E6ULX2 | Clostridium thermocellum |
| C5BMK9 | Teredinibacter turnerae |
| B9K7M6 | Thermotoga neapolitana |
| Q21L49 | Saccharophagus degradans |
| D5UG84 | Cellulomonas flavigena |
| E0RZR4 | Butyrivibrio proteoclasticus |
| B9MNR3 | Anaerocellum thermophilum |
| D1BX62 | Xylanimonas cellulosilytica |
| D5EVL5 | Prevotella ruminicola |
| C4Z6T4 | Eubacterium eligens |
| A9KHF0 | Clostridium phytofermentans |
| D2C6W3 | Thermotoga naphthophila |
| E4Q363 | Caldicellulosiruptor owensensis |
| A4AVU9 | Maribacter sp. |
| E4Q7Z5 | Caldicellulosiruptor hydrothermalis |
| F4H6V3 | Cellulomonas fimi |
| D9SMN6 | Clostridium cellulovorans |
| D9TIB6 | Caldicellulosiruptor obsidiansis |
| B8CZK3 | Halothermothrix orenii |
| F2JIB3 | Cellulosilyticum lentocellum |
| E6UCF2 | Ruminococcus albus |
| C4ZGN7 | Eubacterium rectale |
| D9R0E3 | Clostridium saccharolyticum |
| F3ZVZ6 | Mahella australiensis |
| A3DC35 | Clostridium thermocellum |
| B1LAH0 | Thermotoga sp. |
| D9ZDJ8 | Uncultured organism |

A cellodextrin phosphorylase of the present disclosure may include, without limitation, the Clostridium lentocellum cellodextrin phosphorylase CDP_Clent, the Clostridium thermocellum cellodextrin phosphorylase CDP_Ctherm, and the Acidovibrio cellulolyticus cellodextrin phosphorylase CDP_Acell. In certain embodiments, the cellodextrin phosphorylase has an amino acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to that of CDP_Clent, CDP_Ctherm, or CDP_Acell.

In certain preferred embodiments, a cellodextrin phosphorylase of the present disclosure has an amino acid sequence with at least about 20%, at least about 25%, at least about 30%, at least about 40%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 100% amino acid identity to CgCBP (SEQ ID NO: 11), SdCBP (SEQ ID NO: 12), or CtCBP (SEQ ID NO: 13).

In other embodiments, a host cell of the present disclosure contains at least one additional recombinant cellodextrin phosphorylase, or functional fragment thereof. Preferably, the at least one additional recombinant cellodextrin phosphorylase is a cellobiose phosphorylase selected from CgCBP, SdCBP, and CtCBP.

Cellodextrin Phosphorylase Sequence Motifs

The amino acid sequences of Clostridium thermocellum (BAA22081.1), Acidovibrio cellulolyticus (ZP_{—}07328763.1) and Clostridium lentocellum (YP_{—}004310865.1) cellodextrin phosphorylases were simultaneously analyzed by PSI-BLAST to identify polypeptides that are annotated as “cellodextrin phosphorylases.” All such identified polypeptides were then used as inputs for a second round of PSI-BLAST. From these results, the cellodextrin phosphorylase sequences were analyzed by PRATT analysis (ExPASy Bioinformatics website), which identified a conserved PROSITE motif. The conserved sequence is: G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 14). As an example of how to read a PROSITE motif, the following motif, [AC]-x-V-x(4)-{ED}(SEQ ID NO: 200), is translated as: [Ala or Cys]-any-Val-any-any-any-any-{any but Glu or Asp}(SEQ ID NO: 200). This conserved motif may be used to identify further cellodextrin phosphorylases. For example, SEQ ID NO: 14 was used to identify 16 additional cellodextrin phosphorylases by using the PROSITE server (PROSITE ExPASy website). Accordingly, suitable cellodextrin phosphorylases of the present disclosure include the 16 identified cellodextrin phosphorylases listed in Table 4.

TABLE 4
Cellodextrin Phosphorylases Identified by PROSITE

| NCBI Reference | |
|---|---|
| Sequence | Organism |
| F9AJY7 | Vibrio cholerae |
| G0GFK3 | Spirochaeta thermophila |
| Q93HT8 | Clostridium thermocellum |
| P77846 | Clostridium stercorarium |
| O24780 | Clostridium thermocellum |
| C7HEH9 | Clostridium thermocellum |
| F9SHD3 | Vibrio splendidus |
| A3UQH5 | Vibrio splendidus |
| A5KVQ6 | Vibrionales bacterium |
| A3XS61 | Vibrio sp. |
| D1NRB6 | Clostridium thermocellum |
| C9RP13 | Fibrobacter succinogenes |
| E0RQX7 | Spirochaeta thermophila |
| A3DJQ6 | Clostridium thermocellum |
| E6UTK1 | Clostridium thermocellum |
| B7VTD2 | Vibrio splendidus |

Amino acid sequence alignment and PRATT analysis (ExPASy Bioinformatics website) of the cellobiose phosphorylase proteins listed in Table 3 revealed that these proteins contain a conserved PROSITE motif. The conserved sequence is: Y-Q-[CN]-M-[IV]-T-F-[CN]-[HLMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 15). This conserved motif may be used to identify other cellobiose phosphorylases. For example, SEQ ID NO: 15 was used to identify 91 additional cellobiose phosphorylases by using the PROSITE server (PROSITE ExPASy website). Accordingly, suitable cellobiose phosphorylases of the present disclosure include the 91 identified cellobiose phosphorylases listed in Table 5. Specific cellodextrin phosphorylases may be preferred depending on the cellodextrin transporter that may be contained in the host cell.

TABLE 5
Cellobiose Phosphorylases Identified by PROSITE

| NCBI Reference | |
|---|---|
| Sequence | Organism |
| C0FUS5 | Roseburia inulinivorans |
| D1PD04 | Prevotella copri |
| A8S7N6 | Faecalibacterium prausnitzii |
| B0MKI1 | Eubacterium siraeum |
| G0VV46 | Paenibacillus polymyxa |
| Q8VP44 | Clostridium thermocellum |
| C7H9T5 | Faecalibacterium prausnitzii |
| D4KUQ1 | Roseburia intestinalis |
| D3HVI4 | Prevotella buccae |
| E4K6D0 | Caldicellulosiruptor lactoaceticus |
| A7A4F8 | Bifidobacterium adolescentis |
| F7KEC8 | Lachnospiraceae bacterium |
| C7GDZ2 | Roseburia intestinalis |
| D8DTL6 | Prevotella bryantii |
| O66383 | Clostridium thermocellum |
| E1KJE9 | Acetivibrio cellulolyticus |
| C6LE12 | Marvinbryantia formatexigens |
| O87964 | Thermotoga neapolitana |
| E5V814 | Bacteroides sp. |
| A7VDP1 | Clostridium sp. |
| A7UYL3 | Bacteroides uniformis |
| E9SB92 | Ruminococcus albus |
| Q59316 | Clostridium stercorarium |
| D5HEX9 | Coprococcus sp. |
| F9D678 | Prevotella dentalis |
| A4BA76 | Reinekea blandensis |
| D4LUD5 | Ruminococcus obeum |
| C7HGL0 | Clostridium thermocellum |
| A4Q9G7 | Cellulomonas flavigena |
| A8SQ49 | Coprococcus eutactus |
| D4KSF6 | Roseburia intestinalis |
| A5ZX10 | Ruminococcus obeum |
| D4JK01 | Eubacterium rectale |
| D6KW25 | Scardovia inopinata |
| Q7WTR6 | Cellulomonas uda |
| D4JVA3 | Eubacterium siraeum |
| C6IVN7 | Paenibacillus sp. |
| D4MHY7 | Eubacterium siraeum |
| F1THV9 | Clostridium papyrosolvens |
| E2ZJ10 | Faecalibacterium sp. |
| D4LFD0 | Ruminococcus sp. |
| O66264 | Cellvibrio gilvus |
| E6K807 | Prevotella buccae |
| D4J030 | Butyrivibrio fibrisolvens |
| E4MK94 | Eubacterium cellulosolvens |
| D2EVQ9 | Bacteroides sp. |
| O52504 | Thermotoga neapolitana |
| D6DYH9 | Eubacterium rectale |
| F8X006 | Dysgonomonas mossii |
| D1NLD9 | Clostridium thermocellum |
| D4K816 | Faecalibacterium prausnitzii |
| C4G2K5 | Abiotrophia defectiva |
| A5IL96 | Thermotoga petrophila |
| E4T5F8 | Paludibacter propionicigenes |
| E0RKP2 | Paenibacillus polymyxa |
| E4S6A9 | Caldicellulosiruptor kristjanssonii |
| E3EI03 | Paenibacillus polymyxa |
| A4XIG9 | Caldicellulosiruptor saccharolyticus |
| B8DZK1 | Dictyoglomus turgidum |
| B8I421 | Clostridium cellulolyticum |
| F8A760 | Cellvibrio gilvus |
| B5YCV9 | Dictyoglomus thermophilum |
| E4SGY7 | Caldicellulosiruptor kronotskyensis |
| Q9X2G3 | Thermotoga maritima |
| E6ULX2 | Clostridium thermocellum |
| C5BMK9 | Teredinibacter turnerae |
| B9K7M6 | Thermotoga neapolitana |
| Q21L49 | Saccharophagus degradans |
| D5UG84 | Cellulomonas flavigena |
| E0RZR4 | Butyrivibrio proteoclasticus |
| B9MNR3 | Anaerocellum thermophilum |
| D1BX62 | Xylanimonas cellulosilytica |
| D5EVL5 | Prevotella ruminicola |
| C4Z6T4 | Eubacterium eligens |
| A9KHF0 | Clostridium phytofermentans |
| D2C6W3 | Thermotoga naphthophila |
| E4Q363 | Caldicellulosiruptor owensensis |
| A4AVU9 | Maribacter sp. |
| E4Q7Z5 | Caldicellulosiruptor hydrothermalis |
| F4H6V3 | Cellulomonas fimi |
| D9SMN6 | Clostridium cellulovorans |
| D9TIB6 | Caldicellulosiruptor obsidiansis |
| B8CZK3 | Halothermothrix orenii |
| F2JIB3 | Cellulosilyticum lentocellum |
| E6UCF2 | Ruminococcus albus |
| C4ZGN7 | Eubacterium rectale |
| D9R0E3 | Clostridium saccharolyticum |
| F3ZVZ6 | Mahella australiensis |
| A3DC35 | Clostridium thermocellum |
| B1LAH0 | Thermotoga sp. |
| D9ZDJ8 | Uncultured organism |

Additionally, the x-ray crystal structure of the Cellvibrio gilvus cellobiose phosphorylase was used with PDB ID 3QG0, and was analyzed by PRATT analysis (ExPASy Bioinformatics website) to identify a conserved PROSITE motif that is conserved among both cellobiose phosphorylases and cellodextrin phosphorylases. The conserved motif is: Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233). This conserved motif may be used to identify further cellobiose phosphorylases and cellodextrin phosphorylases.

Accordingly, certain aspects of the present disclosure relate to cellodextrin phosphorylases and cellobiose phosphorylases having a conserved motif. In certain embodiments, a cellodextrin phosphorylase or cellobiose phosphorylase of the present disclosure, or functional fragment thereof, contains the sequence SEQ ID NO: 233. In other embodiments, a cellodextrin phosphorylase of the present disclosure, or functional fragment thereof, contains the sequence SEQ ID NO: 14. In further embodiments, a cellobiose phosphorylase of the present disclosure, or functional fragment thereof, contains the sequence SEQ ID NO: 15.

Mutant Cellodextrin Phosphorylases

Other aspects of the present disclosure relate to mutant cellodextrin phosphorylases that may be used to increase the function and/or activity of a cellodextrin phosphorylase of the present disclosure. In certain embodiments, the mutant cellodextrin phosphorylase is a cellobiose phosphorylase. Mutant cellodextrin phosphorylases may be produced by mutating a polynucleotide encoding a cellodextrin phosphorylase of the present disclosure. In some embodiments, a mutant cellodextrin phosphorylase of the present disclosure may contain at least one mutation that includes, without limitation, a point mutation, a missense mutation, a substitution mutation, a frameshift mutation, an insertion mutation, a duplication mutation, an amplification mutation, a translocation mutation, or an inversion mutation that results in a cellodextrin phosphorylase with increased function and/or activity.

Methods of generating at least one mutation in a cellodextrin phosphorylase of interest are well known in the art and include, without limitation, random mutagenesis and screening, site-directed mutagenesis, PCR mutagenesis, insertional mutagenesis, chemical mutagenesis, and irradiation.

In some embodiments, the mutant cellodextrin phosphorylase contains one or more amino acid substitutions. For example, a cellodextrin phosphorylase of the present disclosure may contain an amino acid substitution at one or more positions corresponding to positions of the amino acid sequence of any of the cellodextrin phosphorylases or cellobiose phosphorylases listed in Tables 3 and 4, respectively. Additionally, a cellodextrin phosphorylase of the present disclosure may contain an amino acid substitution at one or more positions corresponding to positions of the amino acid sequence of CDP_Clent, CDP_Ctherm, or CDP_Acell. In other embodiments, a cellobiose phosphorylase of the present disclosure may contain an amino acid substitution at one or more positions corresponding to positions of the amino acid sequence of CgCBP (SEQ ID NO: 11), SdCBP (SEQ ID NO: 12), or CtCBP (SEQ ID NO: 13).

Additionally, a mutant cellobiose phosphorylase of the present disclosure may contain an amino acid substitution at one or more positions corresponding to positions of the amino acid sequence of SdCBP (SEQ ID NO: 12). Suitable one or more positions include, without limitation, a position corresponding to amino acid 409 of SEQ ID NO: 12, a position corresponding to amino acid 482 of SEQ ID NO: 12, a position corresponding to amino acid 484 of SEQ ID NO: 12, a position corresponding to amino acid 651 of SEQ ID NO: 12, a position corresponding to amino acid 653 of SEQ ID NO: 12, and combinations thereof.

In one non-limiting example, the amino acid substitution at one or more positions are an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; or combinations thereof. In certain preferred embodiments, the amino acid substitution at one or more positions is an isoleucine (I) to methionine (M) substitution at a position corresponding to amino acid 409 of SEQ ID NO: 12 and/or an asparagine (N) to aspartate (D) substitution at a position corresponding to amino acid 482 of SEQ ID NO: 12.

In some embodiments, the increased function and/or activity of a mutant cellodextrin phosphorylase results in a host cell that degrades cellodextrins, such as cellobiose, at a greater rate than the rate of cellodextrin degradation in a cell expressing a wild-type (i.e., non-mutant) cellodextrin phosphorylase. For example, the rate of cellodextrin degradation in a host cell containing a mutant cellodextrin phosphorylase may be at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%, at least 125%, at least 150%, at least 175%, at least 200%, at least 225%, at least 250%, at least 275%, at least 300%, or at least a higher percentage greater than the rate of cellodextrin degradation in a host cell containing a corresponding wild-type cellodextrin phosphorylase.

Combinations with Cellodextrin Phosphorylases

Further aspects of the present disclosure relate to host cells that contain at least one recombinant cellodextrin phosphorylase of the present disclosure in combination with one or more of a recombinant β-glucosidase of the present disclosure, a recombinant phosphoglucomutase of the present disclosure, and a recombinant hexokinase of the present disclosure.

In some embodiments, host cells of the present disclosure containing a recombinant cellodextrin phosphorylase are capable of transporting cellodextrins, such as cellobiose, into the cell. Such host cells can transport cellodextrins by expressing endogenous proteins or recombinant proteins that transport cellodextrins, such as cellobiose, into the cell. In such embodiments, host cells containing a recombinant cellodextrin phosphorylase can be grown under different stress conditions where it can be beneficial to express the recombinant cellodextrin phosphorylase in combination with a recombinant β-glucosidase. Additionally, the host cells can be engineered to optimize the cellodextrin phosphorylase pathway and β-glucosidase pathway under different stress conditions. Accordingly, in certain embodiments, a host cell of the present disclosure expresses at least one recombinant cellodextrin phosphorylase in combination with at least one recombinant β-glucosidase.

In other embodiments, host cells containing a recombinant cellodextrin phosphorylase phosphorolytically cleave cellodextrins to glucose-1-phosphate and shorter-chain cellodextrins. In such embodiments, the glucose-1-phosphate needs to be converted to glucose-6-phosphate in order to be utilized by the cell. Glucose-1-phosphate can be converted to glucose-6-phosphate by phosphoglucomutases that are naturally expressed in the host cell. However, phosphoglucomutases can be transcriptionally downregulated during glycolytic growth. Moreover, the expression of other recombinant proteins or enzymes, or the growth conditions utilized may also affect the expression of endogenous phosphoglucomutases in the cell. These shortcomings may be overcome by recombinantly expressing at least one phosphoglucomutase in the cell. Accordingly, in certain embodiments, a host cell of the present disclosure expresses at least one recombinant cellodextrin phosphorylase in combination with at least one recombinant phosphoglucomutase.

In further embodiments, host cells containing a recombinant cellodextrin phosphorylase are capable of degrading cellodextrins, such as cellobiose, to glucose. In order for the cell to utilize the produced glucose, the glucose needs to be phosphorylated to glucose-6-phosphate. Generally, glucose is phosphorylated to glucose-6-phosphate by hexokinases that are naturally expressed in the cell. However, host cells of the present disclosure may not express a sufficient amount of hexokinase activity to efficiently phosphorylate all the produced glucose. Moreover, the expression of other recombinant proteins or enzymes, or the growth conditions utilized may also affect the expression of endogenous hexokinases in the cell. These shortcomings may be overcome by recombinantly expressing at least one hexokinase in the cell. Accordingly, in certain embodiments, a host cell of the present disclosure expresses at least one recombinant cellodextrin phosphorylase in combination with at least one recombinant hexokinase.

β-Glucosidases

Further aspects of the present disclosure relate to host cells that utilize an intracellular β-glucosidase in addition to a cellodextrin phosphorylase, or in embodiments where ATP is not limiting, in place of a cellodextrin phosphorylase. The β-glucosidase may be endogenous or recombinant to the host cell. As used herein, β-glucosidase refers to a β-D-glucoside glucohydrolase (E.C. 3.2.1.21), which catalyzes the hydrolysis of terminal non-reducing β-D-glucose residues with the release of β-D-glucose. A β-glucosidase is any enzyme that catalyzes the hydrolysis of terminal non-reducing residues in β-D-glucosides with release of glucose.

In certain embodiments, a β-glucosidase of the present disclosure is a functional fragment that maintains the catalytic activity of the corresponding full length β-glucosidase.

Suitable β-glucosidases include, without limitation, members of the Glycosyl Hydrolase Family 1 (GH1) family of glycosyl hydrolases. In some embodiments, the β-glucosidase is from N. crassa, and in certain preferred embodiments, the β-glucosidase is NCU00130, which is also known as GH1-1. Suitable β-glucosidases of the present disclosure also include homologs and orthologs of NCU00130. Examples of NCU00130 include, without limitation, T. melanosporum, CAZ82985.1; A. oryzae, BAE57671.1; P. placenta, EED81359.1; P. chrysosporium, BAE87009.1; Kluyveromyces lactis, CAG99696.1; Laccaria bicolor, EDR09330; Clavispora lusitaniae, EEQ37997.1; and Pichia stipitis, ABN67130.1.

β-Glucosidase Sequence Motifs

As disclosed herein, β-glucosidases of the present disclosure include members of the GH1 family of glycosyl hydrolases. PRATT analysis (ExPASy Bioinformatics website) of members of this group identified the presence of two conserved PROSITE motifs. The first PROSITE motif matches a conserved portion of the N-terminus and has the sequence: F-x-[FYWM]-[GSTA]-x-[GSTA]-x-[GSTA](2)-[FYNH]-[NQ]-x-E-x-[GSTA] (SEQ ID NO: 16). The second PROSITE motif matches a conserved portion of the surrounding active site and has the sequence: [LIVMFSTC]-[LIVFYS]-[LIV]-[LIVMST]-E-N-G-[LIVMFAR]-[CSAGN] (SEQ ID NO: 17). Here, E is the catalytic glutamate. As an example of how to read a PROSITE motif, the following motif, [AC]-x-V-x(4)-{ED}(SEQ ID NO: 200), is translated as: [Ala or Cys]-any-Val-any-any-any-any-{any but Glu or Asp}(SEQ ID NO: 200). While these two conserved motifs may be generally used to identify further β-glucosidases, it should be noted that not all β-glucosidases of the GH1 family of glycosyl hydrolases will contain both conserved motifs. For example, NCU00130 contains the conserved motif of SEQ ID NO: 16 but lacks the conserved motif of SEQ ID NO: 17.

Additional suitable β-glucosidases include those from the Glycosyl Hydrolase Family 3 family of glycosyl hydrolases. PRATT analysis (ExPASy Bioinformatics website) of members of this group identified the presence of a PROSITE motif that matched a conserved portion of the surrounding active site. The conserved sequence is [LIVM](2)-[KR]-x-[EQKRD]-x(4)-G-[LIVMFTC]-[LIVT]-[LIVMF]-[ST]-D-x(2)-[SGADNIT] (SEQ ID NO: 18). Here D is the catalytic aspartate. This conserved motif may be used to identify further β-glucosidases.

Moreover, suitable β-glucosidases may also include any β-glucosidase that contains the conserved domain of β-glucosidase/6-phospho-β-glucosidase/β-galactosidase found in NCBI sequence COG2723. Specific β-glucosidases may be preferred depending on the cellodextrin transporter that may be contained in the host cell.

Accordingly, certain aspects of the present disclosure relate to β-glucosidases having one or more conserved motifs. In certain embodiments, a β-glucosidase of the present disclosure, or functional fragment thereof, contains one or more sequences selected from SEQ ID NO: 16, SEQ ID NO: 17, and SEQ ID NO: 18. In other embodiments, a β-glucosidase of the present disclosure, or functional fragment thereof, contains two or more sequences selected from SEQ ID NO: 16, SEQ ID NO: 17, and SEQ ID NO: 18.

Combinations with β-Glucosidases

Further aspects of the present disclosure relate to host cells that contain at least one recombinant β-glucosidase of the present disclosure in combination with one or more of a recombinant phosphoglucomutase of the present disclosure and a recombinant hexokinase of the present disclosure.

In some embodiments, host cells of the present disclosure containing a recombinant β-glucosidase are capable of transporting cellodextrins, such as cellobiose, into the cell. Such host cells can transport cellodextrins by expressing endogenous proteins or recombinant proteins that transport cellodextrins, such as cellobiose, into the cell.

In some embodiments, host cells containing a recombinant β-glucosidase are also capable of phosphorolytically cleaving cellodextrins. The host cell may phosphorolytically cleave cellodextrins by either expressing an endogenous or a recombinant cellodextrin phosphorylase. Alternatively, the host cell may express an alternative pathway that results in the phosphorolytic cleavage of cellodextrins. In such embodiments, the phosphorolytic cleavage of cellodextrins results in the production of glucose-1-phosphate. However, in order for the cell to utilize the produced glucose-1-phosphate, it must convert the glucose-1-phosphate to glucose-6-phosphate. Glucose-1-phosphate can be converted to glucose-6-phosphate by phosphoglucomutases that are naturally expressed in the host cell. However, phosphoglucomutases can be transcriptionally downregulated during glycolytic growth. Moreover, the expression of other recombinant proteins or enzymes, or the growth conditions utilized may also affect the expression of endogenous phosphoglucomutases in the cell. These shortcomings may be overcome by recombinantly expressing at least one phosphoglucomutase in the cell. Accordingly, in certain embodiments, a host cell of the present disclosure expresses at least one recombinant β-glucosidase in combination with at least one recombinant phosphoglucomutase.

In other embodiments, host cells containing a recombinant β-glucosidase are capable of degrading cellodextrins, such as cellobiose, to glucose. In order for the cell to utilize the produced glucose, the glucose needs to be phosphorylated to glucose-6-phosphate. Generally, glucose is phosphorylated to glucose-6-phosphate by hexokinases that are naturally expressed in the cell. However, host cells of the present disclosure may not express a sufficient amount of hexokinase activity to efficiently phosphorylate all the produced glucose. Moreover, the expression of other recombinant proteins or enzymes, or the growth conditions utilized may also affect the expression of endogenous hexokinases in the cell. These shortcomings may be overcome by recombinantly expressing at least one hexokinase in the cell. Accordingly, in certain embodiments, a host cell of the present disclosure expresses at least one recombinant β-glucosidase in combination with at least one recombinant hexokinases.

Phosphoglucomutases

Other aspects of the present disclosure relate to host cells that contain a phosphoglucomutase. As used herein, a “phosphoglucomutase” refers to a polypeptide having EC 5.4.2.2 activity, which catalyzes the transfer of a phosphate group on an α-D-glucose monomer from the 1′ position to the 6′ position in the forward direction or the 6′ position to the 1′ position in the reverse direction. In particular, a polypeptide having EC 5.4.2.2 activity catalyzes the interconversion of glucose-1-phosphate and glucose-6-phosphate. In certain embodiments, a phosphoglucomutase of the present disclosure is a functional fragment that maintains the catalytic activity of the corresponding full length phosphoglucomutase.

Phosphoglucomutases of the present disclosure may be expressed either endogenously or ectopically in a host cell of the present disclosure. In embodiments where a phosphoglucomutase is expressed ectopically in a host cell of the present disclosure, the host cell further contains a recombinant phosphoglucomutase.

In certain preferred embodiments, the phosphoglucomutase is the S. cerevisiae phosphoglucomutase PGM2. In other embodiments, the phosphoglucomutase is a homolog or ortholog of the S. cerevisiae phosphoglucomutase PGM2. In further embodiments, the phosphoglucomutase is overexpressed.

Phosphoglucomutase Sequence Motifs

Amino acid sequence alignment and PRATT analysis (ExPASy Bioinformatics website) of known phosphoglucomutase genes revealed that these proteins contain a conserved PROSITE motif. The conserved sequence is: [GSA]-[LIVMF]-x-[LIVM]-[ST]-[PGA]-S-H-[NIC]-P (SEQ ID NO: 19). This conserved motif may be used to identify further phosphoglucomutases.

Accordingly, certain aspects of the present disclosure relate to phosphoglucomutases, or functional fragments thereof, having a conserved motif. In certain embodiments, a phosphoglucomutase of the present disclosure contains the sequence SEQ ID NO: 19.

Combinations with Phosphoglucomutase

Further aspects of the present disclosure relate to host cells that contain at least one recombinant phosphoglucomutase in combination with one or more recombinant hexokinases of the present disclosure.

In some embodiments, host cells of the present disclosure containing a recombinant phosphoglucomutase are capable of transporting cellodextrins, such as cellobiose, into the cell and are capable of degrading the transported cellodextrin. Such host cells can transport cellodextrins by expressing endogenous proteins or recombinant proteins that transport cellodextrins, such as cellobiose, into the cell. These host cells can also degrade the transported cellodextrins by expressing endogenous proteins or recombinant proteins that degrade cellodextrins. In such embodiments, host cells containing a recombinant phosphoglucomutase are capable of degrading cellodextrins, such as cellobiose, to glucose. In order for the cell to utilize the produced glucose, the glucose needs to be phosphorylated to glucose-6-phosphate. Generally, glucose is phosphorylated to glucose-6-phosphate by hexokinases that are naturally expressed in the cell. However, host cells of the present disclosure may not express a sufficient amount of hexokinase activity to efficiently phosphorylate all the produced glucose. Moreover, the expression of other recombinant proteins or enzymes, or the growth conditions utilized may also affect the expression of endogenous hexokinases in the cell. These shortcomings may be overcome by recombinantly expressing at least one hexokinase in the cell. Accordingly, in certain embodiments, a host cell of the present disclosure expresses at least one recombinant phosphoglucomutase in combination with at least one recombinant hexokinase.

Hexokinases

Further aspects of the present disclosure relate to host cells that contain a hexokinase. As used herein, a “hexokinase” refers to a polypeptide having EC 2.7.1.1 activity, which catalyzes the phosphorylation of a six-carbon sugar, a hexose, to a hexose phosphate. Preferably, hexokinases of the present disclosure phosphorylate glucose. In certain embodiments, a hexokinase of the present disclosure is a functional fragment that maintains the catalytic activity of the corresponding full length hexokinase.

Hexokinases of the present disclosure may be expressed either endogenously or ectopically in a host cell of the present disclosure. In embodiments where a hexokinase is expressed ectopically in a host cell of the present disclosure, the host cell further contains a recombinant hexokinase.

In certain embodiments, the hexokinase is the S. cerevisiae hexokinase HXK1, HXK2, or GLK1. Preferably, the hexokinase is the S. cerevisiae hexokinase HXK1. In other embodiments, the hexokinase is a homolog or ortholog of the S. cerevisiae hexokinase HXK1, HXK2, or GLK1. In further embodiments, the hexokinase is overexpressed.

Hexokinase Sequence Motifs

Amino acid sequence alignment and PRATT analysis (ExPASy Bioinformatics website) of known hexokinase genes revealed that these proteins contain a conserved PROSITE motif. The conserved sequence is: [LIVM]-G-F-[TN]-F-S-[FY]-P-x(5)-[LIVM]-[DNST]-x(3)-[LIVM]-x(2)-W-T-K-x-[LF] (SEQ ID NO: 20). This conserved motif may be used to identify further hexokinases.

Accordingly, certain aspects of the present disclosure relate to hexokinases, or functional fragments thereof, having a conserved motif. In certain embodiments, a hexokinase of the present disclosure contains the sequence SEQ ID NO: 20.

Glucose Response Genes

Host cells of the present disclosure may further contain one or more glucose response genes. The one or more glucose response genes may be recombinant or endogenous to the host cell. As used herein, a “glucose response gene” refers to any gene encoding a protein that is involved in a cell responding to glucose. Typically, the proteins encoded by glucose response genes allow a cell to “sense” or “perceive” the amount of glucose available to the cell for nutrients and to set metabolic and growth rates to match the available amount of glucose. The activities of the proteins encoded by glucose response genes ensure that the metabolism of the cell is optimal and glucose is efficiently utilized.

In preferred embodiments, the one or more glucose response genes are selected from Snf3, Rgt1, Rgt2, Yck1/2, Std1, Mthy1, Snf1/4, Grr1, Gpr1, Gpa2, Ras2, Stb3, Hxk2, Pfk27, Pfk26, Sch9, Yak1, Mig1, Rim15, Kcs1, and Tps1. In other embodiments, the one or more glucose response genes may be orthologs of Snf3, Rgt1, Rgt2, Yck1/2, Std1, Mthy1, Snf1/4, Grr1, Gpr1, Gpa2, Ras2, Stb3, Hxk2, Pfk27, Pfk26, Sch9, Yak1, Mig1, Rim15, Kcs1, or Tps1; or any genes encoding polypeptides having at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the polypeptides encoded by Snf3, Rgt1, Rgt2, Yck1/2, Std1, Mthy1, Snf1/4, Grr1, Gpr1, Gpa2, Ras2, Stb3, Hxk2, Pfk27, Pfk26, Sch9, Yak1, Mig1, Rim15, Kcs1, or Tps1.

Snf3 (NM_{—}001180254.1) encodes a high affinity glucose sensor that detects low concentrations of extracellular glucose (Ozcan, PNAS, 1996).

Rgt1 (NM_{—}001179604.1) encodes a glucose-responsive transcription factor that regulates expression of several glucose transporter (HXT) genes in response to glucose (Kim and Johnston, J Biol Chem, 2006).

Rgt2 (NM_{—}001180198.1) is a low affinity glucose sensor that detects high concentrations of extracellular glucose (Ozcan, PNAS, 1996).

Yck1/2 (NM_{—}001179265.1, NM_{—}001182992.1) is a membrane-associated casein kinase I involved in glucose sensing. It is activated by the transmembrane portion of Snf3 or Rgt2. Yck1/2 phosphorylates Mthy1 and Std1 bound to Snf3 or Rgt2, which triggers their recognition by Grr1 ubiquitin ligase and subsequent degradation (Moriya and Johnston, PNAS, 2004).

The absence of Std1 (NM_{—}001183466.1) or Mthy1 (NM_{—}001180585.1) leads to phosphorylation of Rgt1, which prevents its binding to DNA and repression of HXT genes.

Snf1/4 (NM_{—}001180785.1, NM_{—}001180980.1) together form the SNF1 kinase complex, which is inactivated upon addition of glucose.

Grr1 (NM_{—}001181747.1) is a ubiquitin ligase containing Mthy1, Std1, Pfk27, Tye7, Stp2, Aro1, His4, Hom3, and Mae1.

Gpr1 (NM_{—}001180094.1) is a G-protein coupled receptor in the cAMP-PKA pathway. When sugar phosphates are present, it induces cAMP production in response to glucose (Rolland et al., FEMS Yeast Res, 2002).

Gpa2 (NM_{—}001178911.1) is a G-alpha subunit associated with Gpr1 in the cAMP-PKA pathway. When sugar phosphates are present, it induces cAMP production in response to glucose (Rolland et al., FEMS Yeast Res, 2002).

Ras2 (NM_{—}001182936.1) is a GTP binding protein that appears to play a role in the transcriptional changes that take place in the cell in response to glucose (Wang et al., PLOS Biology, 2004).

Stb3 (NM_{—}001180476.1) is a ribosomal RNA processing element (RRPE)-binding protein that represses transcription of growth genes. This repression is relieved by glucose (Liko et al., Genetics, 2010).

Hxk2 (NM_{—}001181119.1) is the predominant isoenzyme used during growth on glucose, fructose, and mannose. It is repressed by non-fermentable carbon sources. Hxk2 is necessary for a full transcriptional response to glucose and regulates its own expression. Hxk2-mediated transcriptional responses do not correlate to hexokinase activity of Hxk2, suggesting that the signaling and kinase activity of Hxk2 are distinct (Moreno and Herrero, FEMS Microbiol Rev, 2002).

Pfk27 (NM_{—}001183390.1) catalyzes the synthesis of fructose-2,6-bisphosphate, a second messenger that activates glycolysis and inhibits gluconeogenesis (Benanti, Nat Cell Biol, 2007). It is induced and stabilized by fermentable carbon sources. Pfk27 is phosphorylated by Snf1 and targeted for degradation by Grr1.

Pfk26 (NM_{—}001179455.1) catalyzes the synthesis of fructose-2,6-bisphosphate, a second messenger that activates glycolysis and inhibits gluconeogenesis.

Sch9 (NM_{—}0011799336.1) is a protein kinase involved in transcriptional activation of osmostress-responsive genes; regulates G1 progression, cAPK activity, nitrogen activation of the FGM pathway; involved in life span regulation; homologous to mammalian Akt/PKB.

Yak1 (NM_{—}001181574.1) is a serine-threonine protein kinase that is part of a glucose-sensing system involved in growth control in response to glucose availability; translocates from the cytoplasm to the nucleus and phosphorylates Pop2p in response to a glucose signal.

Mig1 (NM_{—}00180900.1) is a transcription factor involved in glucose repression; sequence specific DNA binding protein containing two Cys2His2 zinc finger motifs; regulated by the SNF1 kinase and the GLC7 phosphatase.

Rim15 (NM_{—}001179933.1) is a glucose-repressible protein kinase involved in signal transduction during cell proliferation in response to nutrients, specifically the establishment of stationary phase; identified as a regulator of IME2; substrate of Pho80p-Pho85p kinase.

Kcs1 (NM_{—}001180325.1) is an inositol hexakisphosphate (IP6) and inositol heptakisphosphate (IP7) kinase. Generation of high energy inositol pyrophosphates by Kcs1p is required for many processes such as vacuolar biogenesis, stress response and telomere maintenance.

Tps1 (NM_{—}00117874.1) is a synthase subunit of trehalose-6-phosphate synthase/phosphatase complex, which synthesizes the storage carbohydrate trehalose; also found in a monomeric form; expression is induced by the stress response and repressed by the Ras-cAMP pathway.

Altered Protein Activity Level

In host cells containing one or more glucose response genes, the activity level of one or more proteins encoded by the one or more glucose response genes may be altered compared to the wild-type activity level of the one or more proteins. The activity level of the one or more proteins may be increased or decreased compared to the wild-type activity level of the one or more protein.

Alterations in activity level of proteins can be achieved by genetic modifications of the host cell. Genetic modifications that result in an increase in gene expression or protein activity can be referred to as amplification, overproduction, overexpression, activation, enhancement, addition, or up-regulation of a gene. It may include increased expression and/or activity of the encoded proteins and includes higher activity or action of the proteins (e.g., specific activity or in vivo enzymatic activity), reduced inhibition or degradation of the proteins, and overexpression of the proteins. For example, gene copy number can be increased, expression levels can be increased by use of a promoter that gives higher levels of expression than that of the native promoter, or a gene can be altered by genetic engineering or classical mutagenesis to increase the biological activity of an enzyme or action of a protein. Mutations that cause a gene to be continuously expressed or that cause the encoded protein to be constitutively active are additional examples of genetic modifications that result in an increase in protein activity. Combinations of any of the modifications described above are also possible.

Genetic modifications that result in a decrease in gene expression or protein activity can be referred to as inactivation (complete or partial), deletion, interruption, blockage, silencing, down-regulation, or attenuation of expression of a gene. For example, a genetic modification in a gene which results in a decrease in the activity of the protein encoded by such gene, can be the result of a complete deletion of the gene (i.e., the gene does not exist, and therefore the protein does not exist), a mutation in the gene which results in incomplete or no translation of the protein (e.g., the protein is not expressed), or a mutation in the gene which decreases or abolishes the natural function of the protein (e.g., a protein is expressed which has decreased or no enzymatic activity or action). More specifically, reference to decreasing the action of proteins discussed herein generally refers to any genetic modification in the host cell in question, which results in decreased expression and/or biological activity of the proteins and includes decreased activity of the proteins, increased inhibition or degradation of the proteins, and reduction or elimination of expression of the proteins. For example, the action or activity of a protein can be decreased by blocking or reducing the production of the protein, reducing protein action, or inhibiting the action of the protein. Combinations of some of these modifications are also possible. Blocking or reducing the production of a protein can include placing the gene encoding the protein under the control of a promoter that requires the presence of an inducing compound in the growth medium. By establishing conditions such that the inducer becomes depleted from the medium, the expression of the gene encoding the protein (and therefore, of protein synthesis) could be turned off. Blocking or reducing the action of a protein could also include using an excision technology approach similar to that described in U.S. Pat. No. 4,743,546. To use this approach, the gene encoding the protein of interest is cloned between specific genetic sequences that allow specific, controlled excision of the gene from the genome. Excision could be prompted by, for example, a shift in the cultivation temperature of the culture, as in U.S. Pat. No. 4,743,546, or by some other physical or nutritional signal.

In general, according to the present disclosure, an alteration in the activity of a protein is made with reference to the same characteristic of a wild-type (i.e., normal, not modified) protein that is derived from the same organism (from the same source or parent sequence), which is measured or established under the same or equivalent conditions. Such conditions include the assay or culture conditions (e.g., medium components, temperature, pH, etc.) under which the activity of the protein is measured, as well as the type of assay used, the host cell that is evaluated, etc. As discussed above, equivalent conditions are conditions (e.g., culture conditions) which are similar, but not necessarily identical (e.g., some conservative changes in conditions can be tolerated), and which do not substantially change the effect on cell growth or biological activity as compared to a comparison made under the same conditions.

Pentose Transporters

Host cells of the present disclosure may further contain at least one recombinant pentose transporter, which allows the cells to utilize hemicellulosic pentose sugars for the production of hydrocarbons or hydrocarbon derivatives. A pentose transporter is any transmembrane protein that transports a pentose molecule from outside of the cell to the inside of the cell and/or from inside of the cell to outside of the cell. Pentose, as used herein, refers to any monosaccharide with five carbon atoms. Examples of pentoses include, without limitation, xylose, arabinose, mannose, galactose, and rhamnose.

In certain embodiments, a pentose transporter of the present disclosure is a functional fragment that maintains the ability to transport a pentose molecule from outside of the cell to the inside of the cell and/or from inside of the cell to outside of the cell.

Examples of suitable pentose transporters include, without limitation, those listed in Table 6.

TABLE 6
Pentose Transporters

| Gene Name | Organism | NCBI Reference Sequence |
|---|---|---|
| Ap31/SUT2 | P. stipitis | ABN66266 |
| Ap26/XP_001387242 | P. stipitis | XP 001387242 |
| AN49/NCU01494 | N. crassa | EAA2669I |
| AN41/NCU09287 | N. crassa | EAA28903 |
| AN29-2/NCU04963 | N. crassa | EAA30175 |
| AN28-3/NCU02188 | N. crassa | EAA30346 |
| AN25/NCU00821 | N. crassa | EAA35128 |
| Xy50/NCU04537 | N. crassa | EAA26741 |
| Xy31/NCU06138 | N. crassa | EAA30764 |
| Xy33/NCU00988 | N. crassa | EAA34662 |
| Xyp37/SUT3 | P. stipitis | ABN67990 |
| Xyp33/XUT3 | P. stipitis | EAZ63115 |
| Xyp32/XUTl | P. stipitis | ABN67554 |
| Xyp30/STLl | P. stipitis | ABN65745 |
| Xyp31/XUT2 | P. stipitis | AAVQOIOOOO02 |
| Xyp29/STLl2/XUT6 | P. stipitis | ABN68560 |
| Xyp30-1/HGT3 | P. stipitis | ABN68686 |
| Xyp28/XUT7 | P. stipitis | EAZ63044 |

In certain embodiments, the pentose transporter is a xylose transporter. Examples of suitable xylose transporters include, without limitation, those listed in Table 7.

TABLE 7
Xylose Transporters

| NCBI Reference | |
|---|---|
| Sequence | Organism |
| XP_002488227 | Talaromyces stipitatus |
| XP_001400900 | Aspergillus niger |
| XP_001220481 | Chaetomium globosum CBS 48.51 |
| XP_001912725 | Podospora anserina |
| XP_660079 | Aspergillus nidulans FGSC A4 |
| AAL89823 | Aspergillus niger |
| XP_002382573 | Aspergillus flavus NRRL3357 |
| XP_459386 | Debaryomyces hansenii CBS767 |
| XP_001825132 | Aspergillus oryzae RIB40 |
| XP_001389300 | Aspergillus niger |
| XP_457508 (DH61) | Debaryomyces hansenii CBS767 |
| XP_002551364 | Candida tropicalis MYA-3404 |
| XP_001523322 | Lodderomyces elongisporus NRRL |
| XP_720384 (29-4) | Candida albicans SC5314 |
| XP_456868 | Debaryomyces hansenii CBS767 |
| XP_001487429 (29-6) | Pichia guilliermondii ATCC 6260 |
| XP_961039 | Neurospora crassa |
| CAG88709 (DH48) | Debaryomyces hansenii CBS767 |
| XP_001727326 (29-9) | Aspergillus oryzae |
| XP_001816757 | Aspergillus oryzae |
| XP_457508 (DH61) | Debaryomyces hansenii CBS767 |
| XP_001727326 (29-9) | Aspergillus oryzae |
| XP_720384 (29-4) | Candida albicans SC5314 |
| XP_681669 (32-10) | Aspergillus nidulans FGSC A4 |
| XP_002488227 | Talaromyces stipitatus |
| AB070824.1 | Aspergillus oryzae |

In other embodiments, the pentose transporter is an arabinose transporter. Examples of suitable arabinose transporters include, without limitation, those listed in Table 8.

TABLE 8
Arabinose Transporters

| NCBI Reference | |
|---|---|
| Sequence | Organism |
| XP_002545773 | Candida tropicalis MYA-3404 |
| EEQ43601 | Candida albicans WO-1 |
| XP_001818631 | Aspergillus oryzae RIB40 |
| XP_002558275 | Penicillium chrysogenum Wisconsin 54-1255 |
| XP_001390883 | Aspergillus niger |
| XP_750103 | Aspergillus fumigatus Af293 |
| XP_960000 (NC52) | Neurospora crassa OR74A |
| XP_657854 (32-8) | Aspergillus nidulans FGSC A4 |
| XP_001825068 | Aspergillus oryzae RIB40 |
| XP_681669 (32-10) | Aspergillus nidulans FGSC |
| XP_002545773 | Candida tropicalis MYA-3404 |
| XP_657854 (32-8) | Aspergillus nidulans FGSC A4 |

In some embodiments, pentose transporters of the present disclosure include, without limitation, the xylose transporters NCU08221 and STL12/XUT6; the arabinose transporter XUT1; the arabinose/glucose transporter NCU06138; the xylose/glucose transporters SUT2, SUT3, and XUT3; the xylose/arabinose/glucose transporter NCU04963, homologs thereof; and orthologs thereof.

In other embodiments, host cells of the present disclosure further contain one or more recombinant enzymes involved in pentose utilization. The one or more enzymes may be endogenous or heterologous to the host cell. The one or more enzymes involved in pentose utilization may include, without limitation, L-arabinose isomerase, L-ribulokinase, L-ribulose-5-P 4 epimerase, xylose isomerase, xylulokinase, aldose reductase, L-arabitinol 4-dehydrogenase, L-xylulose reductase, and xylitol dehydrogenase in any combination. These enzymes may come from any organism that naturally metabolizes pentose sugars. Examples of such organisms include, without limitation, Kluyveromyces sp., Zymomonas sp., E. coli, Clostridium sp., and Pichia sp.

### Methods of Producing and Culturing Host Cells of the Present Disclosure

Other aspects of the present disclosure relate to the production of host cells containing one or more of a recombinant cellodextrin transporter, a recombinant cellodextrin phosphorylase, a recombinant β-glucosidase, a recombinant phosphoglucomutase, or a recombinant hexokinase. In certain embodiments, the host cell may further contain one or more glucose response genes of the present disclosure, one or more pentose transporters of the present disclosure, and/or one or more recombinant enzymes of the present disclosure involved in pentose utilization. Such host cells may be used to degrade cellodextrin and to produce hydrocarbons or hydrocarbon derivatives from cellodextrin.

Methods of producing and culturing host cells of the present disclosure may include the introduction or transfer of expression vectors containing recombinant polynucleotides into the host cell. Such methods for transferring expression vectors into host cells are well known to those of ordinary skill in the art. For example, one method for transforming E. coli with an expression vector involves a calcium chloride treatment where the expression vector is introduced via a calcium precipitate. Other salts, e.g., calcium phosphate, may also be used following a similar procedure. In addition, electroporation (i.e., the application of current to increase the permeability of cells to nucleic acid sequences) may be used to transfect the host cell. Also, microinjection of the nucleic acid sequences provides the ability to transfect host cells. Other means, such as lipid complexes, liposomes, and dendrimers, may also be employed. Those of ordinary skill in the art can transfect a host cell with a desired sequence using these or other methods.

The vector may be an autonomously replicating vector, i.e., a vector which exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one which, when introduced into the host, is integrated into the genome and replicated together with the chromosome(s) into which it has been integrated. Furthermore, a single vector or plasmid or two or more vectors or plasmids which together contain the total DNA to be introduced into the genome of the host, or a transposon may be used.

The vectors preferably contain one or more selectable markers which permit easy selection of transformed hosts. A selectable marker is a gene the product of which provides, for example, biocide or viral resistance, resistance to heavy metals, prototrophy to auxotrophs, and the like. Selection of bacterial cells may be based upon antimicrobial resistance that has been conferred by genes such as the amp, gpt, neo, and hyg genes.

Suitable markers for yeast hosts are, for example, ADE2, HIS3, LEU2, LYS2, MET3, TRP1, and URA3. Selectable markers for use in a filamentous fungal host include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in Aspergillus are the amdS and pyrG genes of Aspergillus nidulans or Aspergillus oryzae and the bar gene of Streptomyces hygroscopicus. Preferred for use in Trichoderma are bar and amdS.

The vectors preferably contain an element(s) that permits integration of the vector into the host's genome or autonomous replication of the vector in the cell independent of the genome.

For integration into the host genome, the vector may rely on the gene's sequence or any other element of the vector for integration of the vector into the genome by homologous or nonhomologous recombination. Alternatively, the vector may contain additional nucleotide sequences for directing integration by homologous recombination into the genome of the host. The additional nucleotide sequences enable the vector to be integrated into the host genome at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 10,000 base pairs, which are highly homologous with the corresponding target sequence to enhance the probability of homologous recombination. The integrational elements may be any sequence that is homologous with the target sequence in the genome of the host. Furthermore, the integrational elements may be non-encoding or encoding nucleotide sequences. On the other hand, the vector may be integrated into the genome of the host by non-homologous recombination.

For autonomous replication, the vector may further contain an origin of replication enabling the vector to replicate autonomously in the host in question. The origin of replication may be any plasmid replicator mediating autonomous replication which functions in a cell. The term “origin of replication” or “plasmid replicator” is defined herein as a sequence that enables a plasmid or vector to replicate in vivo. Examples of origins of replication for use in a yeast host are the 2 micron origin of replication, ARS1, ARS4, the combination of ARS1 and CEN3, and the combination of ARS4 and CEN6. Examples of origins of replication useful in a filamentous fungal cell are AMA1 and ANS1 (Gems et al., 1991; Cullen et al., 1987; WO 00/24883). Isolation of the AMA1 gene and construction of plasmids or vectors containing the gene can be accomplished according to the methods disclosed in WO 00/24883.

For other hosts, transformation procedures may be found, for example, in Jeremiah D. Read, et al., Applied and Environmental Microbiology, August 2007, p. 5088-5096, for Kluyveromyces; in Osvaldo Delgado, et al., FEMS Microbiology Letters 132, 1995, 23-26, for Zymomonas; in U.S. Pat. No. 7,501,275 for Pichia sipitis; and in WO 2008/040387 for Clostridium.

More than one copy of a gene may be inserted into the host to increase production of the gene product. An increase in the copy number of the gene can be obtained by integrating at least one additional copy of the gene into the host genome or by including an amplifiable selectable marker gene with the nucleotide sequence where cells containing amplified copies of the selectable marker gene, and thereby additional copies of the gene, can be selected for by cultivating the cells in the presence of the appropriate selectable agent.

The procedures used to ligate the elements described above to construct the recombinant expression vectors of the present disclosure are well known to one skilled in the art (see, e.g., Sambrook et al., 1989, supra).

The host cell is transformed with at least one expression vector. When only a single expression vector is used (without the addition of an intermediate), the vector will contain all of the nucleic acid sequences necessary.

Once the host cell has been transformed with the expression vector, the host cell is allowed to grow. Methods of the invention may include culturing the host cell such that recombinant nucleic acids in the cell are expressed. For microbial hosts, this process entails culturing the cells in a suitable medium. Typically cells are grown at 35° C. in appropriate media. Preferred growth media in the present invention include, for example, common commercially prepared media such as Luria Bertani (LB) broth, Sabouraud Dextrose (SD) broth or Yeast medium (YM) broth. Other defined or synthetic growth media may also be used and the appropriate medium for growth of the particular host cell will be known by someone skilled in the art of microbiology or fermentation science. Temperature ranges and other conditions suitable for growth are known in the art (see, e.g., Bailey and Ollis 1986).

According to some aspects of the present disclosure, the culture media contains a carbon source for the host cell. Such a “carbon source” generally refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, etc. These include, for example, various monosaccharides such as glucose, oligosaccharides such as cellodextrins, polysaccharides, a biomass polymer such as cellulose or hemicellulose, xylose, arabinose, disaccharides, such as sucrose, saturated or unsaturated fatty acids, succinate, lactate, acetate, ethanol, etc., or mixtures thereof. The carbon source can additionally be a product of photosynthesis, including, but not limited to glucose.

Lignocellulosic biomass is composed of cellulose, hemicellulose, and lignin. In some embodiments, the carbon source is a biomass polymer such as cellulose or hemicellulose. A “biomass polymer” as described herein is any polymer contained in biological material. The biological material may be living or dead. A biomass polymer includes, for example, cellulose, xylan, xylose, hemicellulose, lignin, mannan, and other materials commonly found in biomass. Non-limiting examples of sources of a biomass polymer include grasses (e.g., switchgrass, Miscanthus), rice hulls, bagasse, cotton, jute, hemp, flax, bamboo, sisal, abaca, straw, leaves, grass clippings, corn stover, corn cobs, distillers grains, legume plants, sorghum, sugar cane, sugar beet pulp, wood chips, sawdust, and biomass crops (e.g., Crambe).

In addition to an appropriate carbon source, media must contain suitable minerals, salts, cofactors, buffers and other components, known to those skilled in the art, suitable for the growth of the cultures and promotion of the enzymatic pathways necessary for the fermentation of various sugars and the production of hydrocarbons and hydrocarbon derivatives. Reactions may be performed under aerobic or anaerobic conditions where aerobic, anoxic, or anaerobic conditions are preferred based on the requirements of the microorganism. As the host cell grows and/or multiplies, expression of the enzymes, transporters, or other proteins necessary for growth on various sugars or biomass polymers, sugar fermentation, or synthesis of hydrocarbons or hydrocarbon derivatives is affected.

### Methods of Co-Fermentation

Other aspects of the present disclosure relate to methods of co-fermenting cellulose-derived and hemicellulose-derived sugars. As used herein, co-fermentation refers to simultaneous utilization by a host cell of more than one sugar in the same vessel. The method includes the steps of providing a host cell, where the host cell contains a recombinant cellodextrin transporter of the present disclosure, and one or more of a recombinant cellodextrin phosphorylase of the present disclosure, a recombinant β-glucosidase of the present disclosure, a recombinant phosphoglucomutase of the present disclosure, or a recombinant hexokinase of the present disclosure; and culturing the host cell in a medium containing a cellulose-derived sugar and a hemicellulose-derived sugar under conditions whereby the host cell co-ferments the cellulose-derived sugar and the hemicellulose-derived sugar. Any host cell described herein and containing a recombinant cellodextrin transporter of the present disclosure, and one or more of a recombinant cellodextrin phosphorylase of the present disclosure, a recombinant β-glucosidase of the present disclosure, a recombinant phosphoglucomutase of the present disclosure, or a recombinant hexokinase of the present disclosure may be used. In certain embodiments, the host cell may further contain one or more glucose response genes of the present disclosure, one or more pentose transporters of the present disclosure, and/or one or more recombinant enzymes of the present disclosure involved in pentose utilization.

In certain embodiments, the host cell may also contain at least one recombinant pentose transporter and one or more recombinant enzymes involved in pentose utilization. Alternatively, the at least one pentose transporter and one or more enzymes involved in pentose utilization may be endogenous to the host cell. The one or more enzymes may include, without limitation, L-arabinose isomerase, L-ribulokinase, L-ribulose-5-P 4 epimerase, xylose isomerase, xylulokinase, aldose reductase, L-arabitinol 4-dehydrogenase, L-xylulose reductase, xylitol dehydrogenase, or any other pentose-utilizing enzymes known to one of skill in the art.

In methods of co-fermentation as described herein, cellulose-derived sugars may include, without limitation, cellobiose, cellotriose, cellotetraose, etc.; and hemicellulose-derived sugars may include, without limitation, xylose and arabinose. Typically, in order to prepare the cellulose-derived sugars and hemicellulose-derived sugars for co-fermentation by a host cell, lignocellulosic biomass is first pretreated to alter its structure and allow for better enzymatic hydrolysis of cellulose. Pretreatment may include physical or chemical methods, including, for example, ammonia fiber/freeze explosion, the lime method based on calcium or sodium hydroxide, and steam explosion with or without an acid catalyst. Acid treatment will release xylose and arabinose from the hemicellulose component of the lignocellulosic biomass. Next, preferably, the cellulose component of the pretreated biomass is hydrolyzed by a mixture of cellulases. Examples of commercially available cellulase mixtures include Celluclast 1.5L® (Novozymes), Spezyme CP® (Genencor) (Scott W. Pryor, 2010, Appl Biochem Biotechnol), and Cellulyve 50L (Lyven).

### Methods of Degrading Cellodextrin

Other aspects of the present disclosure provide methods for degrading cellodextrin in a host cell. In one aspect, the present disclosure provides a method of degrading cellodextrin, by providing a host cell containing two or more of a recombinant cellodextrin transporter of the present disclosure, a recombinant cellodextrin phosphorylase of the present disclosure, a recombinant β-glucosidase of the present disclosure, a recombinant phosphoglucomutase of the present disclosure, or a recombinant hexokinase of the present disclosure; and culturing the host cell in a medium containing cellodextrin or a source of cellodextrin, whereby cellodextrin is degraded. Any host cell described herein and containing two or more of a recombinant cellodextrin transporter of the present disclosure, a recombinant cellodextrin phosphorylase of the present disclosure, a recombinant β-glucosidase of the present disclosure, a recombinant phosphoglucomutase of the present disclosure, or a recombinant hexokinase of the present disclosure may be used. In certain embodiments, the host cell may further contain one or more glucose response genes of the present disclosure, one or more pentose transporters of the present disclosure, and/or one or more recombinant enzymes of the present disclosure involved in pentose utilization.

In some embodiments, the source of cellodextrin is lignocellulosic biomass, which contains cellulose, hemicellulose, and lignin. In other embodiments, the source of cellodextrin is hemicellulose. In certain preferred embodiments, the source of cellodextrin is cellulose. Typically, the cellodextrin is cellobiose, cellotriose, cellotetraose, cellopentose, or cellohexose. Transport of cellodextrin into the cell may be measured by any methods known to one of skill in the art, including the methods described in US 2011/0020910.

Culturing conditions sufficient for the host cell to degrade cellodextrin are well known in the art and include any suitable culturing conditions disclosed herein. Typically, in order to prepare the cellodextrin or source of cellodextrin contained in the culture medium for utilization by the host cell, lignocellulosic biomass is first pretreated to alter its structure and allow for better enzymatic hydrolysis of cellulose. Pretreatment may include physical or chemical methods, including, for example, ammonia fiber/freeze explosion, the lime method based on calcium or sodium hydroxide, and steam explosion with or without an acid catalyst. Next, preferably, the cellulose component of the pretreated biomass is hydrolyzed by a mixture of cellulases. Examples of commercially available cellulase mixtures include Celluclast 1.5L® (Novozymes), Spezyme CP® (Genencor) (Scott W. Pryor, 2010, Appl Biochem Biotechnol), and Cellulyve 50L (Lyven).

### Methods of Synthesis of Hydrocarbons or Hydrocarbon Derivatives

Further aspects of the present disclosure provide methods for producing hydrocarbons or hydrocarbon derivatives from cellodextrin.

As used herein, “hydrocarbons” are organic compounds consisting entirely of hydrogen and carbon. Hydrocarbons include, without limitation, methane, ethane, ethene, ethyne, propane, propene, propyne, cyclopropane, allene, butane, isobutene, butene, butyne, cyclobutane, methylcyclopropane, butadiene, pentane, isopentane, neopentane, pentene, pentyne, cyclopentane, methylcyclobutane, ethylcyclopropane, pentadiene, isoprene, hexane, hexene, hexyne, cyclohexane, methylcyclopentane, ethylcyclobutane, propylcyclopropane, hexadiene, heptane, heptene, heptyne, cycloheptane, methylcyclohexane. heptadiene, octane, octene, octyne, cyclooctane, octadiene, nonane, nonene, nonyne, cyclononane, nonadiene, decane, decene, decyne, cyclodecane, and decadiene.

As used herein, “hydrocarbon derivatives” are organic compounds of carbon and at least one other element that is not hydrogen. Hydrocarbon derivatives include, without limitation, alcohols (e.g., arabinitol, butanol, ethanol, glycerol, methanol, 1,3-propanediol, sorbitol, and xylitol); organic acids (e.g., acetic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, and xylonic acid); esters; ketones (e.g., acetone); aldehydes (e.g., furfural); amino acids (e.g., aspartic acid, glutamic acid, glycine, lysine, serine, and threonine); and gases (e.g., carbon dioxide and carbon monoxide).

In preferred embodiments, the hydrocarbons or hydrocarbon derivatives can be used as fuel. In particularly preferred embodiments, the hydrocarbon or hydrocarbon derivative is ethanol or butanol.

In some embodiments, the hydrocarbon or hydrocarbon derivative is ethanol. In certain embodiments, the ethanol is produced at a rate that ranges from at least 0.10 to at least 50 g/L-h, from at least 0.1 to at least 40 g/L-h, from at least 0.1 to at least 30 g/L-h, from at least 0.1 to at least 20 g/L-h, from at least 0.1 to at least 10 g/L-h, from at least 0.1 to at least 5 g/L-h, from at least 0.1 to at least 1 g/L-h, from at least 0.5 to at least 40 g/L-h, from at least 0.5 to at least 20 g/L-h, from at least 0.5 to at least 10 g/L-h, from at least 0.5 to at least 5 g/L-h, from at least 0.5 to at least 1 g/L-h, from at least 1 to at least 40 g/L-h, from at least 1 to at least 20 g/L-h, from at least 1 to at least 10 g/L-h, from at least 1 to at least 5 g/L-h, from at least 5 to at least 40 g/L-h, from at least 5 to at least 20 g/L-h, from at least 5 to at least 10 g/L-h, from at least 10 to at least 50 g/L-h, from at least 10 to at least 40 g/L-h, or from at least 10 to at least 20 g/L-h.

In other embodiments, the ethanol is produced at a rate of about 0.10, about 0.15, about 0.20, about 0.25, about 0.30, about 0.35, about 0.40, about 0.45, about 0.50, about 0.55, about 0.60, about 0.65, about 0.70, about 0.75, about 0.80, about 0.85, about 0.90, about 0.95, about 1.00, about 1.25, about 1.50, about 1.75, about 2.00, about 2.25, about 2.50, about 2.75, about 3.00, about 3.25, about 3.50, about 3.75, about 4.00, about 4.25, about 4.50, about 4.75, about 5.00, about 5.25, about 5.50, about 5.75, about 6.00, about 6.25, about 6.50, about 6.75, about 7.00, about 7.25, about 7.50, about 7.75, or about 8.00, about 8.25, about 8.50, about 8.75, about 9.00, about 9.25, about 9.50, about 9.75, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, about 25, about 25.5, about 26, about 27, about 28, about 29, about 30, about 35, about 40, about 45, about 50, or more g/L-h. It should be noted that the rates of ethanol production described herein may vary by ±0.02 g/L-h. For example a rate of about 10 g/L-h could vary from 9.98 g/L-h to 10.02 g/L-h.

According to one aspect of the present disclosure, a method for producing hydrocarbons or hydrocarbon derivatives from cellodextrin, includes the steps of providing a host cell containing two or more of a recombinant cellodextrin transporter of the present disclosure, a recombinant cellodextrin phosphorylase of the present disclosure, a recombinant β-glucosidase of the present disclosure, a recombinant phosphoglucomutase of the present disclosure, or a recombinant hexokinase of the present disclosure; and culturing the host cell in a medium containing cellodextrin or a source of cellodextrin, whereby the host cell produces hydrocarbons or hydrocarbon derivatives from the cellodextrin. Any host cell described herein and containing two or more of a recombinant cellodextrin transporter of the present disclosure, a recombinant cellodextrin phosphorylase of the present disclosure, a recombinant β-glucosidase of the present disclosure, a recombinant phosphoglucomutase of the present disclosure, or a recombinant hexokinase of the present disclosure may be used. In certain embodiments, the host cell may further contain one or more glucose response genes of the present disclosure, one or more pentose transporters of the present disclosure, and/or one or more recombinant enzymes of the present disclosure involved in pentose utilization.

In some embodiments, the source of cellodextrin is lignocellulosic biomass, which contains cellulose, hemicellulose, and lignin. In other embodiments, the source of cellodextrin is hemicellulose. In certain preferred embodiments, the source of cellodextrin is cellulose. Typically, the cellodextrin is cellobiose, cellotriose, cellotetraose, cellopentose, or cellohexose.

### Methods of Reducing ATP Consumption During Glucose Utilization

Other aspects of the present disclosure provide methods for reducing ATP consumption during glucose utilization in a host cell. In one aspect, the present disclosure provides a method for reducing ATP consumption during glucose utilization, by providing a host cell containing one or more of a recombinant cellodextrin transporter of the present disclosure, a recombinant phosphoglucomutase of the present disclosure, or a recombinant hexokinase of the present disclosure, and containing a recombinant polypeptide containing Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), where the recombinant polypeptide has cellodextrin phosphorylase activity; and culturing the host cell in a medium containing cellodextrin or a source of cellodextrin, whereby cellodextrin is degraded by the recombinant polypeptide to glucose-1-phosphate, where the production of glucose-1-phosphate from cellodextrin reduces ATP consumption as compared to a corresponding cell lacking the recombinant polypeptide. Any host cell described herein and containing one or more of a recombinant cellodextrin transporter of the present disclosure, a recombinant phosphoglucomutase of the present disclosure, or a recombinant hexokinase of the present disclosure together with a recombinant cellodextrin transporter of the present disclosure may be used. In certain embodiments, the host cell may further contain one or more glucose response genes of the present disclosure, one or more pentose transporters of the present disclosure, and/or one or more recombinant enzymes of the present disclosure involved in pentose utilization.

Host cells containing a recombinant polypeptide having cellodextrin phosphorylase activity reduced ATP consumption; as the cellodextrin phosphorylase utilizes inorganic phosphate to phosphorolytic cleave cellodextrin to glucose-1-P, which can then be converted to glucose-6-phosphate by a phosphoglucomutase. In contrast, hydrolytic pathways for degrading cellodextrin, such as those that utilize a β-glucosidase, produce glucose, which then needs to be converted to glucose-6-phosphate by utilizing ATP as a phosphate donor. Thus, utilizing a cellodextrin phosphorylase saves 1 ATP equivalent per cleavage reaction, which reduces the amount of ATP that must be consumed to phosphorylate cellodextrin-derived glucose before it is utilized via glycolysis. ATP consumption may be measured by any methods known to one of skill in the art, and include any suitable methods disclosed herein.

In some embodiments, the source of cellodextrin is lignocellulosic biomass, which contains cellulose, hemicellulose, and lignin. In other embodiments, the source of cellodextrin is hemicellulose. In certain preferred embodiments, the source of cellodextrin is cellulose. Typically, the cellodextrin is cellobiose, cellotriose, cellotetraose, cellopentose, or cellohexose.

It is to be understood that, while the present disclosure has been described in conjunction with the preferred specific embodiments thereof, the foregoing description is intended to illustrate and not limit the scope of the present disclosure. Other aspects, advantages, and modifications within the scope of the present disclosure will be apparent to those skilled in the art to which the present disclosure pertains.

The following examples are offered to illustrate provided embodiments and are not intended to limit the scope of the present disclosure.

### EXAMPLES

### Example 1

### Introduction

There is considerable interest in engineering microbes to convert the sugars found in plant cell walls to fuels and other chemicals. Plant cell walls are composed of cellulose (a polymer of glucose), hemicellulose (a heterogeneous polymer of pentoses, hexoses and sugar acids), and lignin (a heterogeneous phenolic polymer). They are abundant in agricultural and municipal wastes, and in dedicated energy crops. The yeast, Saccharomyces cerevisiae, is a favored platform for these engineering efforts because it is robust, simple to manipulate genetically, and capable of high carbon fluxes. Despite this, S. cerevisiae has a number of deficiencies including an inability to naturally ferment pentose sugars, sensitivity to solvents, and sensitivity to inhibitory compounds found in deconstructed plant material.

Another deficiency is that S. cerevisiae does not naturally ferment cellodextrins such as cellobiose. Cellodextrins are short polymers of β (1→4) linked glucose, are the repeating unit of cellulose, and are produced by the enzymatic digestion of cellulose by cellulases. To allow cellodextrin consumption, S. cerevisiae has been modified to either secrete or surface-display a f-glucosidase to hydrolyze cellodextrins to glucose extracellularly; or to import cellodextrins with a cellodextrin transporter for intracellular hydrolysis by a β-glucosidase.

In the two hydrolytic pathways, the o-glycosidic linkages of cellodextrins are cleaved by hydrolases and H₂O to produce glucose; while in the phosphorolytic pathway they are cleaved by phosphorylases and inorganic phosphate (Pᵢ) to produce glucose and glucose-1-phosphate. This difference is significant, because the first step of the Embden-Meyerhof glycolytic pathway consumes ATP to phosphorylate glucose. Thus the phosphorolytic pathway may be preferable when ATP is in short supply because less ATP is consumed for glucose phosphorylation.

Pathways with low ATP demands may be preferable for fuel and chemical production from lignocellulosic substrates. Lignocellulose is commonly treated with dilute acid to hydrolyze hemicellulose and liberate cellulose from lignin. Enzymatic hydrolysis of cellulose results in a low pH hydrolyzate containing not only hexose and pentose sugars, but also high concentrations of hemicellulose-derived acetic acid. At low pH, acetic acid moves freely across the plasma membrane of S. cerevisiae and into the cytosol, where it deprotonates into acetate. To maintain homeostasis, the dissociated proton and acetate must be exported through the membrane-bound H⁺ pump, Pma1, and the weak acid efflux pump, Pdr12, both of which consume ATP.

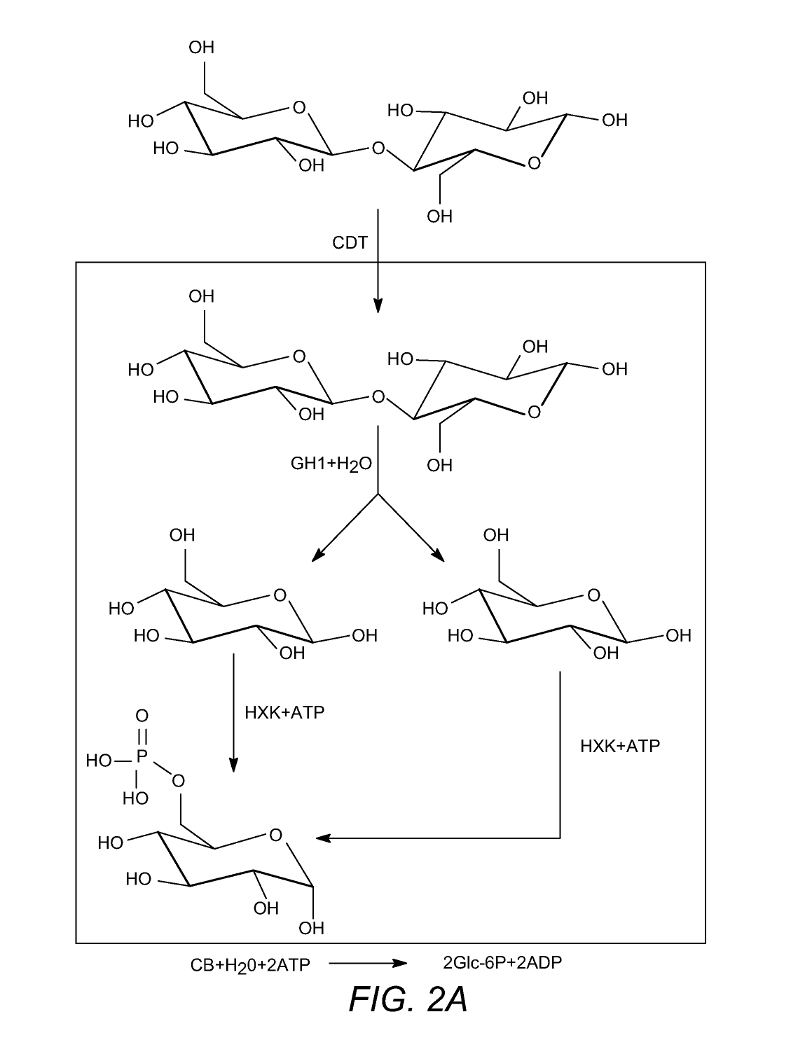

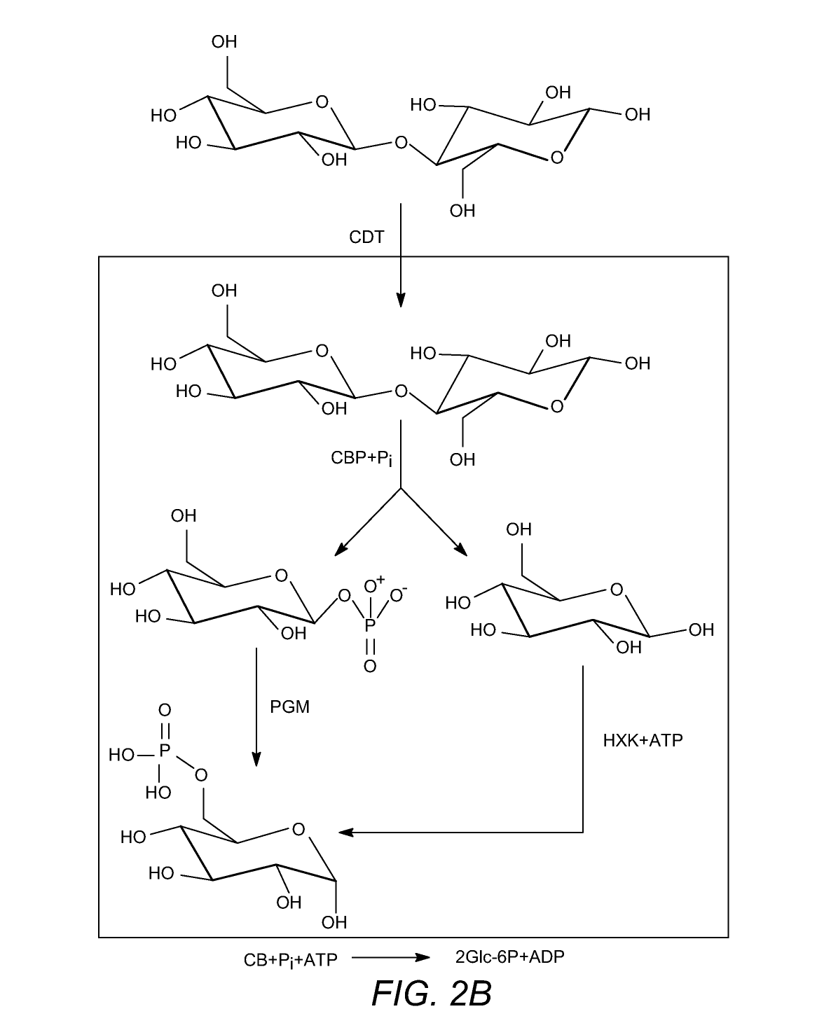

Accordingly, the following Example compares the performance of two cellobiose fermentation pathways that differ only in the mechanism by which imported cellobiose is cleaved (FIG. 2). The first pathway employs an intracellular hydrolytic enzyme, while the second pathway employs an intracellular phosphorolytic enzyme (FIG. 2).

Materials and Methods

Cellobiose Phosphorylase and Phosphoglucomutase Cloning

Cellobiose phosphorylase (CBP) genes from Celvibrio gilvus (CgCBP, Accession: AB010707), Sacharophagus degradans (SdCBP, Accession: YP_{—}526792), and Clostridium thermocellum (CtCBP, Accession: YP_{—}001036707) were codon-optimized, and synthesized by DNA2.0. The genes were inserted between the restriction sites SpeI and PstI in the 2μ plasmid pRS425, which had been previously modified to include the S. cerevisiae PGK1 promoter and the Cyc transcriptional terminator (PGK1_pRS426).

The C. gilvus CBP gene was inserted into the PGK1_pRS426 plasmid to create the plasmid PGK1_CgCBP_{—}425. The C. gilvus CBP gene was inserted using the following primers:

(SEQ ID NO: 201)
5′-TTA CTA GTA TGG GAT CAT CTC ACC ACC-3′;
and
(SEQ ID NO: 202)
5′-ATT CTG CAG TTA ATG ATG ATG ATG ATG ATG TAC TGT
CAC TTC GAC TCT CAC AGT AG-3′

The S. degradans CBP gene was inserted into the PGK1_pRS426 plasmid to create the plasmid PGK1_SdCBP_{—}425. The S. degradans CBP gene was inserted using the following primers:

(SEQ ID NO: 203)
5′-TTA CTA GTA TGA AAT TCG GGC ACT TTG-3′;
and
(SEQ ID NO: 204)
5′-ATT CTG CAG TTA ATG ATG ATG ATG ATG ATG TCC AAG
TGT TAC CTC GAC ATT G-3′

The C. thermocellum CBP gene was inserted into the PGK1_pRS426 plasmid to create the plasmid PGK1_CtCBP_{—}425. The C. thermocellum CBP gene was inserted using the following primers:

(SEQ ID NO: 205)
5′-TTA CTA GTA TGA AGT TTG GCT TTT TCG ATG-3′;
and
(SEQ ID NO: 206)
5′-ATT CTG CAG TTA ATG ATG ATG ATG ATG ATG TCC AAG
TGT TAC CTC GAC ATT G-3′

For all primers disclosed herein, restriction sites are underlined, and 6×-hisitidine tags are italicized.

To construct plasmids containing both a CBP gene and the S. cerevisiae phosphoglucomutase gene, Pgm2 (Accession: CAA89741) was first cloned between the SpeI and PstI restriction sited in the plasmid PGK1_pRS426 to create the plasmid PGK1_PGM_{—}425. The Pgm2 gene was inserted using the following primers:

(SEQ ID NO: 207)
5′-TTACTAGTATGTCATTTCAAATTGAAACGGTTC-3′;
and
(SEQ ID NO: 208)
5′-ATTCTGCAGTTAAGTACGAACCGTTGGTTCTTC-3′

The Pgm2 gene bracketed by the PGK1 promoter and the Cyc transcriptional terminator was then amplified from PGK1_PGM_{—}425 plasmid using the following primers:

(SEQ ID NO: 209)
5′-ATGAGCTCTGAATAATACGACTCACTATAGGGCGAATTG-3′;
and
(SEQ ID NO: 210)
5′-ATGAGCTCTGAATGGAAACAGCTATGACCATGATTACG-3′

This fragment was then inserted into the SacI restriction site of the PGK1_SdCBP_pRS425 plasmid, the PGK1_CgCBP_{—}425 plasmid, and the PGK1_CtCBP_{—}425 plasmid; creating the plasmids PGK1_SdCBP_PGM_{—}425, PGK1_CgCBP_PGM_{—}425, and PGK1_CtCBP_PGM_{—}425.

S. cerevisiae Strain Construction and Growth

To create the yeast strains used in this study, plasmids were transformed into the S. cerevisiae strain D452-2 (MATα leu2 his3 ura3 can1) (Hosaka, 1992) using the yeast EZ-Transformation kit (BIO 101, Vista, Calif.). To select transformants using an amino acid auxotrophic marker, yeast synthetic complete (YSC) medium was used, which contained 6.7 g/L yeast nitrogen base plus 20 g/L glucose, 20 g/L agar, and CSM-Leu-Trp-Ura-His (Bio 101, Vista, Calif.). This medium supplied the appropriate nucleotides and amino acids.

Fermentation

A single colony from YSC plates was grown overnight in 5 mL of YP medium (10 g/L yeast extract and 20 g/L peptone) containing 20 g/L of cellobiose. Cells at mid-exponential phase were harvested and inoculated after washing twice by sterilized water. All of the flask fermentation experiments were performed using 50 mL of YP medium containing 80 g/L of cellobiose in 250 mL flask at 30° C. with initial OD₆₀₀ of ˜1.0 and under oxygen limited conditions. Cell growth was monitored by optical density (OD) at 600 nm using UV-visible Spectrophotometer (Biomate 5, Thermo, N.Y.). Ethanol, acetate, glucose, and glycerol concentrations were determined by high performance liquid chromatography (HPLC, Agilent Technologies 1200 Series) equipped with a refractive index detector using a Rezex ROA-Organic Acid H+ (8%) column (Phenomenex Inc., Torrance, Calif.). The column was eluted with 0.005 N of H₂SO₄ at a flow rate of 0.6 ml/min at 50° C.

Directed Evolution of Phosphorolytic Strains

A fermentation reaction was started as described above using the D452-2 strain of S. cerevisiae transformed with cdt-1 and the S. degradans CBP. When cellobiose concentrations reached almost zero, cells were collected and used to establish new reactions at an OD (600 nm) of ˜0.01. This process was repeated 7 times over the course of 30 days. At this point, cells were plated onto YSC plates containing 20 g/L of cellobiose to isolate clones. After the confirmation of improved phenotypes from isolated clones, plasmids were isolated from one of representative clone and sequenced.

Site Directed Mutagenesis and Transporter Kinetics

Site directed mutagenesis was performed using the Quickchange® protocol (Zheng et al., Nucleic Acids Res. 2004 Aug. 10; 32(14):e115). The primers used to introduce each mutation are listed in Table 9. Transporter kinetics were measured as described previously with slight modifications (Galazka et al., Science. 2010 Oct. 1; 330(6000):84-6. Epub 2010 Sep. 9). Briefly, the yeast strain D452-2 transformed with mutant transporters was set-up at an OD (600 nm) of −0.2 in 50 mL of DOB-uracil and grown to an OD (600 nm) of ˜1. Cells were then harvested by centrifugation, washed 3× with 10 mLs of transport buffer (30 mM MES-NaOH [pH 5.6], 50 mM EtOH), and resuspended to a final OD (600 nm) of ˜40 in transport buffer. The GFP fluorescence of 100 μL of these cells was determined with excitation/emission wavelengths of 485/535 nm Beckman Coulter Paradigm™ plate reader. To record linear rates of uptake over the course of 95 seconds, 50 μL of cells were added to 50 μL of [³H]-cellobiose at the appropriate concentration with a final S.A. of 40 μCi/μmol in transport buffer layered over 100 μL of silicone oil (Sigma 85419). Reactions were stopped by spinning cells through the oil for 1 minute at 17,000 g, tubes were frozen in ethanol/dry ice, and tube-bottoms containing the cell-pellets were clipped off into 1 mL of 0.5 M NaOH. The pellets were solubilized overnight, 5 mL of Ultima Gold scintillation fluid added, and CPM determined in a Tri-Carb 2900TR scintillation counter. [³H]-cellobiose was purchased from Moravek Biochemicals, Inc., and had a specific activity of 4 Ci/mmol and a purity of >99%. Vₘₐₓ and K_{M} values were determined by fitting a single rectangular, 2-parameter hyperbolic function to a plot of rates vs. cellobiose concentration by non-linear regression in SigmaPlot®.

TABLE 9

| | Sense primer | Antisense primer |
|---|---|---|
| Mutation | (Mutated codon in lowercase) | (Mutated codon in lowercase) |
| F213L | TCTACAACTGCGGTTGGttaGGAGGTTCGA | GGAATCGAACCTCCtaaCCAACCGCAGTTGT |
| | TTCC (SEQ ID NO: 211) | AGA (SEQ ID NO: 212) |
| G91A | TGCGCCAACGGTTACGATgcaTCACTCATG | GATGATTCCGGTCATGAGTGAtgcATCGTAA |
| | ACCGGAATCATC (SEQ ID NO: 213) | CCGTTGGCGCA (SEQ ID NO: 214) |
| F335A | GGTGCTCATGATCTCCATCgcaGGCCAGTT | GTTGCCGGAGAACTGGCCtgcGATGGAGATC |
| | CTCCGGCAAC (SEQ ID NO: 215) | ATGAGCACC (SEQ ID NO: 216) |
| Q104A | ATCATCGCTATGGACAAGTTCgcaAACCAA | GTCACCAGTGTGGAATTGGTTtgcGAACTTG |
| | TTCCACACTGGTGAC (SEQ ID NO: 217) | TCCATAGCGATGAT (SEQ ID NO: 218) |
| F170A | TCCTCCAAGCTCGCTCAGgcaGTCGTTGGC | AACGAAGCGGCCAACGACtgcCTGAGCGAG |
| | CGCTTCGTT (SEQ ID NO: 219) | CTTGGAGGA (SEQ ID NO: 220) |
| R174A | GCTCAGTTTGTCGTTGGCgcaTTCGTTCTTG | ACCGAGGCCAAGAACGAAtgcGCCAACGAC |
| | GCCTCGGT (SEQ ID NO: 221) | AAACTGAGC (SEQ ID NO: 222) |
| E194A | GCCCCGGCCTACTCCATCgcaATCGCCCCT | CCAGTGAGGAGGGGCGATtgcGATGGAGTA |
| | CCTCACTGG (SEQ ID NO: 223) | GGCCGGGGC (SEQ ID NO: 224) |

Measurement of GFP Fluorescence During Fermentations

During fermentation, when OD (600 nm) reached 10.0, cells were harvested and washed twice by sterilized water. 200 μL of the cell suspension was transferred to a Corning black 96-well optical bottom plate (Corning, N.Y.). Fluorescence intensities were measured with a Biotek Synergy HT spectrophotometer (Biotek, Winooski, Vt.) at an excitation wavelength of 485 nm, emission of 528 nm.

Measurement of Enzymatic Activity in Cell Extracts

Fermentation reactions were set-up as described above in either YPC80 or YPD80 medium (10 g/L yeast extract, 20 g/L peptone, 80 g/L glucose). At an OD (600 nm) of ˜10, which corresponds to the exponential phase of the reaction, 2 mL of the culture was removed and cells pelleted. The cell pellet was washed 2× in 500 μL of ice-cold extraction buffer (50 mM HEPES-NaOH [pH 6.0], 2 mM DTT, and Roche Complete EDTA-free Protease Inhibitor Cocktail), and resuspended in 200 μL of this buffer, this was moved to a screw-cap tube containing ˜100 μL of 0.4 mm Zirconia/Silica beads. Cells were then lysed by bead-beating 3× for 30 s at 4° C. using a Biospec Products Mini-BEADBEATER WITH 30 s pause between runs. Debris was then pelleted, and the concentration of protein in the supernatant determined by the Bradford assay using reagents and the microtiter plate protocol from Bio-Rad.

The amount of cellobiase activity in cell extracts was determined by the Glucose oxidase/Peroxidase assay. 10 μg of cell extract was added to 1 mL of an assay mixture consisting of 50 mM phosphate buffer [pH 6.0], 10 U glucose oxidase, 10 U peroxidase, 1 mM o-dianisidine, and 10 mM cellobiose. The number of pmol of glucose produced per second was calculated by multiplying the rate of increase at 436 nm by 1.17×10³, a conversion that was established from a glucose standard curve.

The amount of hexokinase activity in cell extracts was determined by coupling the production of glucose-6-phosphate to the reduction of NADP⁺ by glucose-6-phosphate dehydrogenase. 10 μg of cell extract was added to 1 mL of an assay mixture consisting of 50 mM Tris-HCl [pH 8.0 at 30° C.], 13.3 mM MgCl₂, 540 μM ATP, 20 μM NADP⁺, 1 U S. cerevisiae glucose-6-phosphate dehydrogenase, and 112 mM glucose. The number of pmol of glucose-6-phosphate produced per second was calculated by multiplying the rate of increase at 340 nm by 1.85×10³, a conversion that was established from a glucose-6-phosphate standard curve.

Purification of GH1-1, SdCBP, Hxk1, Hxk2, and Glk1

GH1-1 and SdCBP were purified directly from the D452-2 yeast strains described above. 200 mL cultures in DOB-uracil-leucine were grown to an OD (600 nm) of ˜7. Cells were pelleted and washed 1× with 40 mL ddH₂O. Cell pellets were resuspended in 25 mL of 50 mM NaH₂PO₄ [pH 8.0], 300 mM NaCl, 10 mM imidazole, 2 mM DTT, and Roche Complete EDTA-free Protease Inhibitor Cocktail, and lysed by passage through an Avestin EmulsiFlex C-3 homogenizer at 20,000 P.S.I. Cell debris was then pelleted, and proteins purified using Nickel-NTA agarose beads from Qiagen following the protocol for native batch purification provided by Qiagen. Protein eluted from the beads was buffer exchanged into a buffer consisting of phosphate buffered saline (PBS), 10% glycerol, and 2 mM DTT, snap-frozen in (1)N₂ and stored at −80° C. Protein concentrations were determined by the absorbance at 280 nm using extinction coefficients of 108750 M⁻¹ cm⁻¹ and 178540 M⁻¹ cm⁻¹, for GH1-1 and SdCBP, respectively.

The Hxk1 (Accession: NP_{—}116711), Hxk2 (Accession: NP_{—}011261), and Glk1 (Accession: NP_{—}009890) genes were expressed and purified in E. coli. First, the genes were cloned into the PmlI and XhoI restriction sites of the expression plasmid pET302. Hxk1 gene was amplified using the following primers:

(SEQ ID NO: 225)
5′-CAT TAA CAC GTG GTT CAT TTA GGT CCA AAG AAA CCA
C-3′; and
(SEQ ID NO: 226)
5′-CAT TAA CTC GAG CAA TGA TAC CAA GAG ACT TAC CTT
CG-3′

The Hxk2 gene was amplified using the following primers:

(SEQ ID NO: 227)
5′-CAT TAA CAC GTG GTT CAT TTA GGT CCA AAA AAA CCA
C-3′; and
(SEQ ID NO: 228)
5′-CAT TAA CTC GAG TTA AGC ACC GAT GAT ACC AAC
G-3′

The Glk1 gene was amplified using the following primers:

(SEQ ID NO: 229)
5′-CAT TAA CAC GTG TCA TTC GAC GAC TTA CAC AAA
GC-3′; and
(SEQ ID NO: 230)
5′-CAT TAA CTC GAG TCA TGC TAC AAG CGC ACA
C-3′

These constructs were transformed into the BL21(DE3) strain of E. coli, and the proteins were expressed and purified using Nickel-NTA agarose beads from Qiagen following the protocol for native batch purification provided by Qiagen. Protein eluted from the beads was buffer exchanged into a buffer consisting of 50 mM Tris-HCl [pH 8.0 at 30° C.], 13.3 mM MgCl₂, 2 mM DTT, and 10% glycerol, snap-frozen in (1)N₂ and stored at −80° C. Protein concentrations were determined by the absorbance at 280 nm using extinction coefficients of 45840 M⁻¹ cm⁻¹, 45840 M⁻¹ cm⁻¹ and 30370 M⁻¹ cm⁻¹, for Hxk1, Hxk2 and Glk1, respectively.

Transglycosylation Activity of GH1-1 and SdCBP

To measure the transglycosylation activity of GH1-1 and SdCBP, 100 nkat of each enzyme was incubated at 37° C. with 100 μL 20% (w/v) cellobiose in 50 mM phosphate buffer [pH 6.0] and 2 mM DTT. After 12 hours, reactions were quenched in 400 μL 0.1 M NaOH and analyzed by ion chromatography with a Dionex ICS-3000, using a CarboPac PA200 column. Peaks were detected with an electrochemical detector.

Kinetic Parameters of GH1-1 and SdCBP

The kinetic parameters of purified GH1-1 and SdCBP were determined by measuring the rate of glucose production at a variety of cellobiose concentrations using the glucose oxidase/peroxidase assay in a manner identical to that used on cell extracts detailed above. A 1 mL assay included either 8.75 μmol of GH1-1 or 20 μmol SdCBP. Vₘₐₓ and K_{M} values were determined by fitting a single rectangular, 2-parameter hyperbolic function to a plot of glucose production rates vs. cellobiose concentration by non-linear regression in SigmaPlot®.

Effect of Cellobiose on S. cerevisiae Hexokinases

To measure the effect of high concentrations of cellobiose on the activity of purified Hxk1p, Hxk2p, and Glk1p, the activity of the purified proteins was measured in the presence or absence of 184 mM cellobiose, by coupling the production of glucose-6-phosphate to NADP⁺ reduction through glucose-6-phosphate dehydrogenase in a manner identical to that used on cell extracts detailed above. Between 1 and 10 μmol of Hxk1, Hxk2, or Glk1 was used.

Results

Yeast Strains Expressing a Cellobiose Phosphorylase and a Cellodextrin Transporter

Cellobiose phosphorylase (CBP) genes from Saccharophagus degradans (SdCBP), Celvibrio gilvus (CgCBP), and Clostridium thermocellum (CtCBP) were codon optimized, synthesized and cloned into 2μ plasmids. These plasmids were transformed, along with a plasmid carrying the GFP-tagged cellodextrin transporter gene cdt-1, into S. cerevisiae strain D452-2.

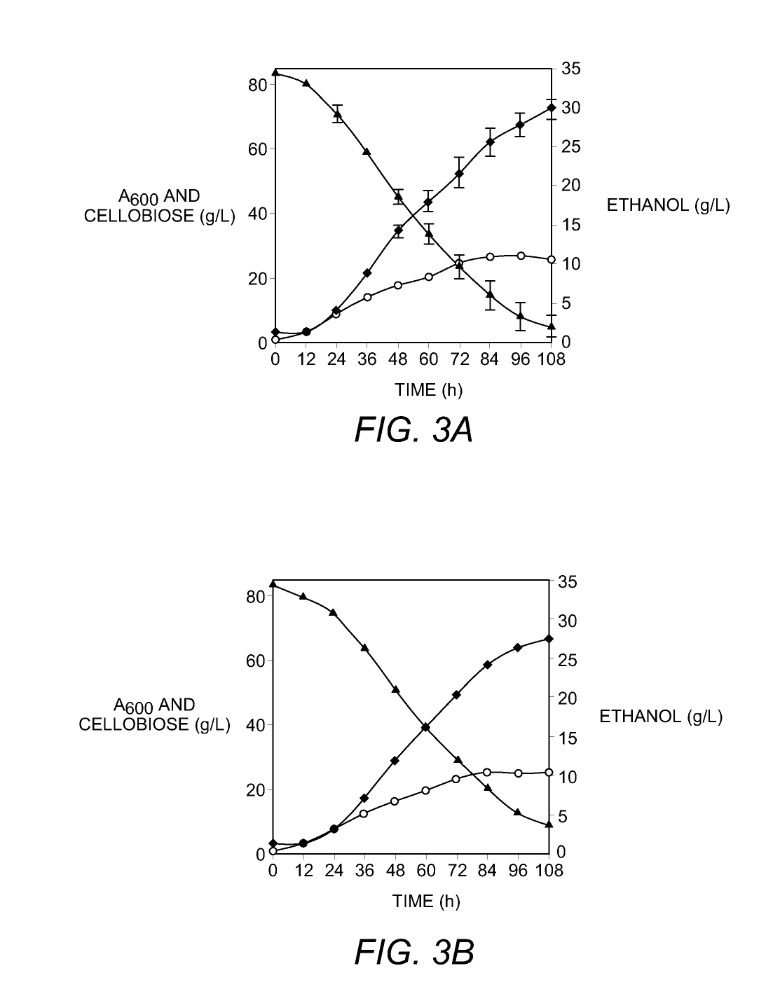

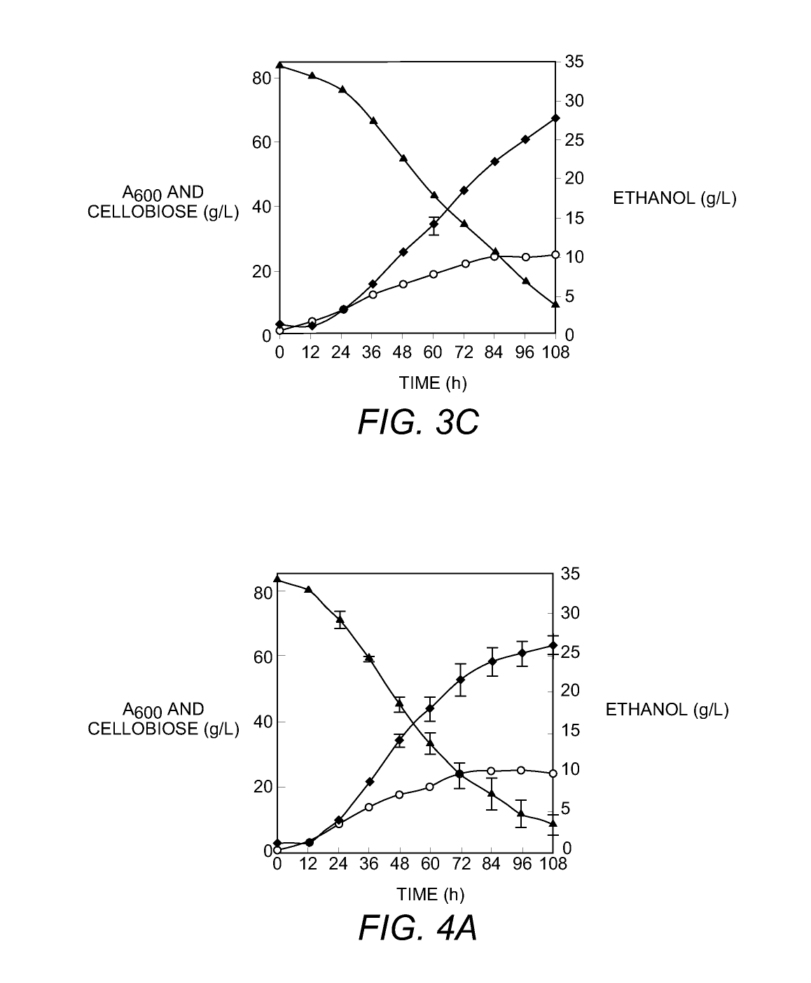

All three resulting strains consumed cellobiose and produced ethanol (FIG. 3). The cellobiose consumption rate of each of the three engineered strains was similar, ranging from about 0.95 to about 1.02 g/L-h. The ethanol production rates of the three strains were also similar, ranging from about 0.42 to about 0.44 g/L-h. After 77 hours, almost all of the cellobiose was fermented to ethanol by each of the three strains with a yield ranging from about 0.43 to about 0.45 g/g. Moreover, there was no appreciable accumulation of acetate, glucose, or glycerol.

Previously developed cellobiose-transporting yeast strains that rely on intracellular hydrolysis by β-glucosidase, ferment cellobiose with a concomitant accumulation of glucose and cellodextrins (e.g., cellotriose and cellotetraose) in the extracellular media. However, this phenomenon was not observed with the three yeast strains expressing a cellobiose phosphorylase rather than the β-glucosidase.

Ectopic Expression of Phosphoglucomutase

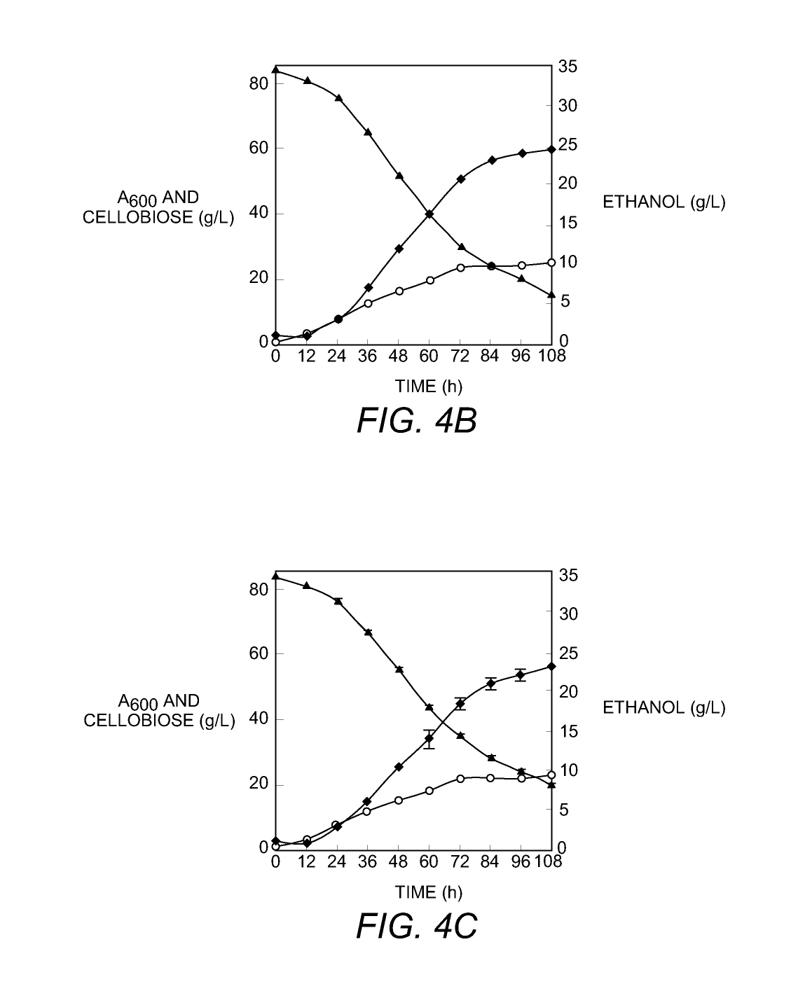

Cellobiose phosphorylase phosphorolytically cleaves cellobiose to glucose and glucose-1-phosphate, and glucose-1-phosphate needs to be converted to glucose-6-phosphate by a phosphoglucomutase (PGM) to enter the glycolytic pathway. As PGM is transcriptionally downregulated during glycolytic growth, a set of yeast strains in which the PGM of S. cerevisiae was expressed ectopically along with CDT-1 and CBP was tested to determine whether ectopic expression of PGM increases ethanol production. However, these strains showed slightly reduced performance compared to corresponding yeast strains that did not ectopically express PGM (FIG. 4).

Improved Phosphorolytic Yeast Strains

To improve upon the fermentation results of the engineered yeast strains expressing a cellodextrin transporter and a cellobiose phosphorylase, the strain expressing CDT-1 and SdCBP was enriched by serial transfer to YP medium containing 80 g/L of cellobiose for 30 days. An improved strain emerged that consumed cellobiose and produced ethanol 2-times faster than the parental strain. The ethanol productivity by evolved strain was 1.00 g/L-h while the parental strain exhibited 0.40 g/L-h of ethanol productivity.

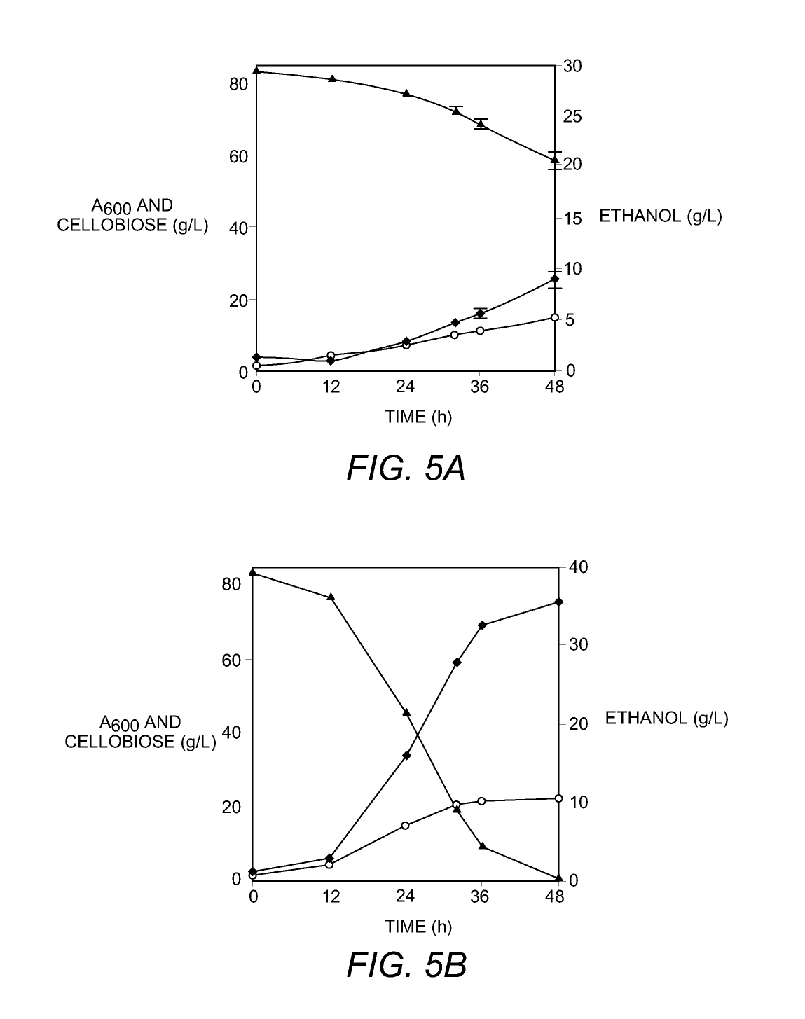

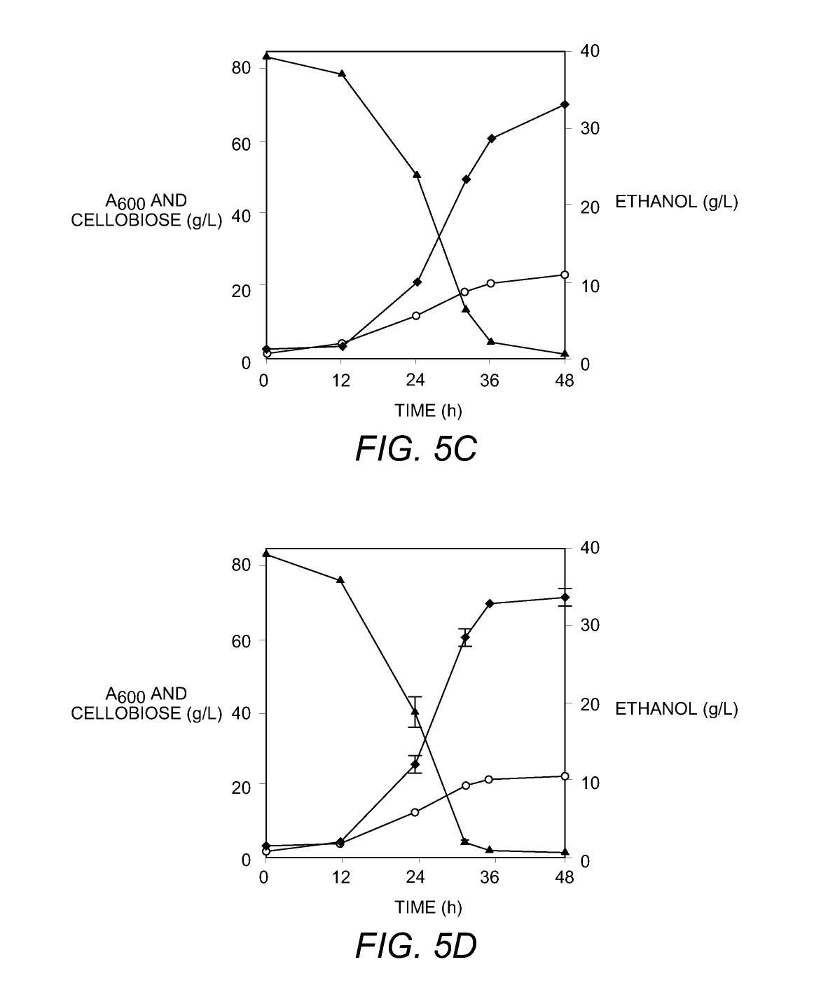

In order to identify the mutations responsible for the improved yeast strain, both 2μ plasmids (pRS425-SdCBP and pRS426-CDT-1) were isolated from the evolved strain and sequenced. The sequencing identified a single nucleotide mutation (C 639A) in the cdt-1 open reading frame, corresponding to a change of phenylalanine to leucine at position 213 (F213L) in the translated polypeptide. This single mutation is responsible for the improved performance, as retransforming the isolated plasmid into the native D452-2 yeast strain recapitulated the result (FIG. 5). The strains expressing the mutant CDT-1(F213L) and SdCBP consumed cellobiose at a rate of 2.06±0.04 g/L-h, and produced ethanol at a rate 0.90±0.01 g/L-h (FIG. 5B). This is an improvement of 102% in the cellobiose production rate and an improvement of 105% in the ethanol production rate. Ethanol yields were unaffected, and there was no appreciable accumulation of, acetate, glucose, glycerol, or cellodextrins.

Mutant CDT-1 (F213L) Expression in Conjunction with β-Glucosidase Expression

Strains expressing CDT-1 with an intracellular β-glucosidase were previously shown to ferment cellobiose to ethanol. In order to assess whether the mutant CDT-1 (F213L) would also improve the performance of such a strain, the mutant CDT-1 (F213L) was expressed in D452-2 along with the intracellular β-glucosidase GH1-1. Only a slight improvement in cellobiose fermentation was seen compared to a strain expressing WT CDT-1 and GH1-1 (FIGS. 5C and 5D).

As previously reported (Ha et al., Proc Natl Acad Sci USA. 2011 Feb. 15; 108(7):2735-40. Epub 2011 Jan. 31), cellobiose fermentation by strains expressing CDT-1 and GH1-1 occurs with an accumulation of glucose and cellodextrins, and the same pattern was seen in the strain expressing GH1-1 and CDT-1 F213L.

Cellodextrin Transporter Mutants

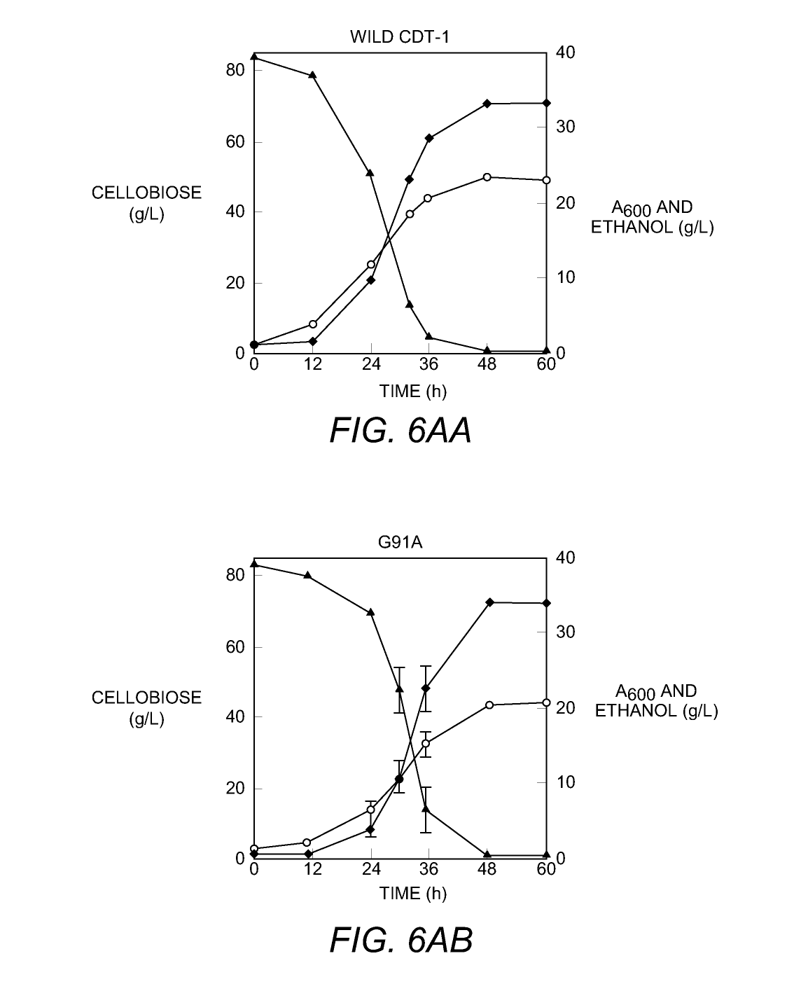

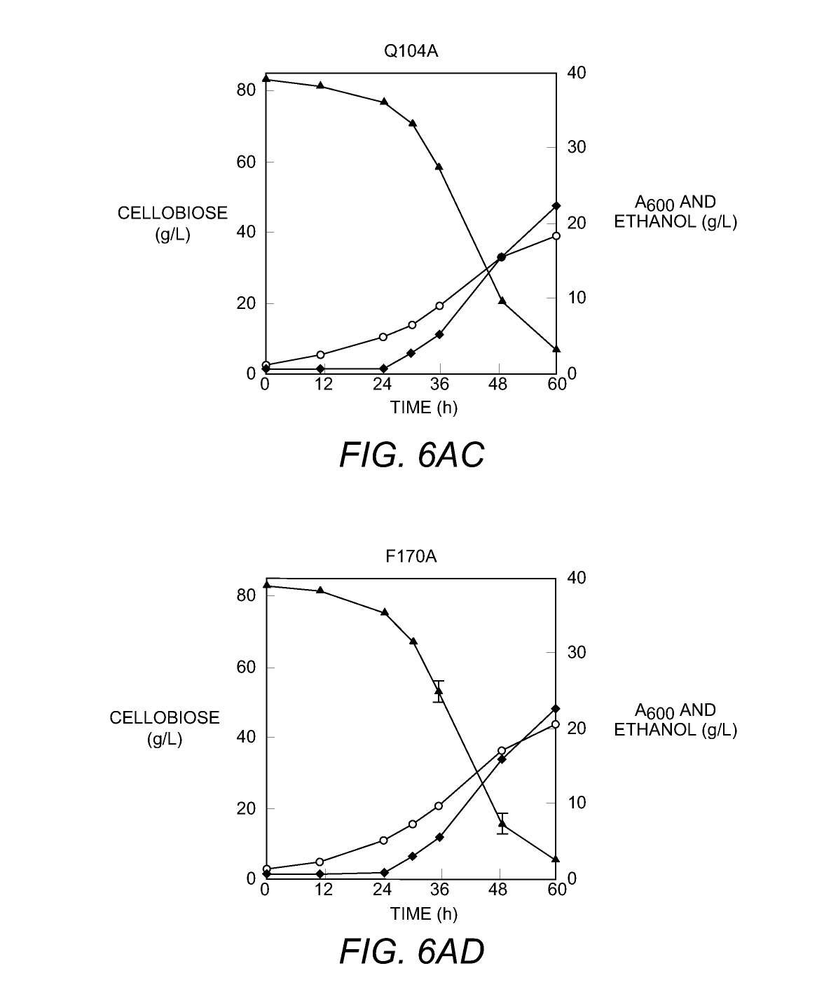

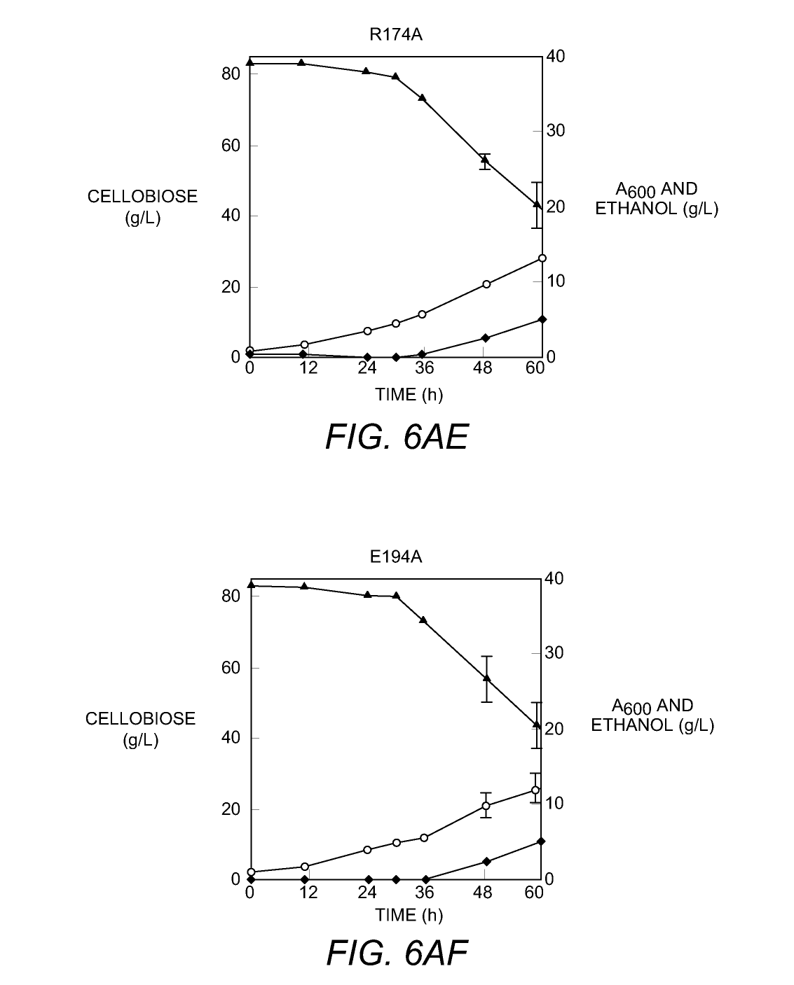

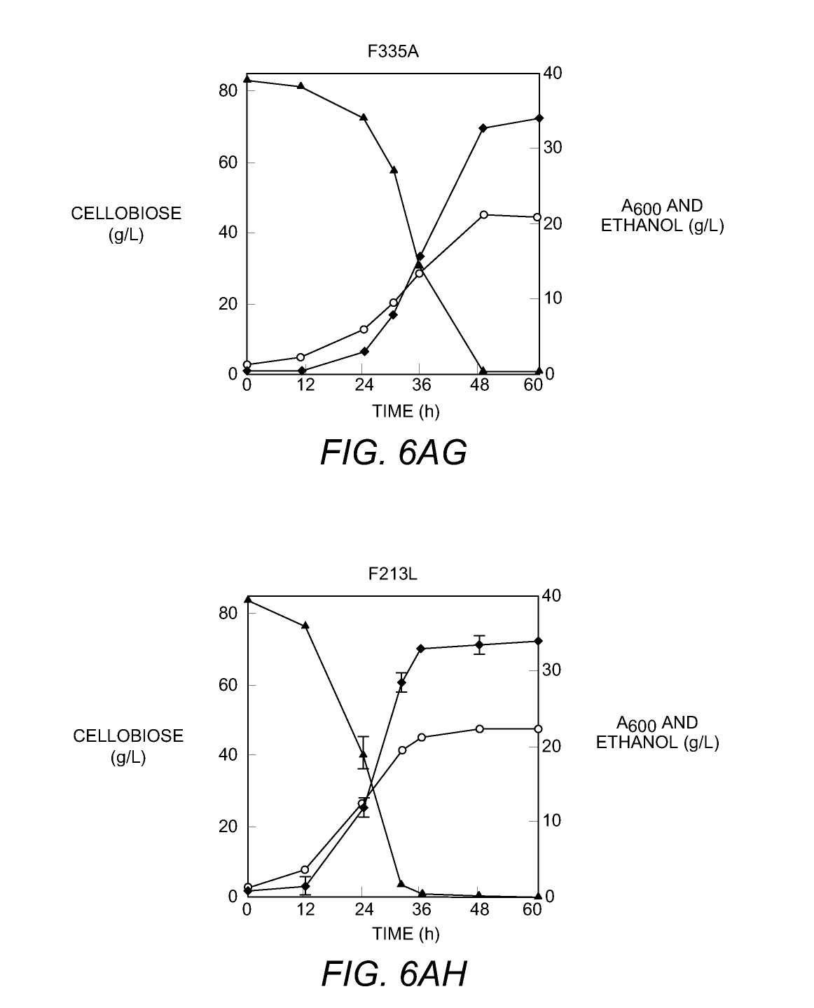

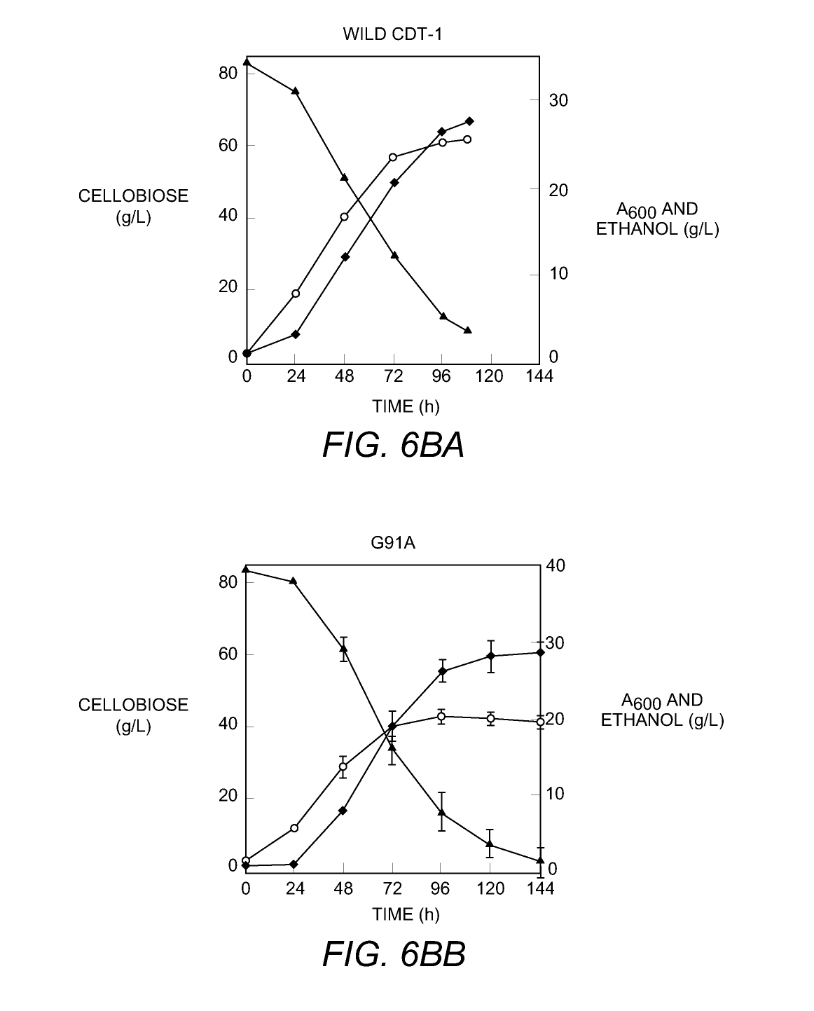

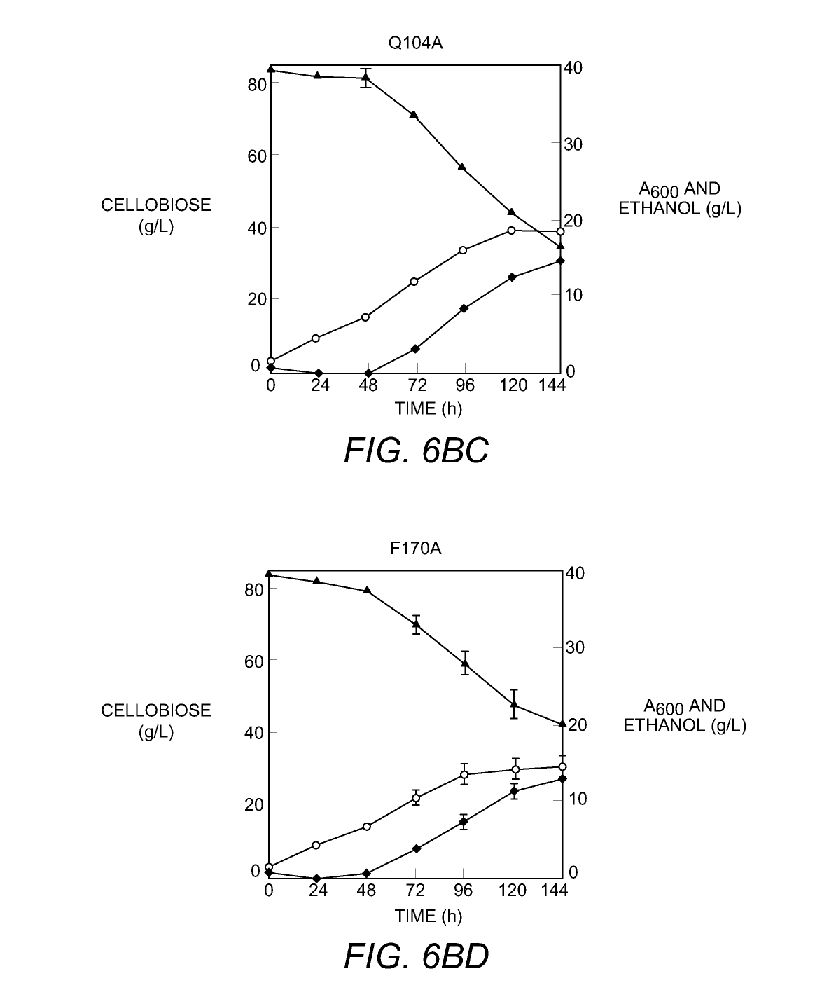

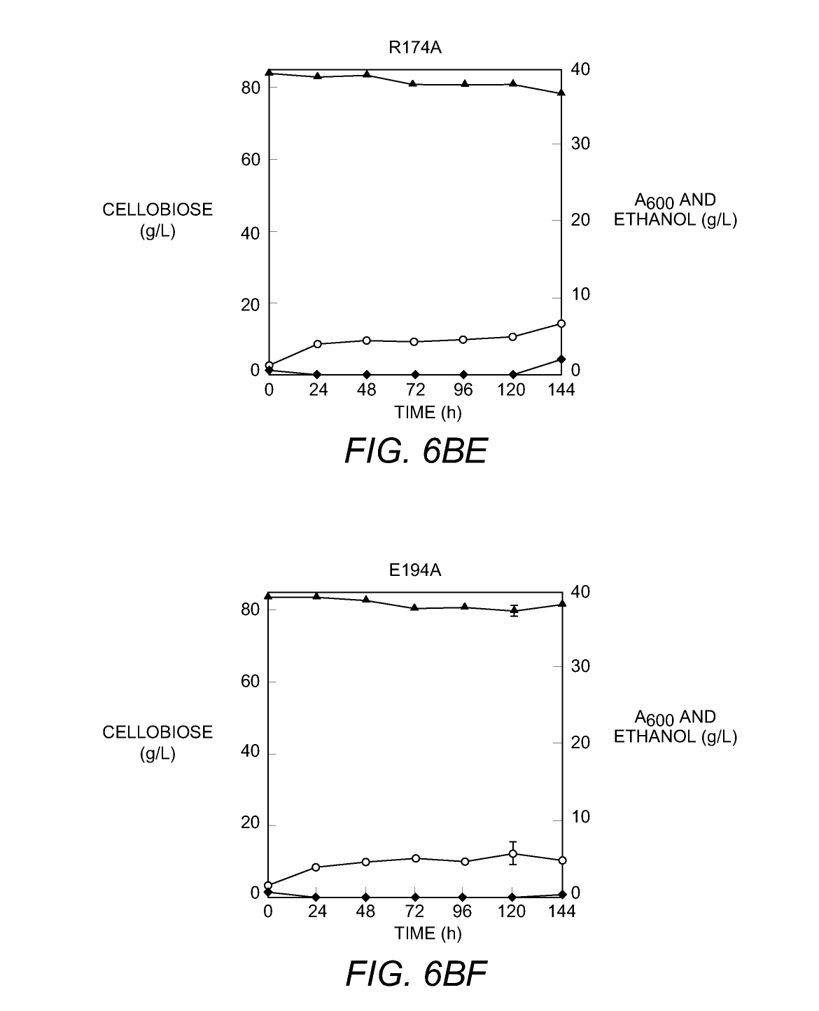

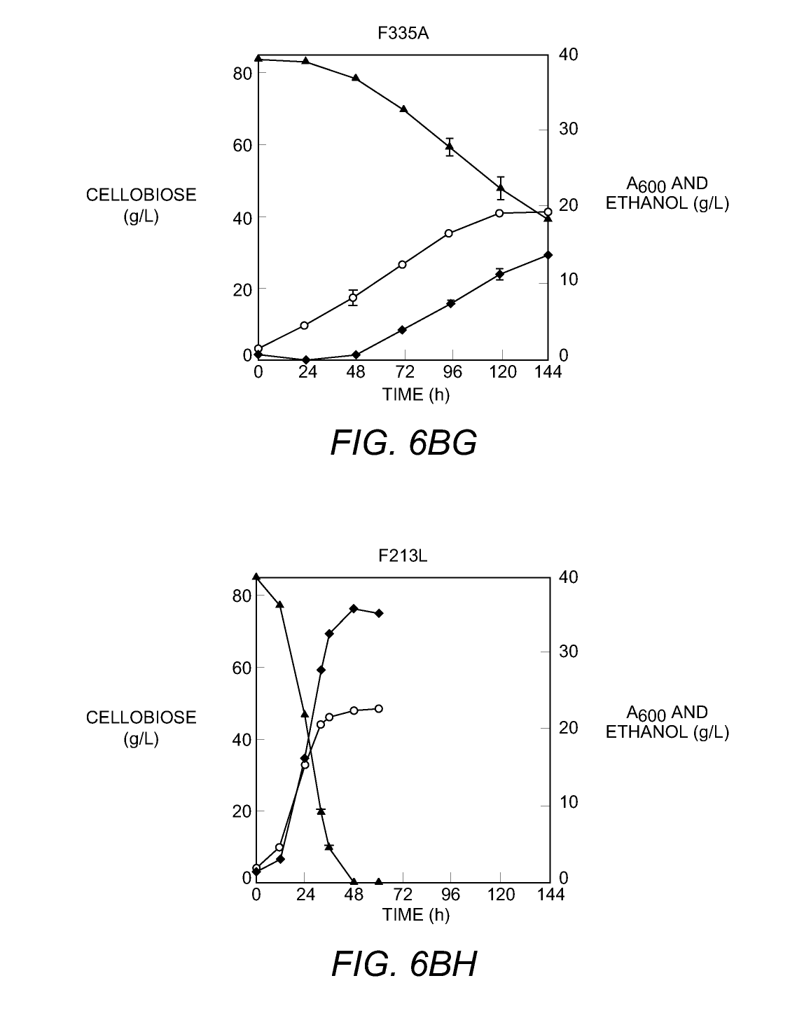

In order to determine whether other CDT-1 mutants have a similar affect on cellobiose fermentation, seven formerly identified alanine mutants were tested. It was previously observed that expression of each these transporter mutants in combination with the β-glucosidase GH1-1 allow S. cerevisiae to grow at different rates on cellobiose. Compared to expression of wild-type CDT-1, expression of the transporter mutants G91A and F335A resulted in faster growth rates, expression of the transporter mutants Q104A and F170A resulted in intermediate growth rates, expression of and the transporter mutants E194A and R174A resulted slow growth rates (FIG. 6A).

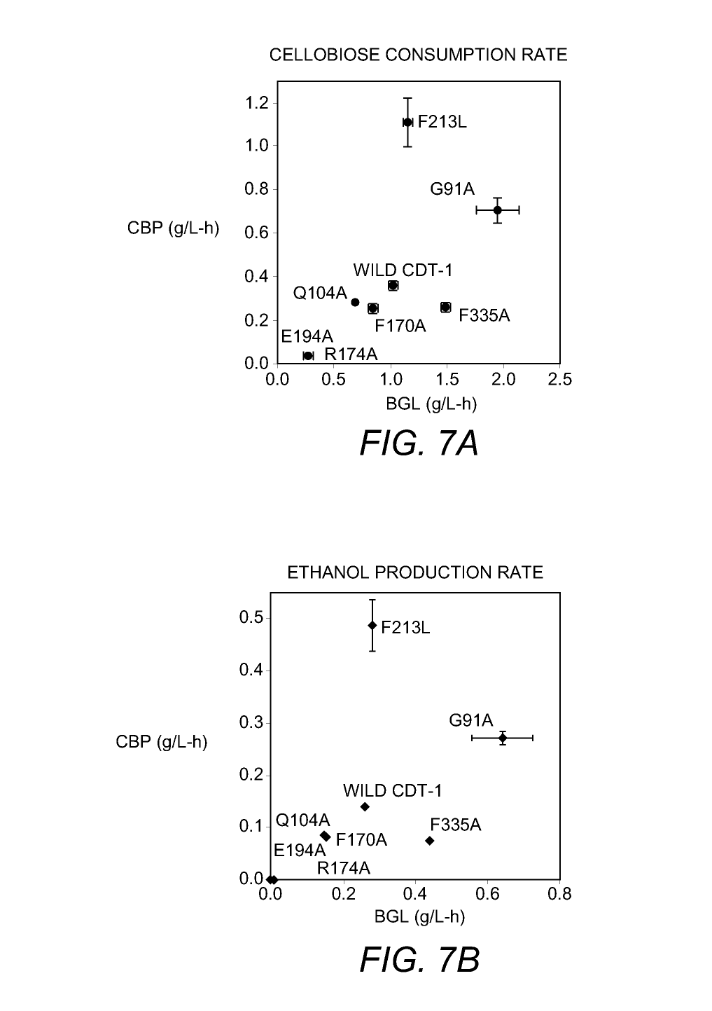

Fermentation rates of yeast strains expressing each of the seven transporters in combination with GH1-1 followed this same trend with G91A>F335A>F213L>WT>F170A>Q104A>R174A>E194A (FIGS. 6A, 7, and Table 10). However, a different trend was observed in strains expressing each of the seven transporters in combination with the cellobiose phosphorylase SdCBP. In this case the trend was F213L>G91A>WT>Q104A>F170A>F335A>R174A>E194A (FIG. 6B). The most notable difference being that the optimal transporter was different between strains expression GH1-1 and SdCBP.

TABLE 10

| Cellobiose consumption rate (g/L-h) | | | Ethanol production rate (g/L-h) | | |
|---|---|---|---|---|---|
| | With BGL | With CBP | | With BGL | With CBP |
| WT CDT-1 | 1.02 ± 0.0600 | 0.36 ± 0.0350 | WT CDT-1 | 0.26 ± 0.011 | 0.2718 ± 0.0100 |
| G91A | 1.95 ± 0.1899 | 0.70 ± 0.0566 | G91A | 0.64 ± 0.0842 | 0.0855 ± 0.0193 |
| Q104A | 0.69 ± 0.0373 | 0.28 ± 0.0037 | Q104A | 0.15 ± 0.0100 | 0.0775 ± 0.0022 |
| F170A | 0.85 ± 0.0730 | 0.26 ± 0.0351 | F170A | 0.16 ± 0.0104 | 0.0000 ± 0.0136 |
| R174A | 0.27 ± 0.0475 | 0.04 ± 0.0028 | R174A | 0.02 ± 0.0020 | 0.0000 ± 0.0000 |
| E194A | 0.27 ± 0.0693 | 0.03 ± 0.0050 | E194A | 0.01 ± 0.0144 | 0.0750 ± 0.0000 |
| F335A | 1.49 ± 0.0520 | 0.26 ± 0.0276 | F335A | 0.44 ± 0.0113 | 0.1400 ± 0.0081 |
| F213L | 1.15 ± 0.0600 | 1.11 ± 0.1110 | F213L | 0.28 ± 0.0100 | 0.4860 ± 0.0490 |

Table 10 quantifies cellobiose consumption and ethanol production of engineered yeast strains with the various cdt-1 mutants. The D452-2 strain of S. cerevisiae was transformed with either WT cdt-1 or one of the cdt-1 mutants, and a codon-optimized cellobiose phosphorylase gene from S. degradans or the β-glucosidase gene gh1-1. The rate at which the strains consumed cellobiose and produced ethanol is shown in Table 10. All values are the means of the results for two independent fermentations, and error bars represent the standard deviations of the results between two fermentations.

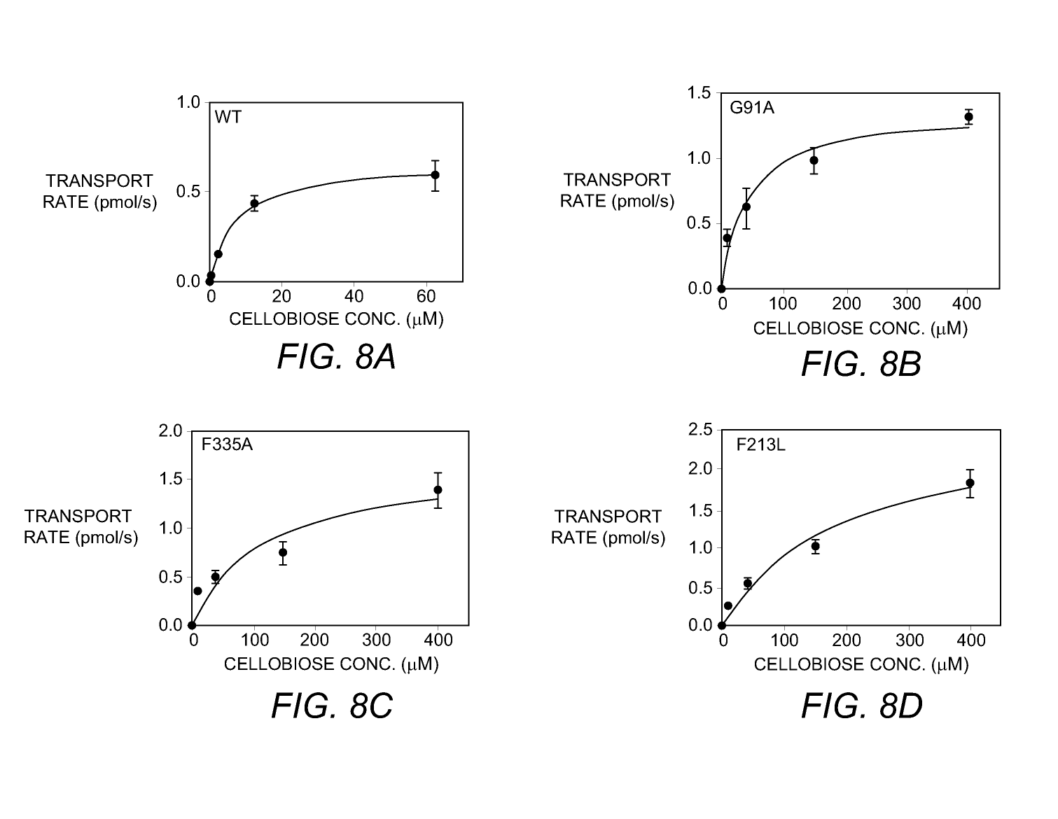

To assess transporter function directly, a Michaelis-Menten analysis of the 4 transporters giving the best fermentation rates (WT, G91A, F335A, and F213L) was performed by measuring the rate of [³H] cellobiose uptake into cells at various concentrations of [³H] cellobiose (FIG. 8). The results are quantified in Table 11. Compared to WT (K_{M}=7.6±1.5 μM, Vₘₐₓ=0.60±0.03 μmol/s), all of the mutants had a lower affinity for cellobiose. However, the three mutants had a higher Vₘₐₓ. Thus, transporters that allow faster fermentation rates also transport cellobiose at a higher maximum velocity.

TABLE 11

| | | Vₘₐₓ | Vₘₐₓ |
|---|---|---|---|
| | | (pmol/s, norm. to | (pmol/s, norm. |
| | K_{M} (μM) | 2 × 10⁸ cells) | for GFP fluo.) |
| WT | 7.6 ± 1.5 | 0.60 ± 0.03 | 0.86 ± 0.05 |
| F213L | 188.8 ± 55.3 | 2.66 ± 0.37 | 2.38 ± 0.31 |
| F335A | 114.8 ± 46.4 | 1.64 ± 0.24 | 1.75 ± 0.26 |
| G91A | 43.5 ± 10.1 | 1.51 ± 0.10 | 1.87 ± 0.12 |

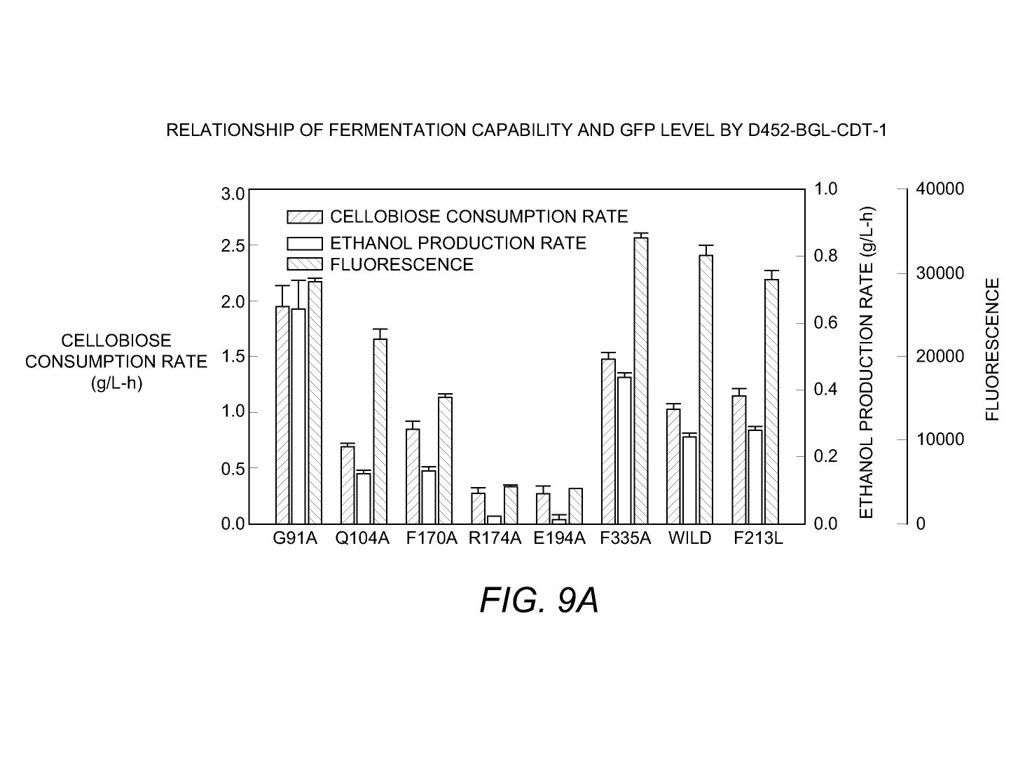

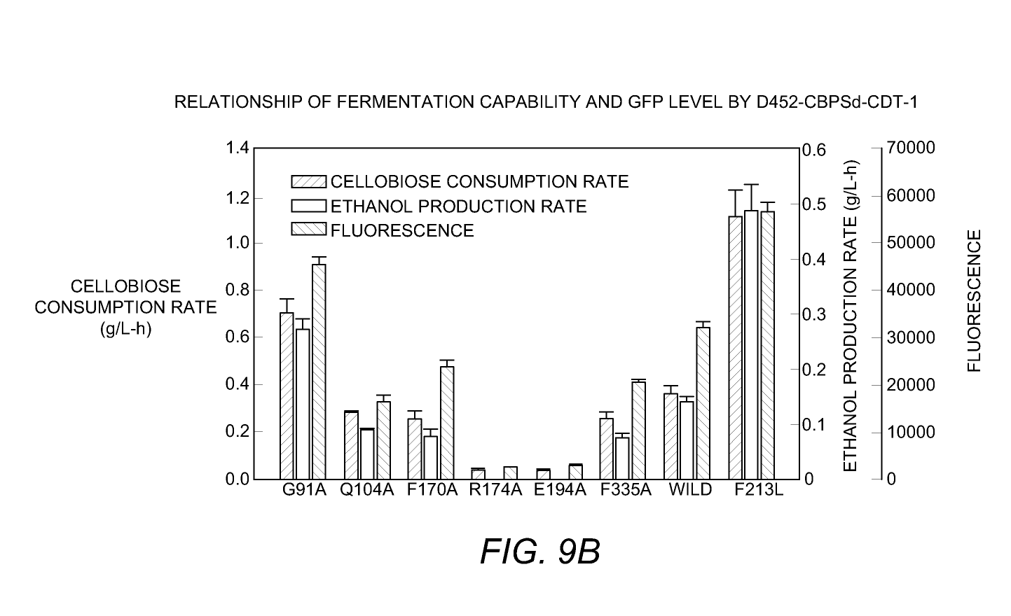

In the above measurements of transporter kinetics, strains were grown on glucose, which resulted in each transporter being expressed at similar levels (±˜20% of WT) as judged by the amount of GFP fluorescence per cell. However, during fermentation of cellobiose this was not the case. There were large differences in the amount of GFP fluorescence per cell depending upon which transporter mutant the yeast cells expressed (FIG. 9). Furthermore, these differences were strongly correlated to cellobiose consumption and ethanol production rates.

Analysis of Intracellular Cellobiose Metabolism

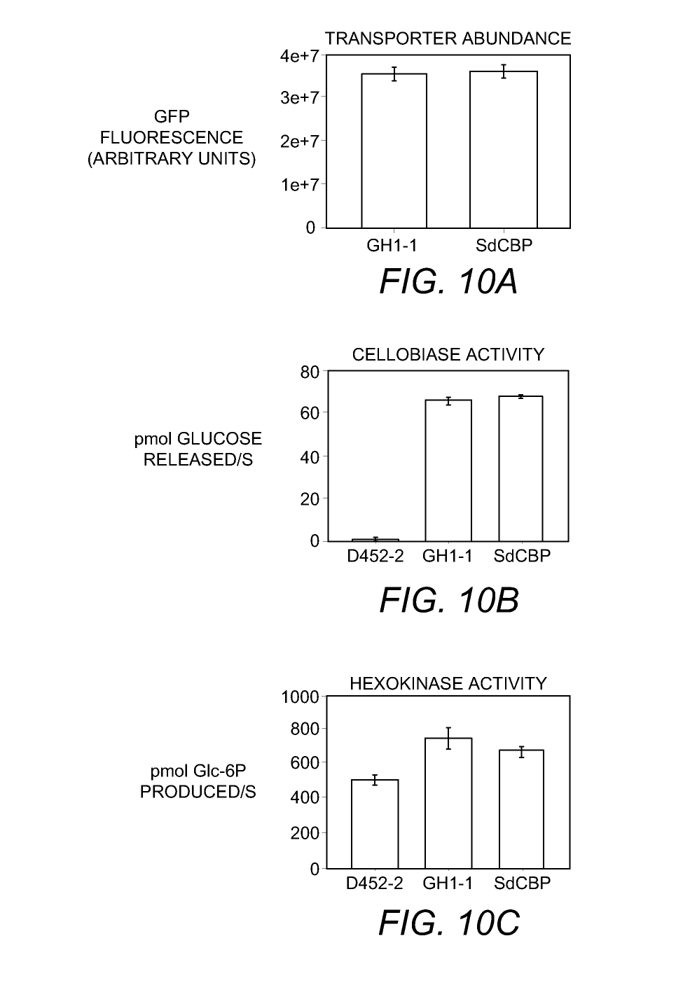

Despite the use of identical transporter genes, the distinct differences in strain performance when cellobiose is cleaved by hydrolysis versus phosphorolysis suggest that the fate of transported cellobiose depends upon the mechanism and kinetics of its intracellular processing. Therefore, the intracellular metabolism of the engineered yeast strains was analyzed by measuring select enzymatic activities in whole cell extracts. The extracts were prepared from cells during the exponential phase of cellobiose fermentation by strains expressing CDT-1 and either GH1-1 or SdCBP. The strains expressed CDT-1 at identical levels according to the amount of GFP fluorescence (FIG. 10A). For additional comparisons, extracts were prepared from WT D452-2 yeast during the exponential phase of glucose fermentation.

To determine if there was a significant difference in the ability of the two strains to cleave intracellular cellobiose, the amount of cellobiase activity in 10 μg of extract (as determined by the Bradford assay) was measured. Cellobiase activity was defined as the rate of glucose produced from cellobiose regardless of the mechanism, and was similar in strains expressing GH1-1 or SdCBP (FIG. 10B). As expected, there was no detectable cellobiase activity in extracts of WT D452-2 (FIG. 10B). It is important to note that this assay underestimates the activity of cellobiose phosphorylase by 50% because the glucose-1-phosphate produced by this enzyme is not accounted for.

Moreover, the amount of hexokinase activity in 10 μg of these extracts was indistinguishable between strains with GH1-1 and SdCBP (FIG. 10C). Additionally, both strains had slightly more hexokinase activity than the WT D452-2 strain (FIG. 10C).

As noted above, during cellobiose fermentation performed with strains expressing CDT-1 mutants with GH1-1, glucose and cellodextrins accumulated in the media. Yet this is not observed during fermentation with strains expressing CDT-1 mutants with SdCBP. The accumulation of glucose and cellodextrins is believed to be due to their efflux following either cellobiose hydrolysis or transglycosylation. Transglycosylation by β-glucosidases is a well documented activity in which high concentrations of glucose and cellobiose are dehydrated to cellotriose and cellotetraose.

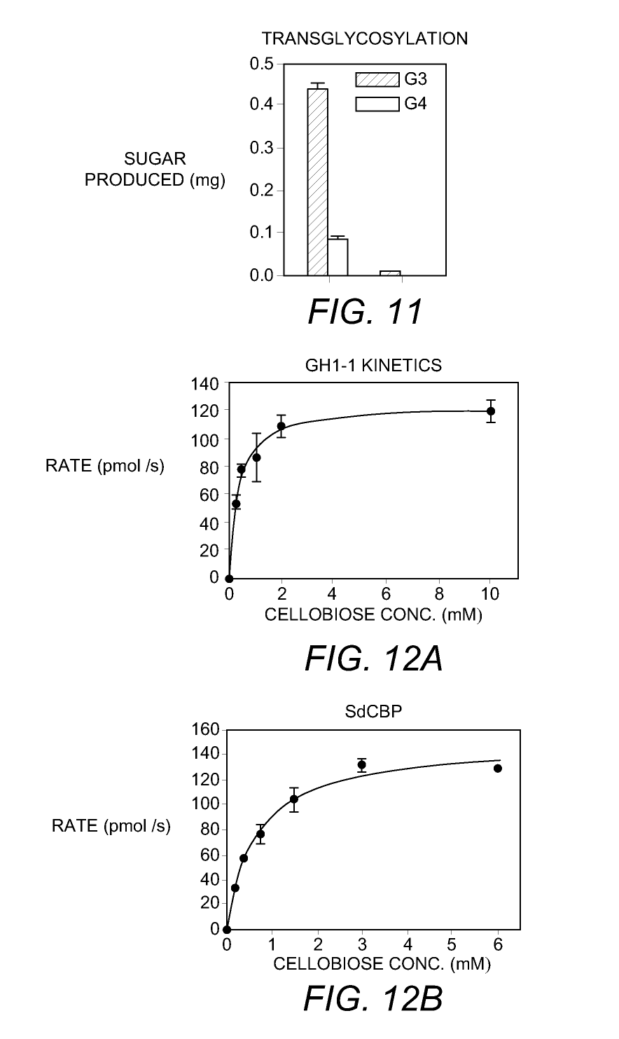

To assay the transglycosylation activity of GH1-1 and SdCBP, both enzymes were enriched from cell extracts by IMAC. The enriched enzymes were then incubated with 20% (w/v) cellobiose for 24 hours, and the reaction products were analyzed by HPLC. GH1-1, but not SdCBP, had significant transglycosylation activity under these conditions producing approximately 0.45 mg of cellotriose and approximately 0.1 mg of cellotetraose from 2 mg of cellobiose (FIG. 11). The kinetic parameters of the enriched proteins were also measured, and the results are depicted in FIG. 12 and quantified in Table 12.

TABLE 12

| | K_{M} (mM) | k_{cat} (s⁻¹) | k_{cat}/K_{M} |
|---|---|---|---|
| GH1-1 | 0.32 | 7 | 21910 |
| SdCBP | 0.65 | 7 | 11515 |

Cellobiose is not naturally found in the cytosol of S. cerevisiae, and its presence may have numerous unforeseen consequences. One potential consequence may involve hexokinases. Hexokinases bind tightly to glucose, and therefore may interact with, or be inhibited by, the high concentration of cellobiose found in the yeast strains engineered to express a cellodextrin transporter and a cellobiose phosphorylase.

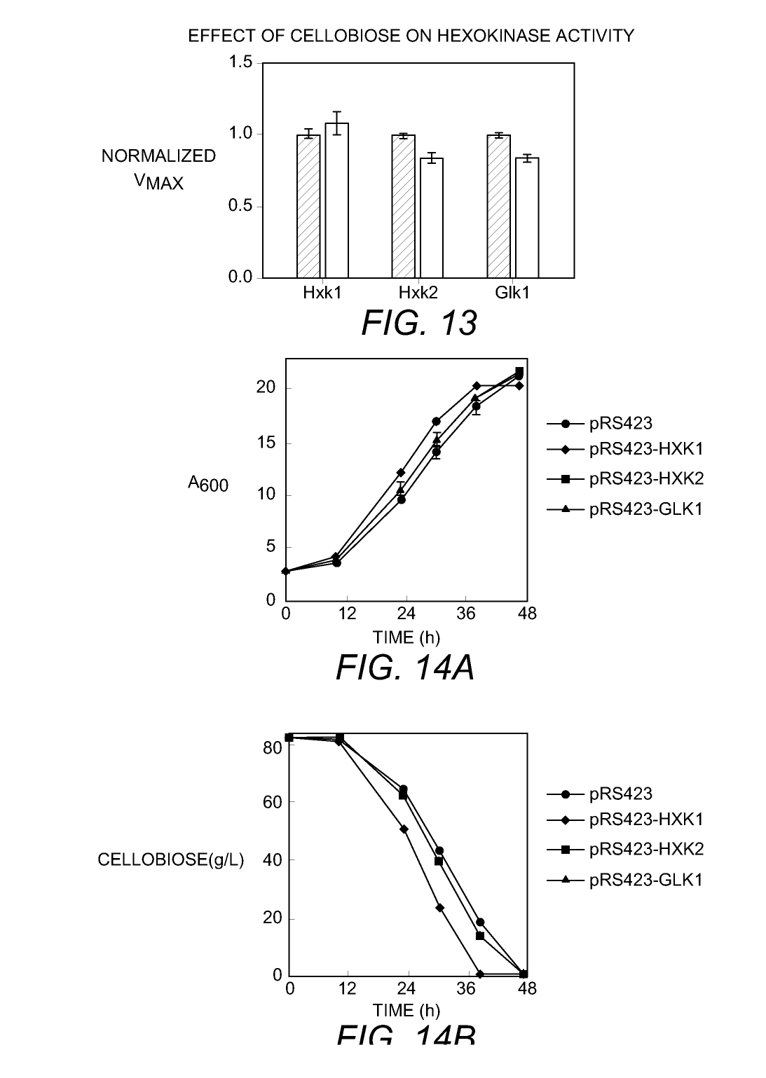

To test whether cellobiose inhibits yeast hexokinases, the hexokinases were expressed and purified from E. coli. The activity of the purified enzymes was then assayed in the presence and absence of up to 184 mM cellobiose. At these extreme concentrations of cellobiose the activity of hexokinase Hxk1 was unaffected (FIG. 13). However, the activity of hexokinases Hxk2 and Glk1 were reduced by ˜20% (FIG. 13).

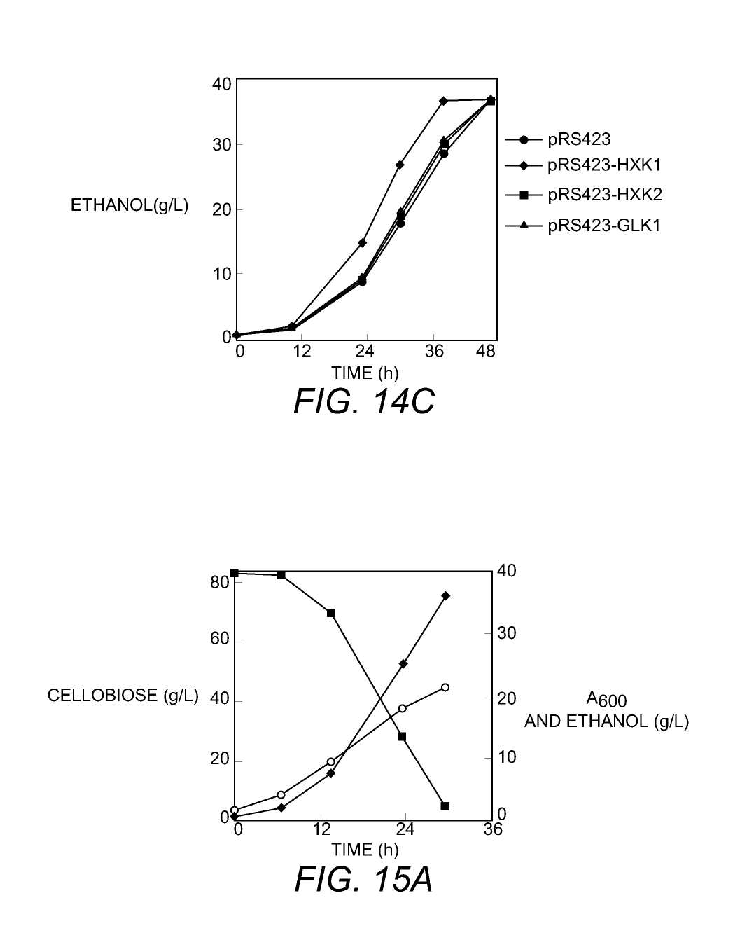

To determine whether or not hexokinase activity was limiting in the engineered pathways, all three yeast hexokinases (HKX1, HXK2, and GLK1) were over-expressed in a strain expressing the mutant cellodextrin transporter CDT-1 (F213L) and the S. degradans cellobiose phosphorylase SdCBP (FIG. 14). While the over-expression of HXK2 or GLK1 did not change strain performance, over-expression of HXK1 resulted in a 28% improvement in ethanol productivity (FIG. 14C and Table 13). Cell density, ethanol production and yield, and ethanol productivity for these strains are summarized in Table 13.

TABLE 13

| | OD | Ethanol | Yield | Productivity |
|---|---|---|---|---|
| | (600 nm) | (g/L) | (g/g) | (g/L-h) |
| pRS423 | 19 | 28 | 0.46 | 0.75 |
| PRS423-HXK1 | 20 | 36 | 0.46 | 0.96 |
| PRS423-HXK2 | 19 | 30 | 0.46 | 0.79 |
| PRS423-GLK1 | 19 | 31 | 0.46 | 0.81 |

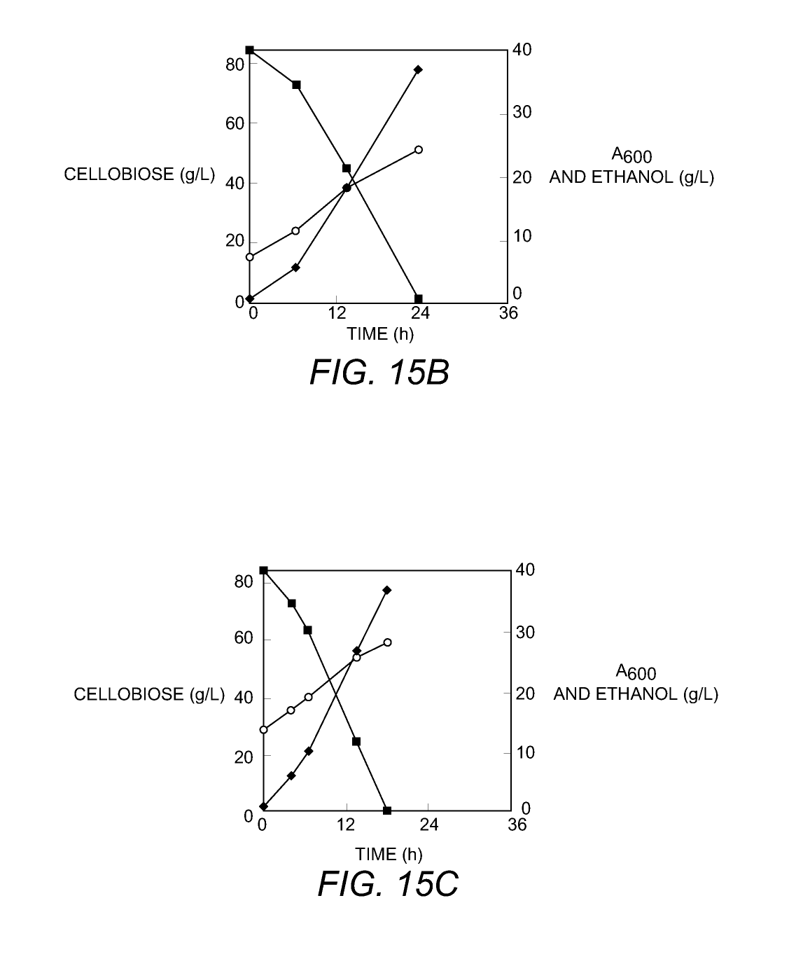

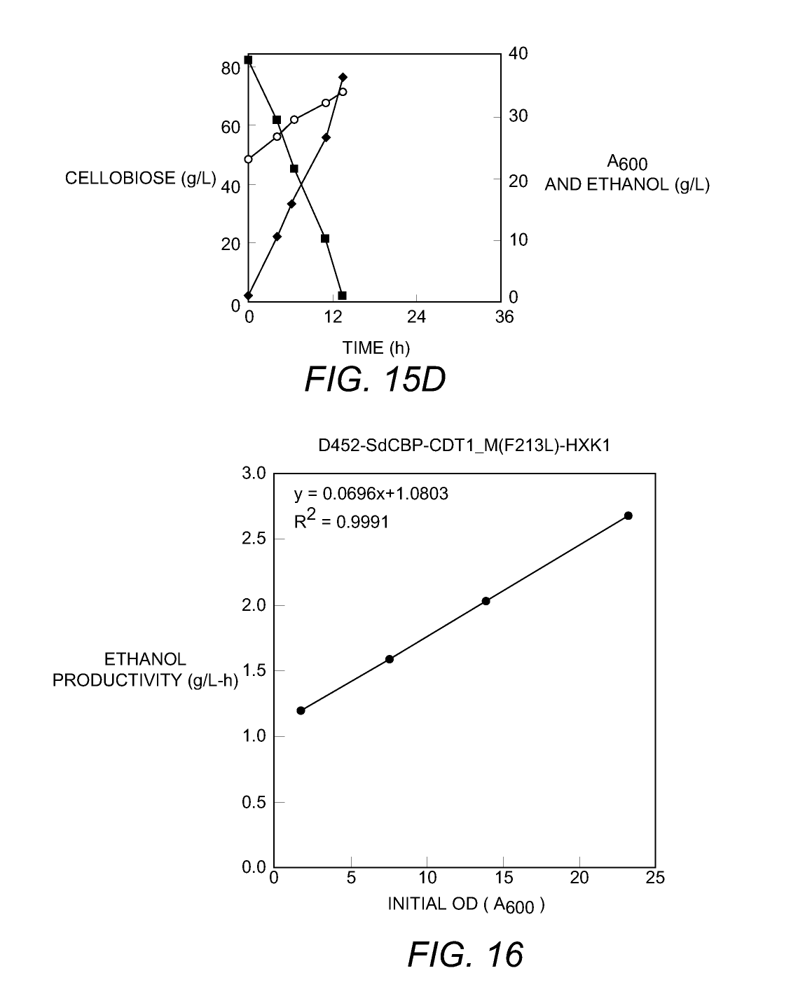

Furthermore, the ethanol productivity of the strain expressing HXK1 increased linearly with increasing initial cell densities, up to 2.69 g ethanol/L-h with an initial density (OD) of 23.1 (FIGS. 15 and 16).

Discussion

Disclosed herein is an analysis of two cellobiose-fermentation pathways engineered into S. cerevisiae with the intention of improving fuel and chemical production from lignocellulosic feedstock. The first pathway utilizes a cellodextrin transporter and an intracellular β-glucosidase, as reported previously, while the second pathway utilizes the same cellodextrin transporter with an intracellular cellobiose phosphorylase. The hydrolytic pathway appears commonly in cellulolytic fungi, where its biological role is believed to include cellulose sensing and metabolism, and the enablement of symbiotic relationships with plants. The phosphorolytic pathway exists within prokaryotes that are simultaneously cellulolytic and anaerobic, where it maximizes energy gain from cellodextrin consumption when respiration is impossible. This is because phosphorolytic cleavage produces glucose-1-P, thus reducing the amount of ATP that must be consumed to phosphorylate cellodextrin-derived glucose before it may enter glycolysis.

Functional cellobiose fermentation pathways were successfully constructed with cellobiose phosphorylases with no more than 71% pairwise amino acid identity from three different anaerobic bacteria, suggesting that special properties besides core enzymatic function are not necessary. However, the above results suggest that it is important to codon-optimize these genes for expression in S. cerevisiae, and that results vary with the optimization algorithm used.

Un-optimized strains with the cellodextrin transporter, CDT-1, and an intracellular cellobiose phosphorylase (CBP) fermented cellobiose to ethanol slower than strains with CDT-1 and an intracellular β-glucosidase. While this would suggest that CBP activity is limiting, other results argue against this. After serial passage of a phosphorolytic strain in cellobiose media, a new strain emerged that fermented cellobiose approximately as fast as strains with the hydrolytic pathway, and the point mutation necessary for this improvement did not affect the CBP gene or its expression as would be expected if CBP activity was limiting. Rather the cellodextrin transporter, CDT-1, was mutated to a low-affinity/high-capacity form. Furthermore, other low-affinity/high-capacity forms of CDT-1 improved fermentation rates with the phosphorolytic pathway. Together these observations suggest that transport rate is actually limiting the phosphorolytic strain.

However, it does not appear that the ideal transporter is simply one with the highest capacity, but rather that the optimal transport kinetics depends upon the metabolic context. This is clearly seen in our comparison of strains with a variety of CDT-1 mutants and either an intracellular beta-glucosidase or cellobiose phosphorylase. This analysis showed that identical transporters did not give identical results in the context of either the hydrolytic or phosphorolytic pathway. For example, the CDT-1 mutant G91A is optimal for the hydrolytic pathway, while the mutant F213L is optimal for the phosphorolytic pathway.

Ultimately the performance of any metabolic pathway may depend on both the maximum capacity of the enzymes in the pathway, and the in vivo activity of the enzymes as dictated by their interactions with small-molecular substrates, products, and effectors. In this study, a limited comparison has been made of these factors between the hydrolytic and phosphorolytic strains during cellobiose fermentation. There were no significant differences in the enzyme capacities of the strains leading from cellobiose to glucose-6-phosphate, the entry point to glycolysis. A notable exception is in the capacity of the hydrolytic pathway to transglycosylate cellobiose to longer cellodextrins such as cellotriose and cellotetraose: GH1-1 is capable of this activity and strains with the hydrolytic pathway cause a significant build-up of longer cellodextrins as they ferment cellobiose to ethanol.

The greatest advantage to the cellulolytic pathway may be that its performance continues to improve as higher cell loadings are used. This is particularly important during fuel production, where high-gravity fermentations are commonly employed to rapidly ferment feedstock, thus making optimal use of capital-intensive fermenter space.

### Example 2

This Example describes the identification of conserved motifs in cellodextrin phosphorylases and the identification of additional cellodextrin phosphorylases.

A first round of PSI-BLAST was run using the Clostridium thermocellum (BAA22081.1), Acidovibrio cellulolyticus (ZP_{—}07328763.1) and Clostridium lentocellum (YP_{—}004310865.1) cellodextrin phosphorylase amino acid sequences as simultaneous inputs. From the result of the first round, all hits annotated as “cellodextrin phosphorylase” were used as simultaneous inputs for a second round of PSI-BLAST.

From the results of the second round, all sequences annotated as “cellodextrin phosphorylase” were used to produce a multiple sequence alignment using T-COFFEE.

This multiple sequence alignment was used as an input for the PRATT server (http://web.expasy.org/pratt/) to identify the highest scoring motif. The motif is shown in PROSITE format: G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 14).

The conserved motif was then used to find cellobiose phosphorylase proteins by using the PROSITE server (http://prosite.expasy.org/scanprosite/). The PROSITE server identified 16 additional cellodextrin phosphorylases. The 16 phosphorylases are listed in Table 4 above.

### Example 3

This Example describes the identification of conserved motifs in cellobiose phosphorylases and the identification of additional cellobiose phosphorylases.

Conserved Motif in Cellobiose Phosphorylase

A first round of PSI-BLAST was run using the Saccharophagus degradans (YP_{—}526792.1), Cellvibrio gilvus (2CQS_A) and Clostridium thermocellum (YP_{—}001036707.1) cellobiose phosphorylase amino acid sequences as simultaneous inputs. From the result of the first round, all hits annotated as “cellobiose phosphorylase” or “cellulose degradation product phosphorylase” were used as simultaneous inputs for a second round of PSI-BLAST.

From the results of the second round, all sequences that score higher than Saccharophagus degradans (YP_{—}526792.1), Cellvibrio gilvus (2CQS_A) and Clostridium thermocellum (YP_{—}001036707.1) cellobiose phosphorylases were used to produce a multiple sequence alignment using EXPRESSO with PDB file, 2CQS, as a structural template.

This multiple sequence alignment was used as an input for the PRATT server (http://web.expasy.org/pratt/) to identify the highest scoring motif. The motif is shown in PROSITE format: Y-Q-[CN]-M-[IV]-T-F-[CN]-[FILMV]-[AS]-R¹-[ST]-[AS]-S-[FY]-[FY]-E-[STV]-G-x-[GS]-R²-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 15). Within this motif, R¹ indicates an arginine involved in inorganic phosphate binding (R351) in the Cellvibrio gilvus cellobiose phosphorylase. R² indicates an arginine involved in forming the cellobiose binding pocket (R362) in the Cellvibrio gilvus cellobiose phosphorylase.

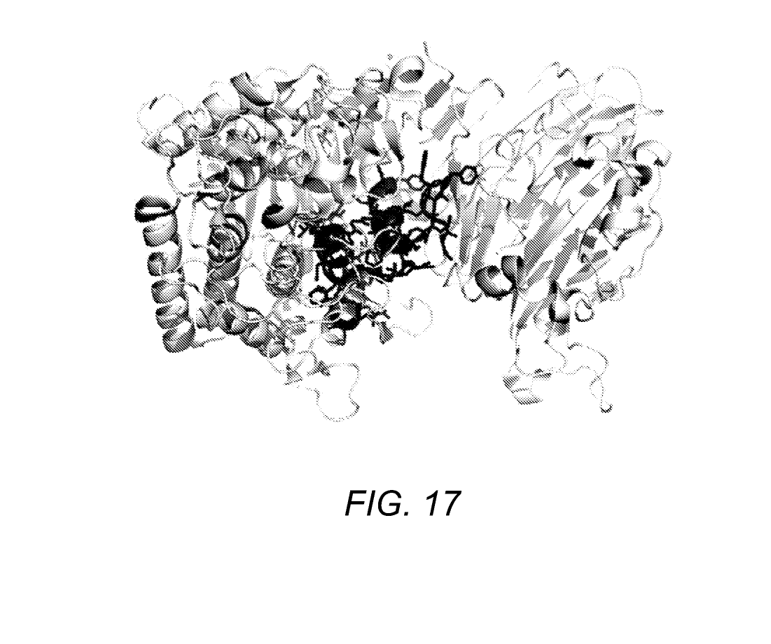

The conserved motif was identified in the crystal structure of the Cellvibrio gilvus cellobiose phosphorylase PDB 2CQS (FIG. 17).

The conserved motif was then used to find cellobiose phosphorylase proteins by using the PROSITE server (http://prosite.expasy.org/scanprosite/). The PROSITE server identified 91 additional cellobiose phosphorylases. The 91 phosphorylases are listed in Table 5 above.

Conserved Motif Found in Both Cellobiose Phosphorylases and Cellodextrin Phosphorylases

Cellodextrin phosphorylases appear to have diverged from cellobiose phosphorylases in how the inorganic phosphate is bound. For example, the two conserved arginines identified in the Cellvibrio gilvus cellobiose phosphorylase are not conserved across cellodextrin phosphorylases. Although no crystal structure of a cellodextrin phosphorylase is known, a similar approach to that described above for the crystal structure of the Cellvibrio gilvus cellobiose phosphorylase was used to identify a PROSITE motif that is conserved among both cellobiose phosphorylases and cellodextrin phosphorylases. The motif is shown in PROSITE format: Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233).

Based on analysis of the Cellvibrio gilvus cellobiose phosphorylase crystal structure with PDB 3QG0, the conserved motif appears to line the substrate-binding site of cellobiose and cellodextrin phosphorylases.

### Example 4

This Example describes enhanced cellodextrin utilization by an engineered Saccharomyces cerevisiae expressing a cellodextrin transporter and a cellodextrin phosphorylase.

Introduction

Three genes (CDP-Acell, CDP-Clent, and CDP-Ctherm) coding for cellodextrin phosphorylase (CDP) were introduced into S. cerevisiae expressing the wild-type cellodextrin transporter CDT-1 or the mutant cellodextrin transporter CDT-1 (F213L). In order to check the capability of utilizing cellobiose, cellotriose or cellotetraose by engineered S. cerevisiae strains, small-scale fermentation experiments were performed in YP medium containing cellobiose, cellotriose, or cellotetraose.

Materials and Methods

Strains

The S. cerevisiae strains that were utilized are described in Table 14.

### Table 14

TABLE 14

| | | Cellodextrin |
|---|---|---|
| Name | CBP/CDP | Transporter |
| D452-2 | None | None |
| D452-SdCBP-CDT-1 | SdCBP | CDT-1 |
| D452-SdCBP-CDT-1_F213L | SdCBP | CDT-1 (F213L) |
| D452-CDP_Acell-CDT-1 | CDP_Acell | CDT-1 |
| D452-CDP_Clent-CDT-1 | CDP_Clent | |
| D452-CDP_Ctherm -CDT-1 | CDP_Ctherm | |
| D452-CDP_Acell-CDT-1_F213L | CDP_Acell | CDT-1 (F213L) |
| D452-CDP_Clent -CDT-1_F213L | CDP_Clent | |
| D452-CDP_Ctherm -CDT-1_F213L | CDP_Ctherm | |

In Table 14, “Name” refers to the strain name; “CBP.CDP” refers to the cellobiose phosphorylase or cellodextrin phosphorylase gene that is expressed by the strain; and “Cellodextrin Transporter” refers to the cellodextrin transporter gene that is expressed by the strain.

Fermentation Conditions

Fermentation experiments were performed in YP medium containing 5 g/L of cellobiose, cellotriose, or cellotetraose. A 96 well plate was used to monitor growth on cellodextrin as a sole carbon source. The culture volume in each well was 200 μL and 50 μL of mineral oil was overlaid on the top of the culture to prevent evaporation of the medium during growth measurement. The initial cell density was adjusted to OD600=˜0.2. A Synergy H4 hybrid Microplate Reader (BioTek Instruments Inc., Winooski, Vt.) was used for measuring absorbance at 600 nm with a continuous mixing option.

Results

Cellobiose or Cellodextrin Utilization by S. cerevisiae Strains Expressing the Wild-Type CDT-1 Gene and Various Cellodextrin Phosphorylase (CDP) Genes

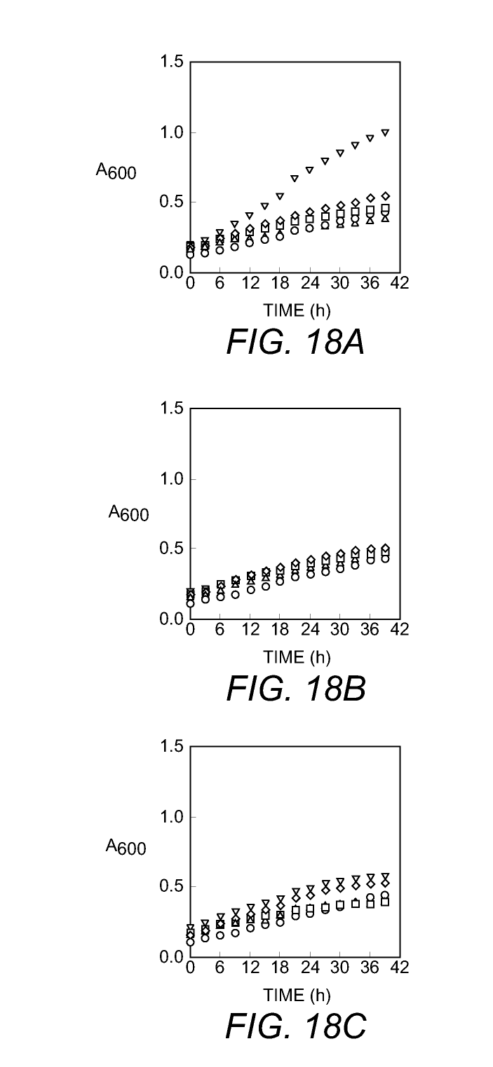

Each of three cellodextrin phosphorylases (CDP_Clent, CDP_Ctherm, and CDP_Acell) from Clostridium lentocellum, Clostridium thermocellum, Acidovibrio cellulolyticus, respectively; and cellodextrin transporter (cdt-1) from Neurospora crassa were introduced in S. cerevisiae D452-2. The resulting transformants were subjected to growth tests on cellobiose, cellotriose, and cellotetraose (FIG. 18). As a control strain for growth comparison, an engineered strain expressing cellobiose phosphorylase (SdCBP) and cellodextrin transporter (CDT-1) was used. In cellobiose medium, only the D452-SdCBP-CDT-1 strain harboring cellobiose phosphorylase (SdCBP) and cellodextrin transporter (CDT-1) was able to grow well. While engineered S. cerevisiae strains having cellodextrin phosphorylase and cellodextrin transporter did not grow on cellobiose, the D452-CDP_Clent-CDT-1 strain expressing cellodextrin phosphorylase (CDP_Clent) from C. lentocellum and cellodextrin transporter (CDT-1) was able to grow on cellobiose very slowly (FIG. 18A). This result suggests that CDP_Clent can use cellobiose as a substrate. When cellotriose was used as a carbon source, none of the engineered strains showed measurable growth. Only the D452-CDP_Clent-CDT-1 strain grew very slowly (FIG. 18B). When cellotetraose was used as a sole carbon source, the D452-SdCBP-CDT-1 and D452-CDP_Clent-CDT-1 strain showed measurable growth (FIG. 18C).

These results suggest that the cellodextrin phosphorylase CDP_Clent from C. lentocellum can facilitate utilization of cellobiose, cellotriose, and cellotetraose when cellodextrin transporter (CDT-1) is co-expressed in S. cerevisiae.

Enhanced Cellobiose or Cellodextrin Utilization by S. cerevisiae Strains Expressing the Mutant CDT-1 F213L Gene and Various CDP Genes

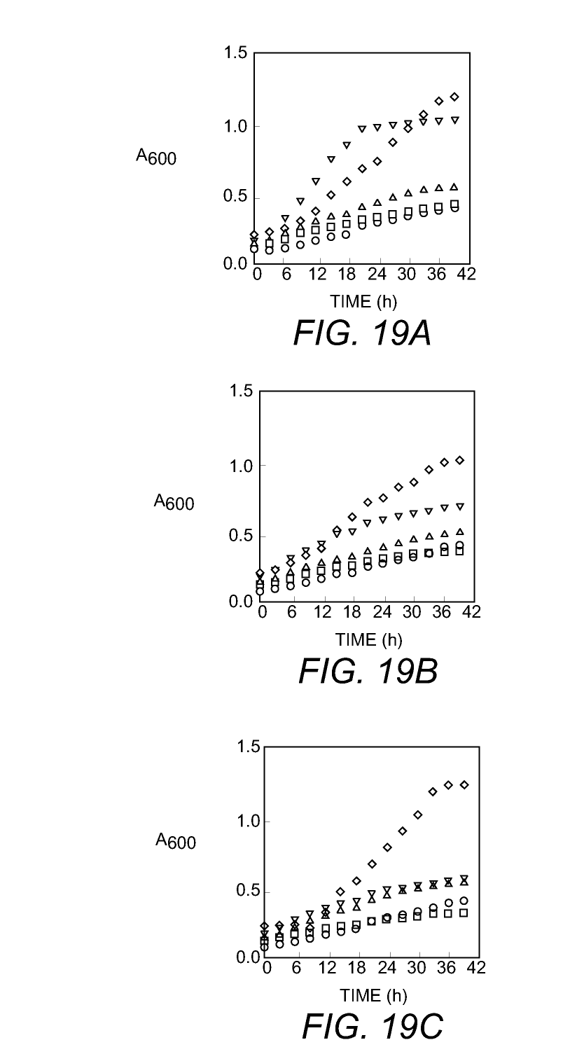

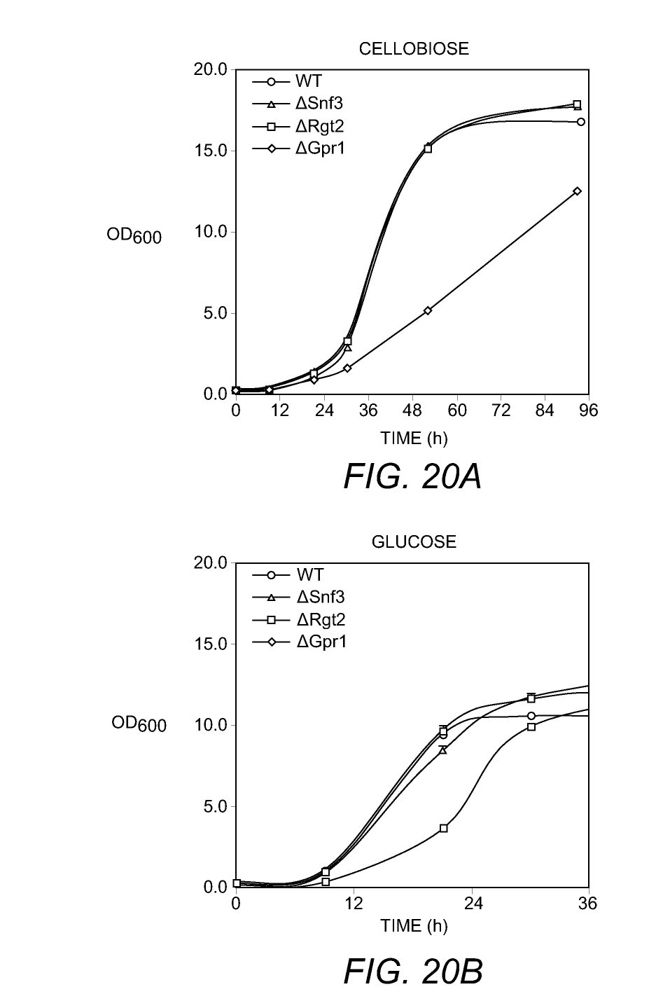

Utilization of cellodextrin by the engineered S. cerevisiae expressing various cellodextrin phosphorylases can be limited by the capacity of the cellodextrin transporter (CDT-1). Therefore, a new set of engineered strains was constructed that expressed each of three cellodextrin phosphorylases (CDP_Acell, CDP_Clent, and CDP_Ctherm) and a mutant cellodextrin transporter (CDT-1 F213L). In order to compare cellodextrin utilization rates by these strains, cell growth on cellobiose, cellotriose, and cellotetraose was monitored. As a control strain for growth comparison, an engineered strain expressing cellobiose phosphorylase (SdCBP) and cellodextrin transporter (CDT-1 F213L) was used. When cellobiose was used as a carbon source, the D452-SdCBP-CDT-1_F213L strain harboring cellobiose phosphorylase (SdCBP) and a mutant cellodextrin transporter (CDT-1_F213L) grew rapidly as previously observed (FIG. 19A). The D452-CDP_Clent-CDT-1_F213L strain expressing cellodextrin phosphorylase (CDP_Clent) from Clostridium lentocellum and a mutant cellodextrin transporter (CDT-1 F213L) also grew well on cellobiose (FIG. 19A). When cellotriose was used as a carbon source, both the D452-SdCBP-CDT-1_F213L and CDP_Clent-CDT-1_F213L strains were able to grow (FIG. 19B). However, the CDP_Clent-CDT-1_F213L strain grew much better than D452-SdCBP-CDT-1_F213L in contrast to the cellobiose condition. Moreover, the CDP_Clent-CDT-1_F213L strain grew very well even on cellotetraose, while the D452-SdCBP-CDT-1_F213L strain did not show measurable growth on cellotetraose (FIG. 19C). These results suggest that the cellodextrin phosphorylase from Clostridium lentocellum (CDP_Clent) can utilize three tested cellodextrins (cellobiose, cellotriose, and cellotetraose).

The D452-CDP_Ctherm-CDT-1_F213L strain harboring the cellodextrin phosphorylase from Clostridium thermocellum (CDP_Ctherm) and a mutant cellodextrin transporter (CDT-1 F213L) showed measurable growth on cellotriose and cellotetraose (FIGS. 19B and 19C). However, the D452-CDP_Acell-CDT-1_F213L strain expressing the cellodextrin phosphorylase from Acidovibrio cellulolyticus (CDP_Acell) and a mutant cellodextrin transporter (CDT-1 F213L) did not show any measureable growth on cellobiose, cellotriose, or cellotetraose.

Improved cellodextrin utilization by engineered S. cerevisiae was observed when a mutant cellodextrin transporter (CDT-1 F213L) was paired with either a cellobiose phosphorylase or cellodextrin phosphorylase, suggesting that the CDT-1 F213L transporter is capable of facilitating efficient transportation of cellodextrins, as well as cellobiose, with a higher degree of polymerization (DP).

### Example 5

This Example describes cellobiose-mediated growth rates of S. cerevisiae strains that were transformed with the cellodextrin transporter CDT-1 and various Sacharophagus degradans cellobiose phosphorylase mutants (SdCBP).

Materials and Methods

Plasmids each encoding Sacharophagus degradans cellobiose phosphorylase (SdCBP) containing point mutations were transformed into the S. cerevisiae D452-2 strain that expresses the CDT-1 cellodextrin transporter. For each CBP mutant a single colony was picked to inoculate 5 mL starter cultures in YP media containing 2% cellobiose as the carbon source.

The initial OD for fermentation cultures was set to −0.5 and fermentation experiments were carried out using 5 mL of YP media with 8% cellobiose as the carbon source, in 50 mL Falcon tubes. Cultures were grown at 30° C. with continuous shaking. Fermentation experiments were also performed on S. cerevisiae D452-2 strains containing the wild-type SdCBP or the wild-type β-glucosidase GH1-1.

The OD600 of each culture was measured every 12 hours and growth rates (increase in growth per hour) are shown in Table 15. Each point represents the average of three measurements.

Results

The results depicted in Table 15 show that strains containing certain CBP mutants had growth rates that were comparable or greater than that of strains containing the wild-type CBP. In particular the I409M CBP mutant showed had a growth rate of 1.061 hr⁻¹, which was comparable to the growth rate of the wild-type CBP (1.135 hr⁻¹), while the CBP mutant N482D had a growth rate of 1.236 hr⁻¹.

TABLE 15

| | Growth rate | | Growth rate |
|---|---|---|---|
| SdCBP mutant | (hr⁻¹) | SdCBP mutant | (hr⁻¹) |
| Wild-type CBP | 1.135 | Wild-type BGL | 1.3 |
| C484S | 0.696 | I409R | 0.637 |
| E646A | 0.027 | D361A | 0 |
| E693A | 0.184 | H653A | 0.879 |
| N647Q | 0 | I409M | 1.061 |
| F651W | 0.511 | C484A | 0.408 |
| D483A | 0 | R360A | 0.264 |
| D483N | 0 | N482T | 0.376 |
| H653N | 0.759 | N482D | 1.236 |
| K645R | 0 | W481A | 0 |
| Q165A | 0 | Y640W | 0.184 |
| I409Q | 0.872 | | |

### Example 6

This Example describes modifying the glucose response pathway in cellodextrin-transporting cells to optimize metabolism.

In this example, the glucose response pathway of yeast expressing a cellodextrin transporter and an intracellular β-glucosidase is modified to optimize the cell's metabolism of glucose. Yeast strains engineered to express cdt-1 or cdt-2 along with an intracellular β-glucosidase (gh1-1) are further genetically modified to express constitutively active alleles of various glucose response genes. The constitutively-active alleles are expressed under the control of an inducible promoter. Alternatively, the wild-type allele is replaced with the constitutively active allele by targeted recombination.

Yeast are modified with the following mutant alleles by either inducible expression or targeted recombination: the Snf3 R229K allele (Ozcan, PNAS, 1996), the Rgt2 R231K allele (Ozcan, PNAS, 1996), the Yck1-Rgt2tail chimera (Moriya and Johnston, PNAS, 2004), Gpa2^{val132} (Tamaki, J of Biosciences and Bioengineering, 2007), Gpa2^{Q300L} (Wang et al., PLOS Biology, 2004), Ras2^{G19V} (Wang et al., PLOS Biology, 2004), Hxk2^{S14A} (Moreno and Herrero, FEMS, 2002), Pfk27ΔN, and each of the Hxt glucose transporter genes containing the C-terminal tail of Snf3 or Rgt2.

After modification with the mutant glucose response gene alleles, yeast are grown with cellodextrins as the sole carbon source under fermentation conditions. The amount of ethanol produced by each strain is compared to the amount of ethanol produced by a control strain, a yeast strain expressing cdt-1 or cdt-2 and gh1-1 with all wild-type glucose response genes. An increase in ethanol production in an experimental strain compared to the control strain shows that glucose metabolism has been optimized.

### Example 7

This Example describes the results of modifying glucose response pathways in yeast strains that have been engineered to utilize cellobiose.

Introduction

Based on systematically studying cell wall degradation by the cellulolytic fungus Neurospora crassa, two cellodextrin transporter families were discovered. Ethanologenic Sacchromyces cerevisiae heterologously expressing these transporters and intracellular beta-glucosidase from N. crassa can use cellodextrins as substrates to produce ethanol. The identified transporter pathway has opened up a new way of thinking about microbial fermentation of hexoses, as well as of pentoses derived from plant cell walls [1]. However, when non-native cellodextrin substrates are used instead of native substrates, such as glucose, are used, the lag phase in S. cerevisiae is prolonged and the maximal growth rate is lower. This phenomenon is also exemplified by pentose utilization in S. cerevisiae [2]. It seems that S. cerevisiae cannot sense and metabolize these normative substrates as a rapidly fermentable carbon source like glucose [3].

S. cerevisiae uses at least three different pathways to sense glucose in its environment [4]. These include three extracellular pathways (via the G-protein receptor Gpr1, and two transceptors Snf3 and Rgt2), and one intracellular pathway that involves Ras/PKA. Since the cellobiose transport pathway would likely bypass Gpr1, Snf3 or Rgt2, it may only be capable of partially activating the Ras/PKA pathway following transport and hydrolysis of cellobiose. Thus, the metabolic state of S. cerevisiae growing on cellobiose is not optimized to handle the flux of glucose that cellobiose hydrolysis provides. In order to address the above hypothesis, key genes in the signaling pathway (Table 16) were mutated, either by deletion or constitutive activation, to probe the length of the lag phase and growth rates of the resulting strains.

TABLE 16

| | |
|---|---|
| Mutant strain of key genes | Origin |

Glucose sensing pathway

| | |
|---|---|
| Snf3 deletion | Open Biosystems |
| Rgt2 deletion | Open Biosystems |
| Gpr1 deletion | Open Biosystems |
| Snf3, Rgt2 double deletion | This study |
| Snf3, Rgt2, Gpr1 triple deletion | This study |
| Rgt1 deletion | Rine lab |

Intracellular signaling pathway

| | |
|---|---|
| Ras2 deletion | Open Biosystems |
| Ras2^{G19V}, constitutively active allele | This study |
| Ras2^{Q77K}, RAS2^{D112Y}, activity reduced alleles | This study |
| Gpa2 deletion | Open Biosystems |
| Gpa2^{G132V}, GPA2^{Q300L}, constitutively active alleles | This study |
| Sch9 deletion | This study |
| Sch9^{3E}, SCH9^{2D3E} | This study |
| Yak1 deletion | Open Biosystems |
| Grr1 deletion | Rine lab |

Glucose repression pathway

| | |
|---|---|
| Hxk2 deletion | Open Biosystems |
| Hxk2wrf without regulatory function strain | This study |
| (GFP-tagged) | |
| Snf1 deletion | Rine lab |
| Mig1 deletion | Rine lab |

Other key genes related to cell growth

| | |
|---|---|
| Rim15 deletion | Open Biosystems |
| Stb3 deletion | Open Biosystems |
| Kcs1 deletion | Open Biosystems |
| Tps1 deletion | Rine lab |

Materials and Methods

Strains and Plasmid Constructs

S. cerevisiae BY4742 (MATalpha his3Δ1 leu2Δ0 met15Δ0 ura3Δ0) were used for engineering of cellobiose metabolism in yeast. Yeast MATalpha knockout strains were obtained from Open Biosystems. Escherichia coli Top10 was used for gene cloning and manipulation. Cellobiose metabolism was composed of Neurospora crassa beta-glucosidase gene (gh1-1) and cellodextrin transporter gene (cdt-1). As previously reported [5], gh1-1 was cloned into the pRS425 plasmid under PGK promoter and CYC1 terminator, and cdt-1 was cloned into the pRS426 under PGK promoter and CYC1 terminator as well as with C-terminal GFP tag.

Medium and Culture Conditions

E. coli was grown in Luria-Bertani medium; 50 g/ml of carbenicillin was added to the medium when required. Yeast strains were cultivated at 30° C. in YP medium (10 g/L yeast extract and 20 g/L Bacto peptone) with 20 g/L of glucose. Transformed strains were grown in the appropriate complete minimal dropout media, supplemented to 100 mg/L adenine hemisulfate. For carbon source transfer experiment, yeast synthetic complete (YSC) medium were used, which contained 6.7 g/L of yeast nitrogen base plus 20 g/L of glucose or 20 g/L cellobiose, and CSM-Leu-Ura which supplied appropriate nucleotides and amino acids.

Mutant Generation

The mutants of Ras2, Gpa2, and Sch9 were generated via site-directed mutagenesis using QuikChange site-directed mutagenesis kit (Stratagene), and confirmed by sequencing. The Hxk2 mutant lacking regulatory function was constructed according to the reported method [6]. The truncation mutant was obtained by PCR-deletion of 30 bp, between nucleotides +19 and +48, the resulting gene HXK2ΔK6M15, expressed a truncated Hxk2 protein without the amino acids from Lys6 to Met15 (Hxk2wrf). Mutant versions of Hxk2, Ras2, Gpa2, and Sch9 were introduced into chromosome to replace the wild type alleles using a two-step (selection, counter-selection) gene replacement method [7]. The wild type allele (ATG to stop codon) was replaced with the URA3 marker using PCR-based gene manipulation methods [8]. The transformants were selected on YSC-Ura plates and verified by PCR with forward primers ˜200 bp upstream of the replaced ORF and reverse primers within the URA3 marker sequence (Table 16). The URA3 marker was then replaced with mutant allele using an identical approach (except selection of positive transformants was carried out on 5-fluoroorotic acid (5-FOA) and verified by PCR with the same forward primers for deletion verification and reverse primers within the replaced ORF (Table 17). Specially, C-terminal GFP tagged Hxk2 and Hxk2wrf were used in gene replacement. Precise gene replacement was confirmed by sequencing.

Table 17 lists the primers used for generating mutants. In Table 16, underlined nucleotides are those that were changed by mutagenesis.

TABLE 17

| | |
|---|---|
| Name | Sequence |

Deletion cassette with URA3 maker gene

| | | |
|---|---|---|
| RAS2-DF | TAACCGTTTTCGAATTGAAAGGAGATATACAGAAAAAAAAGATTCGGTAATCTCCGAACA | (SEQ ID NO: 234) |
| RAS2-DR | TTCTTTTCGTCTTAGCGTTTCTACAACTATTTCCTTTTTACACACCGCATAGGGTAATAA | (SEQ ID NO: 235) |
| GPA2-DF | TTGTTACAGCACAAATCACGCGTATTTTCAAGCAAATATCGATTCGGTAATCTCCGAACA | (SEQ ID NO: 236) |
| GPA2-DR | AGAAGAGGCATGCAGTTTTGTCTCTGTTTTAGCTGTGCATCACACCGCATAGGGTAATAA | (SEQ ID NO: 237) |
| SCH9-DF | ATACTCGTATAAGCAAGAAATAAAGATACGAATATACAATGATTCGGTAATCTCCGAACA | (SEQ ID NO: 238) |
| SCH9-DR | AAGGAAAAGAAGAGGAAGGGCAAGAGGAGCGATTGAGAAACACACCGCATAGGGTAATAA | (SEQ ID NO: 239) |
| HXK2-DF | TATAATTCTCCACACATAATAAGTACGCTAATTAAATAAAGATTCGGTAATCTCCGAACA | (SEQ ID NO: 240) |
| HXK2-DR | GGCACCTTCTTGTTGTTCAAACTTAATTTACAAATTAAGTCACACCGCATAGGGTAATAA | (SEQ ID NO: 241) |

Verification of deletion strain using URA3 maker gene

| | | |
|---|---|---|
| RAS2-UP | CTGGAGCGTGACATTTAGGA | (SEQ ID NO: 242) |
| GPA2-UP | TTGAAGACCCACCGTAACCA | (SEQ ID NO: 243) |
| HXK2-UP | TGATTGCGAGATCCACGAAA | (SEQ ID NO: 244) |
| URA3-R | CCACATCATCCACGGTTCTAT | (SEQ ID NO: 245) |

Mutant gene generation

| | | |
|---|---|---|
| RAS2-MR | ACCAACACCAACACCACCAAC | (SEQ ID NO: 246) |
| RAS2-MF | GTTGGTGGTGTTGGTGTTGGT | (SEQ ID NO: 247) |
| RAS2-77MF | CTATGAGGGAAAAATACATGCGC | (SEQ ID NO: 248) |
| RAS2-77MR | GCGCATGTATTTTTCCCTCATAG | (SEQ ID NO: 249) |
| RAS2-112MF | ATTGAGAGTCAAATATACCGACTATGT | (SEQ ID NO: 250) |
| RAS2-112MR | ACATAGTCGGTATATTTGACTCTCAAT | (SEQ ID NO: 251) |
| GPA2-132MR | ACCACTTTCAACGGCACCCAG | (SEQ ID NO: 252) |
| GPA2-132MF | CTGGGTGCCGTTGAAAGTGGT | (SEQ ID NO: 253) |
| GPA2-300MR | TTCGGAACGCAGTCCACCCAC | (SEQ ID NO: 254) |
| GPA2-300MF | GTGGGTGGACTGCGTTCCGAA | (SEQ ID NO: 255) |
| SCH9-737F | TGCTGGTTTCGAGTTTGTTGATGAGTCCGCCATCG | (SEQ ID NO: 256) |
| SCH9-737R | CATCAACAAACTCGAAACCAGCAAACTTTGCTTGC | (SEQ ID NO: 257) |
| SCH9-758F | CCTACAAAACGAGTACTTTATGGAACCTGGTTCC | (SEQ ID NO: 258) |
| SCH9-758R | CCATAAAGTACTCGTTTTGTAGGAATTTTCTGTTG | (SEQ ID NO: 259) |
| N-SCH9-765F | GGAACCTGGTGAGTTTATCCCGGGAAATCCAAAC | (SEQ ID NO: 260) |
| N-SCH9-765R | CCGGGATAAACTCACCAGGTTCCATAAAGTACTCG | (SEQ ID NO: 261) |
| SCH9-2D-F | GATGACTGCTGACCCGCTAGATCCAGCCATGCAAGCAAAGTTTG | (SEQ ID NO: 262) |
| SCH9-2D-R | GCATGGCTGGATCTAGCGGGTCAGCAGTCATCATCGGCTGGTGC | (SEQ ID NO: 263) |

Verification of gene replacement

| | | |
|---|---|---|
| RAS2-M | ACCGTATTAGCGCTTTGAGC | (SEQ ID NO: 264) |
| HXK2-M-R | TTCAAAGCATTGGCAGCCTT | (SEQ ID NO: 265) |

Carbon Source Transfer Experiment

Recombinant strains containing the cellobiose utilization pathway were grown on YSC plus glucose medium at 30° C. for 64 h to the late stationary phase, then inoculumed to YSC plus glucose and YSC plus cellobiose in parallel. Initial OD₆₀₀ was 0.2. Biological triplicates of all strains were performed in 50-ml tubes containing 10 ml media.

Results and Discussion

Extracellular Cellobiose Sensing by Glucose Sensors and Receptors

One way the yeast S. cerevisiae senses glucose is through two transmembrane glucose sensors, Snf3 and Rgt2. Extracellular glucose causes these sensors to generate an intracellular signal that induces expression of several HXT genes encoding hexose transporters [9]. The glucose signal induces this expression by influencing the function of the Rgt1 transcriptional repressor. Snf3 and Rgt2 have different affinities for glucose and separate, non-redundant functions [10]. Snf3 appears to be a sensor of low levels of glucose whereas Rgt2 is a sensor of high glucose concentrations. In addition to glucose, Snf3 also senses fructose and mannose, as well as the glucose analogues 2-deoxyglucose, 3-O-methylglucoside and 6-deoxyglucose. Transcriptomic analysis of engineered xylose utilizing yeast suggested that extracellular xylose could be sensed by Rgt2 and Snf3 [2].

Another way the yeast S. cerevisiae senses glucose is through Gpr1, a plasma membrane protein with seven transmembrane domains, coupled to the G protein Gpa2 and involved in the increase in cAMP levels triggered by glucose that initiates a signaling cascade leading to stimulation of fermentation [11]. The Gpr1-Gpa2 couple is responsive to glucose and to sucrose but not to other sugars such as fructose, 2-deoxyglucose or xylose; mannose acts as an antagonist, in which 100 mM sugar was used (Rolland et al., 2000). Higher concentration of xylose than 210 mM might be needed to be sensed by Gpr1 [2].

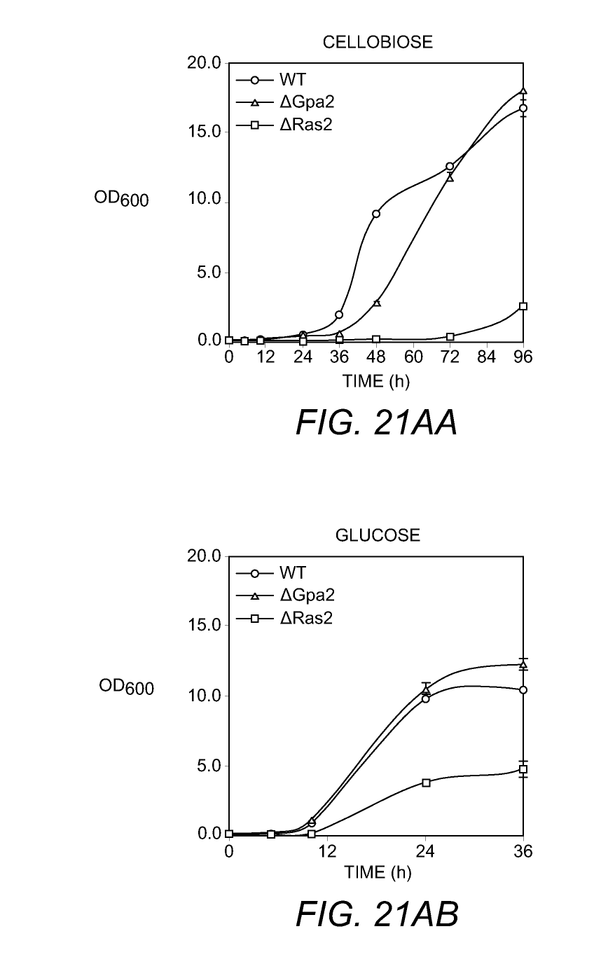

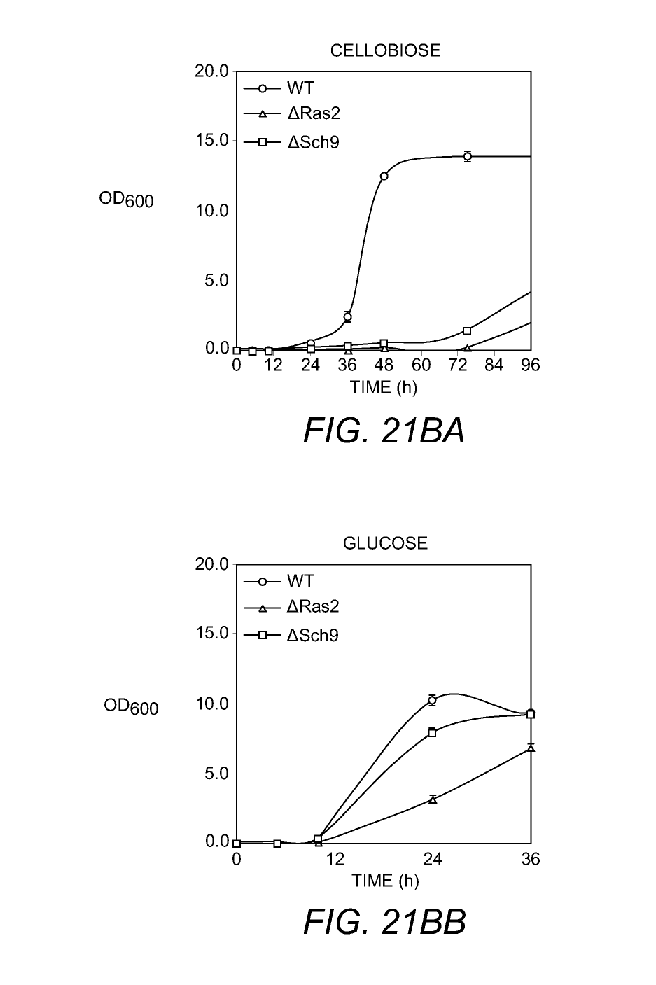

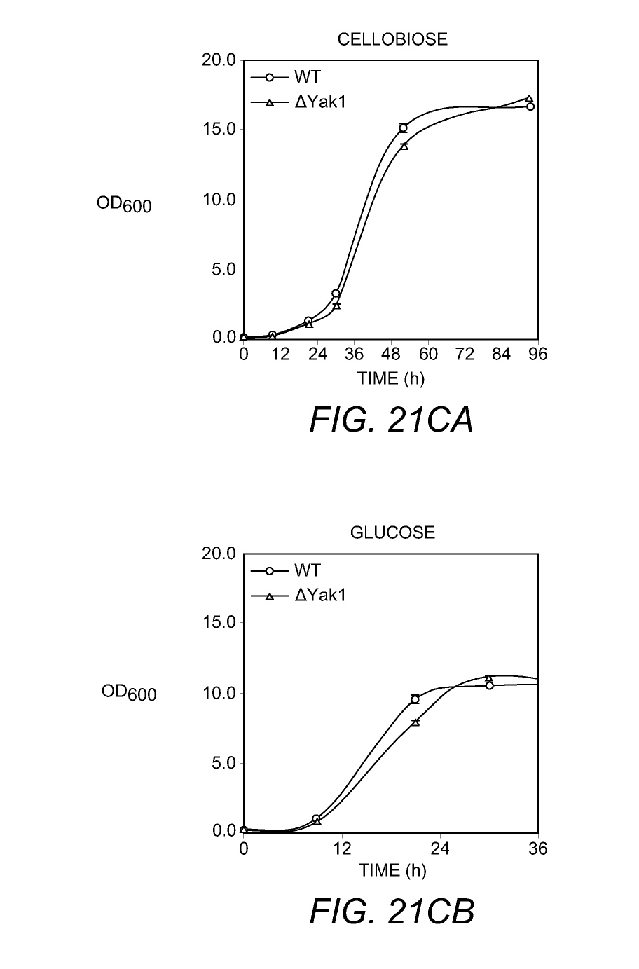

In order to determine whether yeast cells sense extracellular cellobiose through common glucose sensors, single deletion strains of Snf3, Rgt2 and Gpr1 were transformed with the cellobiose utilization pathway and their growth on cellobiose and glucose was compared (FIG. 21). It has been reported that deletion of Snf3 prevents rapid adaptation to low glucose concentration [12]. In this study, carbon source transfer experiment showed that Snf3 deletion slightly decreased growth on glucose, but had no effect on growth on cellobiose (FIG. 21). In terms of Rgt2 deletion, no significant differences were found from the wild type strain (FIG. 21). These results suggest that single deletion of Snf3 or Rgt2 has no effect on cellobiose sensing in yeast, but does not mean that cellobiose bypasses Snf3 and Rgt2. On the other hand, the Gpr1 deletion significantly decreased growth on both cellobiose and glucose (FIG. 21). This result suggests that Gpr1 can recognize cellobiose, and that the Gpr1/Gpa2 pathway may be activated by cellobiose.

Response of Intracellular Glucose Signaling Pathways to Cellobiose

Ras2 and Gpa2.

Protein kinase A (PKA) plays critical roles in growth, in response of cells to glucose, and in coupling cell cycle progression to mass accumulation. This pathway mainly induces genes that are involved in ribosomal protein synthesis, in ribosome biogenesis, in glycolysis and repression of genes that are involved in stress response, in gluconeogenesis, and in metabolism of storage carbohydrates. Induction of an activated allele of RAS2 (RAS2^{G19V}) in yeast cells growing in glycerol medium leads to the identical qualitative and quantitative changes in expression of 90% of all the genes whose expressions are altered by addition of glucose to wild-type cells [13]. Ras2^{Q77K} and RAS2^{D112Y}, two activity reduced alleles, were recently found in evolutionary stains which showed increased specific growth rate on galactose [14]. Gpr1 and Gpa2 define a nutrient-sensing pathway that works in parallel with Ras2 to activate PKA. These observations provide strong evidence that Ras2 plays a major role in mediating glucose-induced gene expression changes while Gpa2 plays a more auxiliary role in the glucose response, and both do so solely through modulation of PKA. As reported, extracellular glucose detection can be fulfilled via the constitutively active Gpa2^{G19V} allele in the absence of Gpr1 [15]. Moreover, microarray data showed that induction of GPA2^{Q300L} resulted in expression changes in the same sets of genes as did induction of an activated allele of Ras2 [16].

Sch9.

Sch9 acts in parallel to the Ras/PKA pathway but seems to serve as a minor conduit for glucose-mediated changes in transcription. Sch9 overexpression suppresses deficiencies in the PKA pathway and its inactivation results in diminished growth and reduced expression of genes in ribosomal biogenesis. Sch9 appears to serve as a major conduit by which TORC1 influences growth and mass accumulation. Sch9^{2D3E} (T723D, S726D, T737E, S758E, and S765E) is a TOR-independent SCH9 allele [17]. As such, Sch9 impinges on many of the same downstream targets as does PKA, which may account for the ability of excess Sch9 to compensate for loss of PKA activity. Both kinases act through separate signaling cascades. In addition, genome-wide expression analysis under conditions and with strains in which either PKA and/or Sch9 signaling was specifically affected, demonstrated that both kinases synergistically or oppositely regulate given gene targets [18].

Yak1.

Yak1 is a protein kinase that works in parallel to the Ras/PKA pathway in response to glucose but with the opposite effect: PKA suppresses the stress response and stimulates growth whereas Yak1 stimulates the stress response and inhibits growth [4].

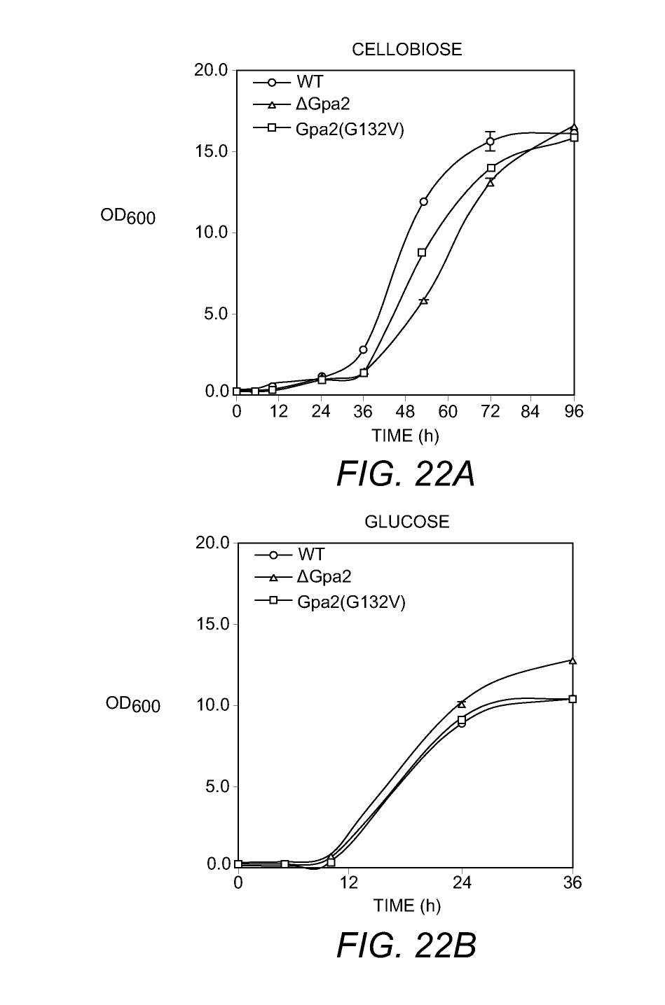

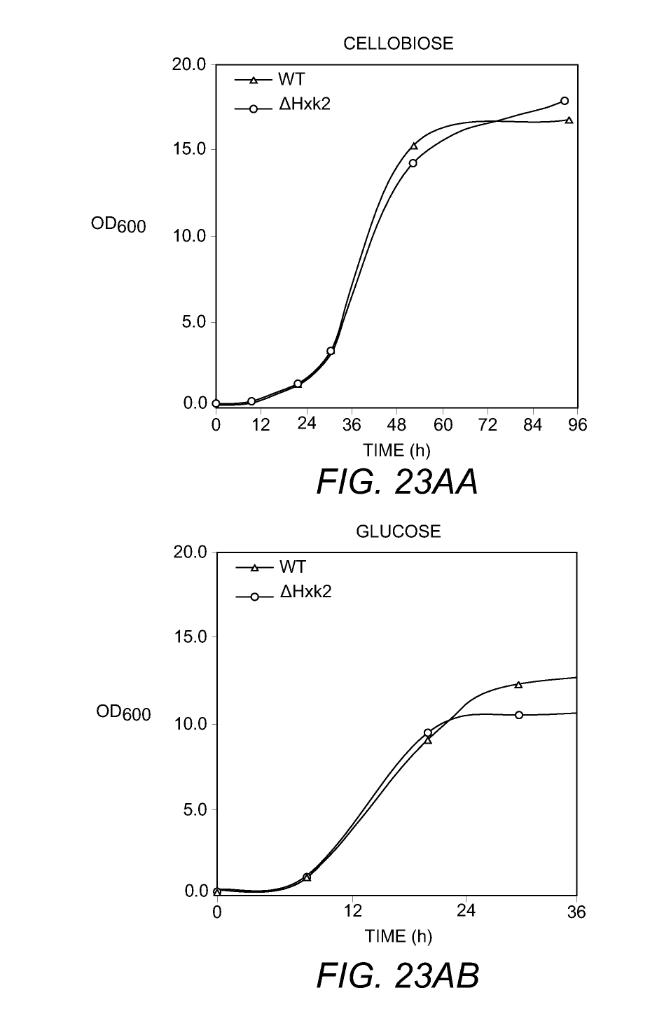

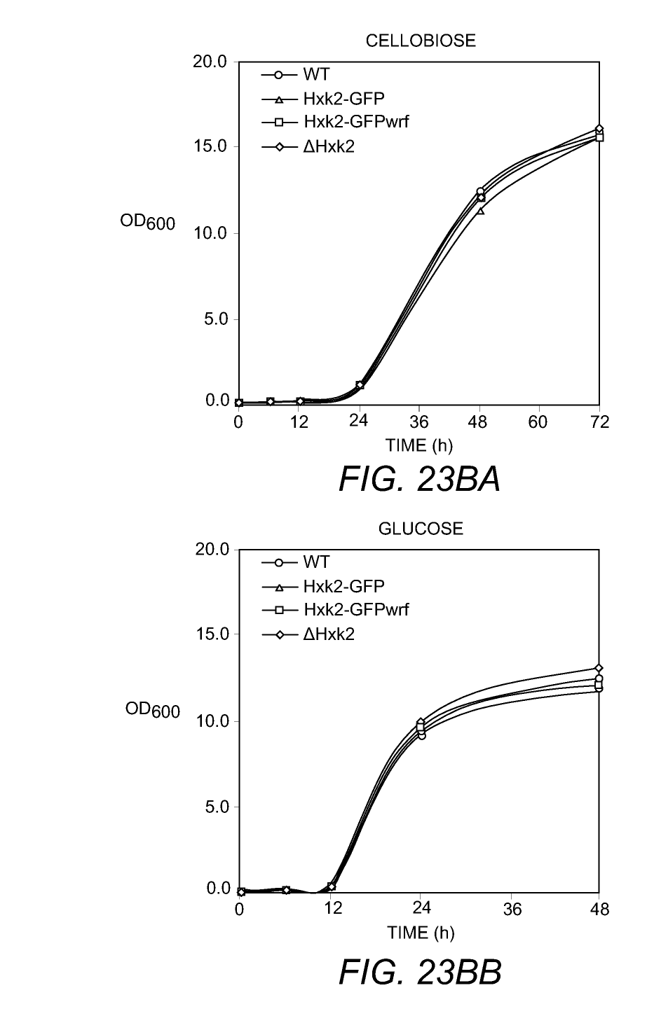

Based on carbon source transfer experiments utilizing Ras2, Gpa2, Sch9, and Yak1 single deletion strains containing the cellobiose utilization pathway, both the Ras2 and Sch9 single deletion strains significantly prolonged the lag phase on cellobiose in contrast to those on glucose (FIGS. 22A and 22B). On the other hand, the Gpa2 deletion strain had a stronger effect on cellobiose-mediated growth than glucose-mediated growth (FIG. 22A). These results suggest that cells grown on glucose have robust signaling network. The Yak1 deletion strain had a slight effect on both cellobiose-mediated growth and glucose-mediated growth, which confirmed that Yak1 plays a minor role in the signaling pathways (FIG. 22C). Interestingly, a constitutively active allele of Gpa2 (Gpa2^{G19V}) seemed to have an intermediate effect on cellobiose-mediated growth, but was similar to the wild type allele when cells were grown on glucose (FIG. 23). Cellobiose activates the intracellular glucose repression pathway

Hxk2.

Hxk2 has a double subcellular localization: it functions as a glycolytic enzyme in the cytoplasm and as a regulator of gene transcription of several Mig1-regulated genes in the nucleus. Functional studies suggest that the main regulatory role of Hxk2 is produced by interaction with the transcriptional repressor Mig1 and the Snf1 protein kinase to generate a repressor complex in the nucleus [19, 20]. The Hxk2wrf mutant allele lacking amino acids from Lys₆ to Met₁₅ was incapable of glucose repression signaling but maintained hexose-phosphorylating activity [6].

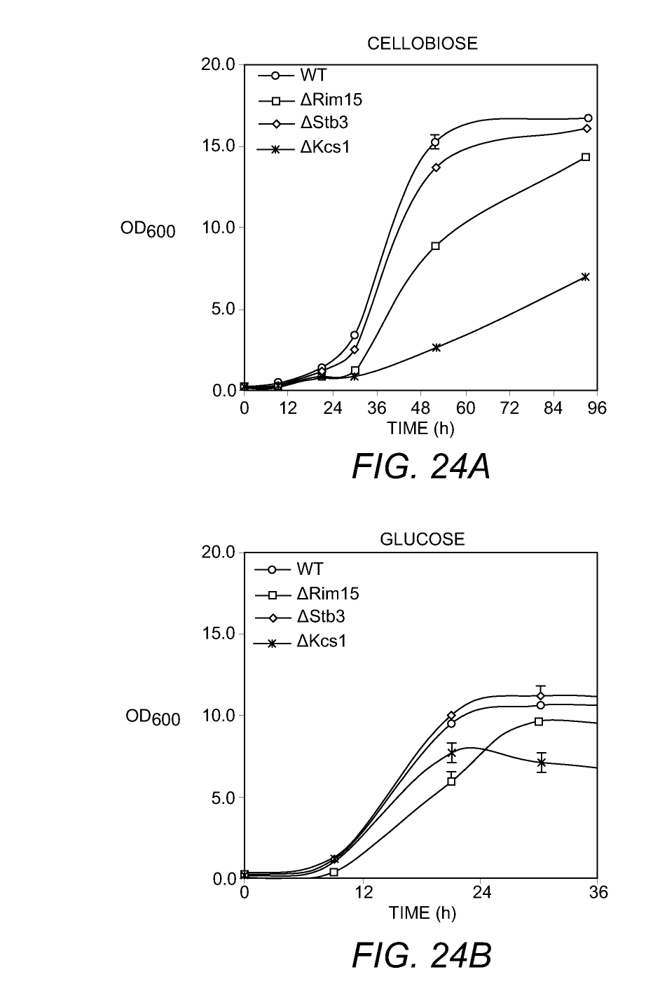

Carbon source transfer experiments indicated that the Hxk2 deletion and the Hxk2wrf mutant that lacks the Hxk2 regulatory function did not appear to affect growth on cellobiose or glucose (FIGS. 24A and 24B). These results suggest that cellobiose may activate Hxk2-mediated glucose repression as strong as glucose.

Other Key Genes Related to Cell Growth

Rim15.

Rim15 is a glucose-repressible protein kinase involved in signal transduction during cell proliferation in response to nutrients, specifically the establishment of stationary phase.

Stb3.

Stb3 is ribosomal RNA processing element (RRPE)-binding protein involved in the glucose-induced transition from quiescence to growth. Stb3 overexpression produces a slow growth phenotype [21].

Kcs1.

Kcs1 deletion leads to high concentration of ATP, and enhanced glycolytic flux and fermentation [22].

Tps1.

Glycolytic activity and gene expression are controlled by metabolic intermediates, including trehalose-6-P. Tps1 is involved in synthesis of trehalose-6-P, which inhibits Hxk2 activity [23].

Growth of the Rim15 deletion strain on both cellobiose and glucose showed decreased growth (FIG. 25). The Stb3 deletion strain grown on cellobiose and glucose seemed to show opposite results in that growth on cellobiose decreased while growth on glucose increased (FIG. 25). The Kcs1 deletion strain showed decreased growth on cellobiose (FIG. 25). The result with Kcs1 was surprising, as it was expected that deleting Kcs1 would benefit cellobiose utilization given that is has been reported that Kcs1 deletion leads to a high concentration of ATP.

### REFERENCES

1. Galazka J M, Cate J H (2011) A new diet for yeast to improve biofuel production. Bioeng Bugs 2.
2. Salusjarvi L, Kankainen M, Soliymani R, Pitkanen J P, Penttila M, et al. (2008) Regulation of xylose metabolism in recombinant Saccharomyces cerevisiae. Microbial Cell Factories 7.
3. Souto-Maior A M, Runquist D, Hahn-Hagerdal B (2009) Crabtree-negative characteristics of recombinant xylose-utilizing Saccharomyces cerevisiae. J Biotechnol 143: 119-123.
4. Zaman S, Lippman S I, Zhao X, Broach J R (2008) How Saccharomyces Responds to Nutrients. Annual Review of Genetics 42: 27-81.
5. Galazka J M, Tian C, Beeson W T, Martinez B, Glass N L, et al. (2010) Cellodextrin transport in yeast for improved biofuel production. Science 330: 84-86.
6. Pelaez R, Herrero P, Moreno F (2010) Functional domains of yeast hexokinase 2. Biochem J 432: 181-190.
7. Guthrie C, Fink G R. Amsterdam; Boston; London: Academic Press; 2002. Guide to yeast genetics and molecular and cell biology.
8. Lundblad V, Hartzog G, Moqtaderi Z (2001) Manipulation of cloned yeast DNA. Curr Protoc Mol Biol Chapter 13: Unit13 10.
9. Ozcan S, Johnston M (1999) Function and regulation of yeast hexose transporters. Microbiol. Mol Biol Rev 63: 554-569.
10. Ozcan S, Dover J, Johnston M (1998) Glucose sensing and signaling by two glucose receptors in the yeast Saccharomyces cerevisiae. EMBO J. 17: 2566-2573.
11. Gancedo J M (2008) The early steps of glucose signalling in yeast. FEMS Microbiol Rev 32: 673-704.
12. Ramakrishnan V, Theodoris G, Bisson L F (2007) Loss of IRA2 suppresses the growth defect on low glucose caused by the snf3 mutation in Saccharomyces cerevisiae. FEMS Yeast Res 7: 67-77.
13. Wang Y, Pierce M, Schneper L, Guldal C G, Zhang X, et al. (2004) Ras and Gpa2 mediate one branch of a redundant glucose signaling pathway in yeast. PLoS Biol 2: E128.
14. Hong K K, Vongsangnak W, Vemuri G N, Nielsen J (2011) Unravelling evolutionary strategies of yeast for improving galactose utilization through integrated systems level analysis. Proc Natl Acad Sci USA 108: 12179-12184.
15. Rolland F, De Winde J H, Lemaire K, Boles E, Thevelein J M, et al. (2000) Glucose-induced cAMP signalling in yeast requires both a G-protein coupled receptor system for extracellular glucose detection and a separable hexose kinase-dependent sensing process. Mol Microbiol 38: 348-358.
16. Peeters T, Louwet W, Gelade R, Nauwelaers D, Thevelein J M, et al. (2006) Kelch-repeat proteins interacting with the Galpha protein Gpa2 bypass adenylate cyclase for direct regulation of protein kinase A in yeast. Proc Natl Acad Sci USA 103: 13034-13039.
17. Urban J, Soulard A, Huber A, Lippman S, Mukhopadhyay D, et al. (2007) Sch9 is a major target of TORC1 in Saccharomyces cerevisiae. Molecular Cell 26: 663-674.
18. Roosen J, Engelen K, Marchal K, Mathys J, Griffioen G, et al. (2005) PKA and Sch9 control a molecular switch important for the proper adaptation to nutrient availability. Mol Microbiol 55: 862-880.
19. Ahuatzi D, Herrero P, de la Cera T, Moreno F (2004) The glucose-regulated nuclear localization of hexokinase 2 in Saccharomyces cerevisiae is Mig1-dependent. J Biol Chem 279: 14440-14446.
20. Moreno F, Ahuatzi D, Riera A, Palomino C A, Herrero P (2005) Glucose sensing through the Hxk2-dependent signalling pathway. Biochem Soc Trans 33: 265-268.
21. Liko D, Conway M K, Grunwald D S, Heideman W (2010) Stb3 plays a role in the glucose-induced transition from quiescence to growth in Saccharomyces cerevisiae. Genetics 185: 797-810.
22. Szijgyarto Z, Garedew A, Azevedo C, Saiardi A (2011) Influence of inositol pyrophosphates on cellular energy dynamics. Science 334: 802-805.
23. Rolland F, Baena-Gonzalez E, Sheen J (2006) Sugar sensing and signaling in plants: conserved and novel mechanisms. Annu Rev Plant Biol 57: 675-709.

1. A method for degrading cellodextrin, comprising:
a) providing a host cell comprising a recombinant cellodextrin transporter and a recombinant polypeptide comprising Y-x(2)-G-x-[KR]-E-N-[AG]-[AG]-[IV]-F-x(2)-[ANST]-[NST]-x(2)-[AIV]-x(2)-[AGT]-x(4)-[AG]-x(4)-[ADNS] (SEQ ID NO: 233), Y-Q-[CN]-M-[IV]-T-F-[CN]-[FILMV]-[AS]-RS-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 14), or G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 15), wherein the recombinant polypeptide has cellodextrin phosphorylase activity; and
b) culturing the host cell in a medium comprising cellodextrin or a source of cellodextrin, whereby cellodextrin is transported into the cell and degraded by said recombinant polypeptide.

2. (canceled)

3. (canceled)

4. The method of claim 1, wherein the recombinant polypeptide comprises an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell.

5. The method of claim 1, wherein the recombinant polypeptide has cellobiose phosphorylase activity.

6. The method of claim 5, wherein the recombinant polypeptide comprises an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from the group consisting of SEQ ID NO: 11 (CgCBP), SEQ ID NO: 12 (SdCBP), and SEQ ID NO: 13 (CtCBP).

7. (canceled)

8. (canceled)

9. The method of claim 1, wherein the recombinant polypeptide comprises an amino acid substitution at one or more positions corresponding to positions of the amino acid sequence of SEQ ID NO: 12 (SdCBP), wherein the one or more amino acid substitutions are selected from the group consisting of an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof.

10. The method of claim 1, wherein the host cell further comprises a recombinant phosphoglucomutase.

11. The method of claim 10, wherein the recombinant phosphoglucomutase comprises a conserved motif having the amino acid sequence of [GSA]-[LIVMF]-x-[LIVM]-[ST]-[PGA]-S-H-[NIC]-P (SEQ ID NO: 19).

12. The method of claim 1, wherein the host cell further comprises a recombinant hexokinase.

13. The method of claim 12, wherein the recombinant hexokinase comprises a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20).

14. The method of claim 13, wherein the recombinant hexokinase is HXK1.

15.-150. (canceled)

151. A host cell comprising a recombinant cellodextrin transporter, and a recombinant polypeptide comprising G-x(2)-[FY]-x-N-[AGS]-x-[AS]-W-[APS]-V-[IL]-[AS]-x(2)-A-x(2)-[DE]-x-[AI]-x(3)-[LMV]-[DEN]-[ASV]-[ILV]-x(3)-L-x-T-x(2)-G-[ILV]-x(2)-[SV]-x-P-[AG] (SEQ ID NO: 14) or Y-Q-[CN]-M-[IV]-T-F-[CN]-[FILMV]-[AS]-R-[ST]-[AS]-S-[FY]-[FY]-E-[STV]-G-x-[GS]-R-G-[IM]-G-F-R-D-S-[ACNS]-Q-D-[ILV]-[ILMV]-G-x-V-H-x-[IV]-P-[ADEST]-x-[AV]-[KR]-[AEQ]-x-[IL]-[FIL]-D (SEQ ID NO: 15), wherein the recombinant polypeptide has cellodextrin phosphorylase activity.

152. The host cell of claim 151, wherein the recombinant polypeptide comprises an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to the amino acid sequence of CDP_Clent, CDP_Ctherm, or CDP_Acell.

153. The host cell of claim 151, wherein the recombinant polypeptide has cellobiose phosphorylase activity.

154. The host cell of claim 153, wherein the recombinant polypeptide comprises an amino acid sequence that has at least 29%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, or at least 100% amino acid identity to an amino acid sequence selected from the group consisting of SEQ ID NO: 11 (CgCBP), SEQ ID NO: 12 (SdCBP), and SEQ ID NO: 13 (CtCBP).

155. (canceled)

156. (canceled)

157. The host cell of claim 151, wherein the recombinant polypeptide comprises an amino acid substitution at one or more positions corresponding to positions of the amino acid sequence of SEQ ID NO: 12 (SdCBP), wherein the one or more amino acid substitutions are selected from the group consisting of an isoleucine (I) to glutamine (Q) substitution at a position corresponding to amino acid 409 of SEQ ID NO: 12; an isoleucine (I) to methionine (M) substitution at a position corresponding to amino acid 409 of SEQ ID NO: 12; an asparagine (N) to aspartate (D) substitution at a position corresponding to amino acid 482 of SEQ ID NO: 12; an asparagine (N) to threonine (T) substitution at a position corresponding to amino acid 482 of SEQ ID NO: 12; a cysteine (C) to serine (S) substitution at a position corresponding to amino acid 484 of SEQ ID NO: 12; a cysteine (C) to alanine (A) substitution at a position corresponding to amino acid 484 of SEQ ID NO: 12; a phenylalanie (F) to tryptophan (W) substitution at a position corresponding to amino acid 651 of SEQ ID NO: 12; a histidine (H) to asparagine (N) substitution at a position corresponding to amino acid 653 of SEQ ID NO: 12; a histidine (H) to alanine (A) substitution at a position corresponding to amino acid 653 of SEQ ID NO: 12; and combinations thereof.

158. The host cell of claim 151, wherein the host cell further comprises a recombinant phosphoglucomutase.

159. The host cell of claim 158, wherein the recombinant phosphoglucomutase comprises a conserved motif having the amino acid sequence of [GSA]-[LIVMF]-x-[LIVM]-[ST]-[PGA]-S-H-[NIC]-P (SEQ ID NO: 19).

160. The host cell of claim 151, wherein the host cell further comprises a recombinant hexokinase.

161. The host cell of claim 160, wherein the recombinant hexokinase comprises a conserved motif having the amino acid sequence of [LIVM]-G-F-[TN]-F-S-[FY]-P-x(5)-[LIVM]-[DNST]-x(3)-[LIVM]-x(2)-W-T-K-x-[LF] (SEQ ID NO: 20).

162. The host cell of claim 161, wherein the recombinant hexokinase is HXK1.

163-279. (canceled)

280. The method of claim 1, wherein hydrocarbons or hydrocarbon derivatives are produced from the cellodextrin.
